(19) United States
(12) Patent Application Publication
(10) Pub. No.: US 2010/0168105 A1
(43) Pub. Date: Jul. 1, 2010

(54) Spirocyclopropyl Piperidine Derivatives

(75) Inventors: Peter Bernstein, Wilmington, DE (US); Dean Brown, Wilmington, DE (US); Joseph Cacciola, Wilmington, DE (US); Phil Edwards, Wilmington, DE (US); James Folmer, Wilmington, DE (US); Thierry Groblewski, Montreal (CA); Mark Sylvester, Wilmington, DE (US); Steven Wesolowski, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/601,308

(22) PCT Filed: May 23, 2008
(86) PCT No.: PCT/SE08/50606

§ 371 Date: Nov. 23, 2009

Related U.S. Application Data
(60) Provisional application No. 60/940,350, filed on May 25, 2007.

(51) Int. Cl. A61K 31/5377; C07D 401/14; A61K 31/496; A61P 25/18; A61P 25/28; A61P 3/04

(52) U.S. Cl. 514/235.8; 544/230; 544/70; 514/253.11

(57) ABSTRACT

Disclosed herein is at least one piperidine derivative, at least one pharmaceutical composition comprising at least one piperidine derivative disclosed herein for treating at least one histamine H3 receptor associated condition therewith

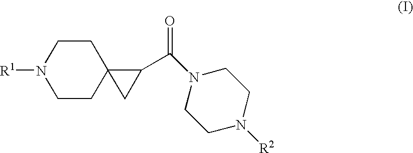

Disclosed herein is at least one piperidine derivative, at least one pharmaceutical composition comprising at least one piperidine derivative disclosed herein, and at least one method of using at least one piperidine derivative disclosed herein for treating at least one histamine H3 receptor associated condition therewith.

The histamine H3 receptor is of current interest in developing new medicaments. The H3 receptor is a presynaptic autoreceptor located both in the central and peripheral nervous systems, the skin, and in organs, such as, for example, the lung, the intestine, probably the spleen, and the gastrointestinal tract. Recent evidence suggests the H3 receptor has intrinsic, constitutive activity in vitro as well as in vivo (i.e., it is active in the absence of an agonist). Compounds acting as inverse agonists can inhibit this activity. The histamine H3 receptor has been shown to regulate the release of histamine and also of other neurotransmitters, such as, for example, serotonin and acetylcholine. Some histamine H3 ligands, such as, for example, a histamine H3 receptor antagonist or inverse agonist may increase the release of neurotransmitters in the brain, whereas other histamine H3 ligands, such as, for example, histamine H3 receptor agonists may inhibit the biosynthesis of histamine, as well as, inhibit the release of neurotransmitters. This suggests that histamine H3 receptor agonists, inverse agonists, and antagonists could mediate neuronal activity. As a result, efforts have been undertaken to develop new therapeutics that target the histamine H3 receptor.

Described herein are compounds of formula I, enantiomers thereof, pharmaceutically acceptable salts thereof, or mixtures thereof:

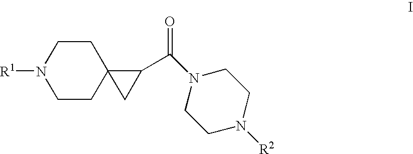

wherein:

R¹ is aryl, heteroaryl, arylalkyl, heteroarylalkyl, cycloalkyl, heterocycloalkylalkyl, alkyl, alkoxyalkyl, hydroxyalkyl, aminoalkyl, aminocarbonylalkyl, heterocycle, arylalkenyl, or heterocycloalkyl; wherein R¹ is optionally substituted with at least one substituent selected independently from alkyl, cyano, sulfinyl, haloalkyl, amide, alkoxy, halogen, arylalkoxy, alkylcarbonyl, carboxy (—C(═O)OH), hydroxy (—OH), amino, alkoxycarbonyl, and alkylsulfonyl; and

R² is aryl, heteroaryl, cycloalkyl, alkyl, heterocycloalkyl, or cycloalkylalkyl; wherein R² is optionally substituted with at least one substituent selected independently from C₁-C₆alkyl, alkoxy, and cycloalkyl;

with the provisos that:

1) when R² is a cyclohexyl, R¹ is not a substituted or unsubstituted phenyl;

2) when R² is aryl, R² is not unsubstituted phenyl; and

when R¹ is tetrahydropyran-4-yl and R² is a substituted phenyl, the substituted phenyl is not ortho- or meta-substituted with methyl or meta-substituted with methoxy.

Further described herein are compounds according to formula I for use as medicaments.

Even further described herein is the use of compounds according to formula I in the manufacture of a medicament for the therapy of at least one disorder selected from cognitive deficient in schizophrenia, narcolepsy, pain, obesity, and Alzheimer's disease.

Yet even further described herein is a pharmaceutical composition comprising at least one compound according to formula I and a pharmaceutically acceptable carrier and/or diluent.

Still even further described herein are compounds according to formula I for use in treating at least one disorder selected from cognitive deficient in schizophrenia, narcolepsy, obesity, pain, and Alzheimer's disease.

Still yet even further described herein is a method for treating at least one disorder selected from cognitive deficient in schizophrenia, narcolepsy, pain, obesity, and Alzheimer's disease in a warm-blooded animal, comprising administering to said animal in need of such treatment a therapeutically effective amount of at least one compound according to formula I.

Still yet even further described herein is a method for treating a disorder in which modulating the histamine H3 receptor is beneficial comprising administering to a warm-blooded animal in need of such treatment a therapeutically effective amount of at least one compound according to formula I.

The features and advantages of the invention may be more readily understood by those of ordinary skill in the art upon reading the following detailed description. It is to be appreciated that certain features of the invention that are, for clarity reasons, described above and below in the context of separate embodiments, may also be combined to form a single embodiment. Conversely, various features of the invention that are, for brevity reasons, described in the context of a single embodiment, may also be combined so as to form sub-combinations thereof.

Unless specifically stated otherwise herein, references made in the singular may also include the plural. For example, “a” and “an” may refer to either one, or one or more.

Embodiments identified herein as exemplary are intended to be illustrative and not limiting.

Unless otherwise indicated, any heteroatom with unsatisfied valences is assumed to have hydrogen atoms sufficient to satisfy the valences.

The definitions set forth herein take precedence over definitions set forth in any patent, patent application, and/or patent application publication incorporated herein by reference.

Definitions of terms used in describing the invention are set forth hereinbelow. Unless otherwise indicated, the initial definition provided for a group or term applies each time such group or term is used individually or as part of another group.

Throughout the specification, groups and substituents thereof may be chosen by one skilled in the field to provide stable moieties and compounds.

Unless specified otherwise herein, the nomenclature used herein generally follows the examples and rules stated in Nomenclature of Organic Chemistry, Sections A, B, C, D, E, F, and H, Pergamon Press, Oxford, 1979.

The term “Cₘ-Cₙ” or “Cₘ-Cₙ group” used alone or as a prefix, refers to any group having m to n carbon atoms. For example, the term “C₁-C₄alkyl” refers to an alkyl group containing from 1 to 4 carbon atoms.

The terms “alkyl” and “alk” refer to a straight or branched chain alkane (hydrocarbon) radical containing from 1 to 12 carbon atoms. Exemplary “alkyl” and “alk” groups include, but are not limited to, for example, methyl; ethyl; propyl; isopropyl; 1-methylpropyl; n-butyl, t-butyl; isobutyl; pentyl; hexyl; isohexyl; heptyl; 4,4-dimethylpentyl; diethylpentyl; octyl; 2,2,4-trimethylpentyl; nonyl; decyl; undecyl; and dodecyl.

The term “hydrocarbon” refers to a chemical structure comprising only carbon and hydrogen atoms.

The term “hydrocarbon radical” refers to a hydrocarbon that has had at least one hydrogen removed therefrom.

The term “lower alkyl” refers to an alkyl group containing from 1 to 4 carbon atoms. It is of import to note that the term “lower alkyl” is encompassed within the definition of “alkyl”. The usage of the term “lower alkyl”, however, is not intended to limit the definition of the term “alkyl” either explicitly or implicitly to a straight- or branched-chain saturated hydrocarbon radical containing from 5 to 12 carbon atoms. Exemplary lower alkyl groups include, but are not limited to, for example, methyl; ethyl; propyl; isopropyl; n-butyl; t-butyl; and isobutyl.

The term “aryl” refers to monocyclic or bicyclic aromatic hydrocarbon rings having from 6 to 12 carbon atoms in the ring portion. Exemplary aryl groups include but are not limited to, for example, phenyl; naphthalenyl; biphenyl; and diphenyl. When two aromatic rings are present, the aromatic rings of the aryl group may either be joined at a single point (e.g., biphenyl), or be fused (e.g., naphthalenyl).

The term “heteroaryl” refers to aromatic cyclic groups, such as, for example, 5- to 6-membered monocyclic, 7- to 11-membered bicyclic, or 10- to 16-membered tricyclic ring systems having at least one heteroatom in at least one carbon atom-containing ring. The carbon atom-containing ring may contain 1, 2, 3, or 4 heteroatom(s) selected from nitrogen, oxygen, and sulfur. The heteroaryl group may be attached to another moiety at any available point of attachment.

Exemplary monocyclic heteroaryl groups include, but are not limited to, for example, pyrazolyl, imidazolyl, triazolyl, oxazolyl, furyl, thiazolyl, isoxazolyl, thiazolyl, pyridyl, pyridazinyl, pyrimidinyl, pyrazinyl, and triazinyl. Unless reference is made to a specific point of attachment, e.g., as in pyrid-2-yl, pyridazin-3-yl, it is intended that such heteroaryl groups can be bonded to another moiety at any available point of attachment.

Exemplary bicyclic heteroaryl groups include, but are not limited to, for example, benzothiazolyl, benzoxazolyl, benzoxadiazolyl, benzothienyl, quinolinyl, chromenyl, indolyl, indazolyl, isoquinolinyl, benzimidazolyl, benzopyranyl, benzofuryl, benzofurazanyl, benzopyranyl, cinnolinyl, quinoxalinyl, pyrrolopyridyl, furopyridinyl, and triazinylazepinyl.

The term “cycloalkyl” refers to a fully saturated and partially unsaturated cyclic hydrocarbon group containing from 1 to 3 rings and 3 to 8 carbons per ring. Exemplary cycloalkyls include, but are not limited to, for example, cyclopropyl, cyclobutyl, cyclopentyl, cyclohexyl, cycloheptyl, cyclooctyl, cyclobutenyl, cyclopentenyl, and cyclohexenyl. A cycloalkyl ring may have a carbon ring atom replaced with a carbonyl group (C═O). Cycloalkyls include rings having a second or third ring fused thereto that is a heterocyclo, heteroaryl, or aryl, provided that in such cases the point of attachment is to the cycloalkyl portion of the ring system. The term “cycloalkyl” also includes rings having a second or third ring attached to the ring or ring system in a spiro fashion.

Exemplary cycloalkyls include, but are not limited to, for example,

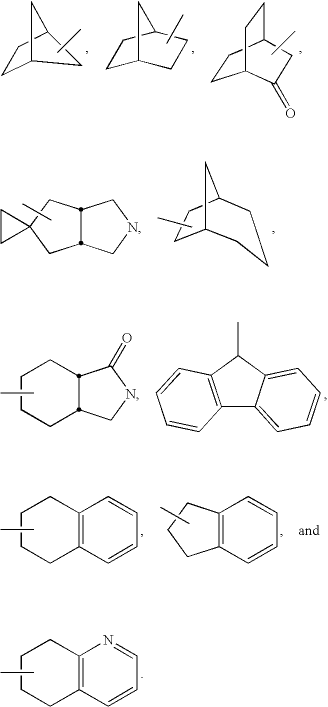

The terms “heterocycle” or “heterocyclo” refer to an optionally substituted, fully saturated or unsaturated, aromatic or nonaromatic cyclic group, which is, for example, a 4 to 7 membered monocyclic, 7 to 11 membered bicyclic, or 10 to 15 membered tricyclic ring system that has at least one heteroatom in at least one carbon atom-containing ring. Each ring of the heterocycle or heterocyclo containing a heteroatom may have 1, 2 or 3 heteroatoms selected from N, O, and S, where the N and S heteroatoms may optionally be oxidized and the N heteroatom may optionally be quaternized. The heterocycle or heterocyclo may be attached via any heteroatom or carbon atom.

Exemplary monocyclic heterocycle(s) or heterocyclo(s) include, but are not limited to, for example, pyrrolidinyl, pyrrolyl, indolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, homopiperazinyl, 2-oxohomopiperazinyl, 2-oxopyrrolidinyl, 2-oxazepinyl, azepinyl, 4-piperidonyl, pyridyl, N-oxo-pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, dioxanyl, isothiazolidinyl, thietanyl, thiiranyl, triazinyl, and triazolyl.

Exemplary bicyclic heterocycle(s) or heterocyclo(s) include, but are not limited to, for example, benzothiazolyl, benzoxazolyl, benzothienyl, benzodioxole, quinuclidinyl, quinolinyl, quinolinyl-N-oxide, tetrahydroisoquinolinyl, isoquinolinyl, benzimidazolyl, benzopyranyl, indolizinyl, benzofuryl, chromonyl, coumarinyl, cinnolinyl, quinoxalinyl, indazolyl, pyrrolopyridyl, furopyridinyl, dihydroisoindolyl, dihydroquinazolinyl, benzisothiazolyl, benzisoxazolyl, benzodiazinyl, benzofurazanyl, benzothiopyranyl, benzotriazolyl, benzpyrazolyl, dihydrobenzofuryl, dihydrobenzothienyl, dihydrobenzothiopyranyl, dihydrobenzothiopyranyl sulfone, dihydrobenzopyranyl, indolinyl, indazolyl, isochromanyl, isoindolinyl, naphthyridinyl, phthalazinyl, piperonyl, purinyl, pyridopyridyl, quinazolinyl, tetrahydroquinolinyl, thienofuryl, thienopyridyl, and thienothienyl.

The term “heterocycloalkyl” refers to a saturated or unsaturated cycloalkyl in which at least one ring carbon (and any associated hydrogen atoms) are independently replaced with at least one heteroatom selected from O and N. In another embodiment, the at least one ring carbon (and any associated hydrogen atoms) are independently replaced with at least one heteroatom selected from O, N, and S. In yet another embodiment, at least two ring carbons (and any associated hydrogen atoms) are each independently replaced with at least two heteroatoms selected from O, N, and S.

The term “alkenyl” refers to a straight or branched chain hydrocarbon radical containing from 2 to 12 carbon atoms and at least one carbon-carbon double bond. Exemplary alkenyls include, but are not limited to, for example, ethenyl and allyl.

The terms “halogen” and “halo” refer to chlorine, bromine, fluorine, and iodine.

The term “haloalkyl” refers to an alkyl bonded to a single halogen or multiple halogens. Exemplary haloalkyls containing multiple halogens include, but are not limited to, for example, —CHCl₂ and —CF₃.

The term “amide” refers to the group —C(═O)NR^{e}R^{f}, wherein R^{e} and R^{f} are independently selected from H, alkyl, alkenyl, and cycloalkyl.

The term “amine” or “amino” used alone or as a suffix or prefix, refers to radicals of the general formula —NR^{g}R^{h}, wherein R^{g} and R^{h} are independently selected from hydrogen or a hydrocarbon radical.

The term “alkoxy” used alone or as a suffix or prefix, refers to radicals of the general formula —ORⁱ, wherein Rⁱ is selected from a hydrocarbon radical. Exemplary alkoxys include, but are not limited to methoxy, ethoxy, propoxy, isopropoxy, butoxy, t-butoxy, isobutoxy, cyclopropylmethoxy, allyloxy, and propargyloxy.

The term “sulfinyl” refers to an S(═O).

The term “sulfonyl” refers to the group S(═O)₂.

The term “carbonyl” refers to the group C(═O).

The term “optionally substituted” refers to either groups, structures, or molecules that are substituted with at least one substituent at any available and substitutable position and groups, structures, or molecules that are not substituted.

The term “pharmaceutically-acceptable”, as employed herein, indicates the subject matter being identified as “pharmaceutically acceptable” is suitable and physiologically acceptable for administration to a patient/subject. For example, the term “pharmaceutically acceptable salt(s)” denotes suitable and physiologically acceptable salt(s).

The phrase “a compound of formula I, enantiomers thereof, pharmaceutically acceptable salts thereof, or mixtures thereof” refers to the free base of formula I or enantiomers thereof, pharmaceutically acceptable salts of formula I or enantiomers thereof, and/or mixtures of at least one free base of formula I or enantiomers thereof and at least one pharmaceutically acceptable salt of formula I or enantiomers thereof.

The term “therapeutically-effective amount” refers to that amount of a compound sufficient to modulate one or more of the symptoms of the condition or disease being treated.

In one aspect, the invention provides a compound of formula I, enantiomers thereof, pharmaceutically acceptable salts thereof, or mixtures thereof:

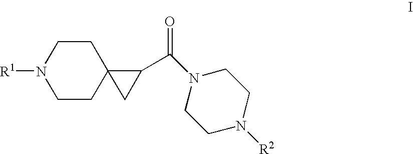

wherein:

R¹ is aryl, heteroaryl, arylalkyl, heteroarylalkyl, cycloalkyl, heterocycloalkylalkyl, alkyl, alkoxyalkyl, hydroxyalkyl, aminoalkyl, aminocarbonylalkyl, heterocycle, arylalkenyl, or heterocycloalkyl; wherein R¹ is optionally substituted with at least one substituent selected independently from alkyl, cyano, sulfinyl, haloalkyl, amide, alkoxy, halogen, arylalkoxy, alkylcarbonyl, carboxy (—C(═O)OH), hydroxy (—OH), amino, alkoxycarbonyl, and alkylsulfonyl; and

R² is aryl, heteroaryl, cycloalkyl, alkyl, heterocycloalkyl, or cycloalkylalkyl; wherein R² is optionally substituted with at least one substituent selected independently from C₁-C₆alkyl, alkoxy, and cycloalkyl;

with the provisos that:
1) when R² is a cyclohexyl, R¹ is not a substituted or unsubstituted phenyl;
2) when R² is aryl, R² is not unsubstituted phenyl; and
3) when R¹ is tetrahydropyran-4-yl and R² is a substituted phenyl, the substituted phenyl is not ortho- or meta-substituted with methyl or meta-substituted with methoxy.

In another aspect, the invention provides a compound of formula I, enantiomers thereof, pharmaceutically acceptable salts thereof, or mixtures thereof:

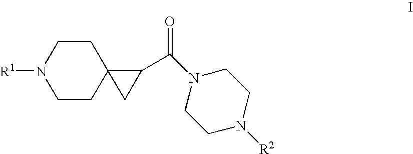

wherein:

R¹ is aryl, heteroaryl, arylalkyl, heteroarylalkyl, cycloalkyl, heterocycloalkylalkyl, alkyl, alkoxyalkyl, hydroxyalkyl, aminoalkyl, aminocarbonylalkyl, heterocycle, arylalkenyl, or heterocycloalkyl; wherein R¹ is optionally substituted with at least one substituent selected independently from alkyl, cyano, sulfinyl, haloalkyl, amide, alkoxy, halogen, arylalkoxy, alkylcarbonyl, carboxy (—C(═O)OH), hydroxy (—OH), amino, alkoxycarbonyl, and alkylsulfonyl; and

R² is aryl, heteroaryl, cycloalkyl, alkyl, heterocycloalkyl, or cycloalkylalkyl; wherein R² is optionally substituted with at least one substituent selected independently from C₁-C₆alkyl, alkoxy, and cycloalkyl;

with the provisos that:
1) when R² is a cyclohexyl, R¹ is not a substituted or unsubstituted phenyl;
2) when R² is aryl, R² is not unsubstituted phenyl;
3) when R¹ is tetrahydropyran-4-yl and R² is a substituted phenyl, the substituted phenyl is not ortho- or meta-substituted with methyl or meta-substituted with methoxy; and
4) when R² is a cyclohexyl, R¹ is not benzo[d][1,3]dioxol-5-yl or 5-(trifluoromethyl)-pyridine-2-yl.

In yet another aspect, the invention provides a compound of formula I, or a pharmaceutically acceptable salt thereof:

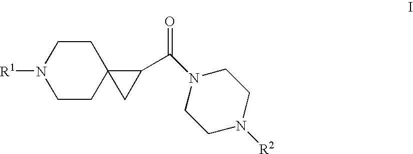

wherein:

R¹ is aryl, heteroaryl, arylalkyl, heteroarylalkyl, cycloalkyl, heterocycloalkylalkyl, alkyl, alkoxyalkyl, hydroxyalkyl, aminoalkyl, aminocarbonylalkyl, heterocycle, arylalkenyl, or heterocycloalkyl; wherein R¹ is optionally substituted with at least one substituent selected independently from alkyl, cyano, sulfinyl, haloalkyl, amide, alkoxy, halogen, alkylcarbonyl, hydroxy (—OH), amino; and

R² is a cycloalkyl, alkyl, heterocycloalkyl, or cycloalkylalkyl; wherein R² is optionally substituted with at least one cycloalkyl.

In one embodiment, R¹ is aryl, heteroaryl, arylalkyl, heteroarylalkyl, cycloalkyl, heterocycloalkylalkyl, alkyl, alkoxyalkyl, hydroxyalkyl, aminoalkyl, aminocarbonylalkyl, heterocycle, arylalkenyl, or heterocycloalkyl.

In another embodiment, R¹ is aryl, heteroaryl, arylalkyl, cycloalkyl, heterocycloalkylalkyl, alkyl, arylalkenyl, heterocycle, or heterocycloalkyl.

In still another embodiment, R¹ is aryl, heteroaryl, arylalkyl, cycloalkyl, heterocycloalkylalkyl, alkyl, arylalkenyl, or heterocycloalkyl.

In yet another embodiment, R¹ is C₆-C₁₄aryl, C₆-C₁₄heteroaryl, (C₆-C₁₄aryl)-(C₁-C₆alkyl), C₃-C₈cycloalkyl, (C₃-C₈heterocycloalkyl)-(C₁-C₆alkyl), C₁-C₆alkyl, (C₆-C₁₄aryl)-(C₁-C₆alkenyl), or C₃-C₈heterocycloalkyl.

In a further embodiment, R¹ is arylalkyl, cycloalkyl, heterocycloalkyl, or heterocycloalkylalkyl.

In another embodiment, R¹ is heterocycloalkyl, alkyl, arylalkenyl, or arylalkyl.

In still another embodiment, R¹ is heterocycloalkyl, C₁-C₆alkyl, arylalkenyl, or arylalkyl.

In yet a further embodiment, R¹ is aryl.

In an even further embodiment, R¹ is heteroaryl.

In still an even further embodiment, R¹ is arylalkyl.

In yet an even further embodiment, R¹ is cycloalkyl.

In still another embodiment, R¹ is heterocycloalkyl. In yet another embodiment, R¹ is alkyl.

In yet even another embodiment, R¹ is C₁-C₆alkyl.

In still yet another embodiment, R¹ is heterocycloalkylalkyl.

In a further embodiment, R¹ is arylalkenyl.

In yet a further embodiment, R¹ is a heterocycle.

In a still further embodiment, R¹ is piperidinyl, tetrahydropyranyl, phenylethyl, butyl, phenylallyl, cyclohexyl, tetrahydro-2H-thiopyranyl, morpholinylethyl, phenylmethyl, tetrahydrofuranyl, pyridinyl, methyl, cyclobutyl, phenyl, ethyl, or benzo[d][1,3]dioxolyl.

In yet still a further embodiment, R¹ is piperidin-yl-4-yl, tetrahydropyran-4-yl, tetrahydropyran-3-yl, phenylethyl, butyl, phenylallyl, cyclohexyl, tetrahydro-2H-thiopyran-3-yl, N-morpholinylethyl, phenylmethyl, tetrahydrofuran-3-yl, pyridin-2-yl, pyridin-4-yl, pyridin-2-yl-5-yl, pyridin-2-yl-6-yl, methylyl, cyclobutyl, phenyl-4-yl, ethylyl, or benzo[d][1,3]dioxol-5-yl.

In yet still a further embodiment, R¹ is tetrahydropyran-4-yl, phenethyl, cinnamyl, 2-ethylbutyl, or 1-methyl-4-piperidyl.

In a further embodiment, R¹ is tetrahydropyran-4-yl.

In another embodiment, R¹ is phenethyl.

In still another embodiment, R¹ is cinnamyl.

In still yet another embodiment, R¹ is 2-ethylbutyl.

In a still further embodiment, R¹ is 1-methyl-4-piperidyl.

In yet a further embodiment, R¹ is substituted with at least one substituent selected independently from alkyl, cyano, sulfinyl, haloalkyl, amide, alkoxy, halogen, alkylcarbonyl, hydroxy (—OH), and amino.

In yet another embodiment, R¹ is substituted with at least one substituent selected independently from alkyl, haloalkyl, amide, alkoxy, halogen, arylalkoxy, alkoxycarbonyl, and alkylsulfonyl.

In still yet another embodiment, R¹ is substituted with at least one substituent selected independently from C₁-C₆alkyl, haloC₁-C₆alkyl, amide, C₁-C₆alkoxy, halogen, (C₆-C₁₄aryl)-(C₁-C₆alkoxy), C₁-C₆alkoxycarbonyl, and C₁-C₆alkylsulfonyl.

In a further embodiment, R¹ is substituted with at least one substituent selected independently from ethyl, methyl, isopropyl, isopropoxycarbonyl, N-methylformamide, methylsulfonyl, methoxy, trifluoromethyl, chloro, methoxycarbonyl, and phenylmethoxy.

In a still further embodiment, R¹ is substituted with at least one alkyl.

In yet another embodiment, R¹ is substituted with at least one lower alkyl.

In yet still another embodiment, R¹ is substituted with at least one methyl.

In still yet another embodiment, R² is aryl.

In another embodiment, R² is heteroaryl.

In another embodiment, R² is cycloalkyl.

In a further embodiment, R² is alkyl.

In an even further embodiment, R² is heterocycloalkyl.

In yet an even further embodiment, R² is cycloalkylalkyl.

R² is C₆-C₁₄aryl, C₆-C₁₄heteroaryl, C₃-C₈cycloalkyl, C₁-C₆alkyl, C₃-C₈heterocycloalkyl, or (C₃-C₈cycloalkyl)-(C₁-C₆alkyl).

In yet a further embodiment, R² is C₃-C₈ cycloalkyl, C₃-C₈ cycloalkylalkyl, or C₁-C₆alkyl.

In yet another embodiment, R² is C₃-C₈ cycloalkyl.

In an even further embodiment, R² is C₃-C₈ cycloalkylalkyl.

In a still further embodiment, R² is C₁-C₆alkyl.

In still yet an even further embodiment, R² is cyclopropyl, cyclobutyl, cyclopentyl, cyclohexyl, cycloheptyl, cyclohexylmethyl, methyl, isopropyl, tetrahydropyranyl, pyridinyl, or phenyl.

In still yet an even further embodiment, R² is cyclopropyl, cyclobutyl, cyclopentyl, cyclohexyl, cycloheptyl, cyclohexylmethyl, methyl, isopropyl, tetrahydropyran-4-yl, pyridin-2-yl, or phenyl-4-yl.

In yet still another embodiment, R² is cyclohexylmethyl, cyclohexyl, cyclobutyl, cyclopropyl, cycloheptyl, cyclopentyl, methyl, or isopropyl.

In an even further embodiment, R² is cyclohexylmethyl.

In yet an even further embodiment, R² is cyclohexyl.

In yet still an even further embodiment, R² is cyclobutyl.

In another embodiment, R² is cyclopropyl.

In yet another embodiment, R² is cycloheptyl.

In still yet another embodiment, R² is cyclopentyl.

In an even further embodiment, R² is isopropyl.

In still yet a further embodiment, R² is methyl.

In another embodiment, R² is substituted with alkoxy.

In yet another embodiment, R² is substituted with methoxy.

In another embodiment, R¹ is C₆-C₁₄aryl, C₆-C₁₄heteroaryl, (C₆-C₁₄aryl)-(C₁-C₆alkyl), C₃-C₈cycloalkyl, (C₃-C₈heterocycloalkyl)-(C₁-C₆alkyl), C₁-C₆alkyl, (C₆-C₁₄aryl)-(C₁-C₆alkenyl), or C₃-C₈heterocycloalkyl; and R² is C₆-C₁₄aryl, C₆-C₁₄heteroaryl, C₃-C₈cycloalkyl, C₁-C₆alkyl, C₃-C₈heterocycloalkyl, or (C₃-C₈cycloalkyl)-(C₁-C₆alkyl).

In still another embodiment, R¹ is piperidinyl, tetrahydropyranyl, phenylethyl, butyl, phenylallyl, cyclohexyl, tetrahydro-2H-thiopyranyl, morpholinylethyl, phenylmethyl, tetrahydrofuranyl, pyridinyl, methyl, cyclobutyl, phenyl, ethyl, or benzo[d][1,3]dioxolyl; and R² is cyclopropyl, cyclobutyl, cyclopentyl, cyclohexyl, cycloheptyl, cyclohexylmethyl, methyl, isopropyl, tetrahydropyranyl, pyridinyl, or phenyl.

In still another embodiment, R¹ is piperidin-yl-4-yl, tetrahydropyran-4-yl, tetrahydropyran-3-yl, phenylethyl, butyl, phenylallyl, cyclohexyl, tetrahydro-2H-thiopyran-3-yl, N-morpholinylethyl, phenylmethyl, tetrahydrofuran-3-yl, pyridin-2-yl, pyridin-4-yl, pyridin-2-yl-5-yl, pyridin-2-yl-6-yl, methylyl, cyclobutyl, phenyl-4-yl, ethylyl, or benzo[d][1,3]dioxol-5-yl; and is cyclopropyl, cyclobutyl, cyclopentyl, cyclohexyl, cycloheptyl, cyclohexylmethyl, methyl, isopropyl, tetrahydropyran-4-yl, pyridin-2-yl, or phenyl-4-yl.

In yet a further embodiment, R¹ is heterocycloalkyl, alkyl, arylalkenyl, or arylalkyl; and R² is cycloalkyl, cycloalkylalkyl, or alkyl.

In still yet another embodiment, R¹ is heterocycloalkyl, C₁-C₆alkyl, arylalkenyl, or arylalkyl; and R² is C₃-C₈ cycloalkyl, C₃-C₈ cycloalkylalkyl, or C₁-C₆alkyl.

In another embodiment, R¹ is heterocycloalkyl substituted with at least one alkyl, C₁-C₆alkyl substituted with at least one alkyl, arylalkenyl substituted with at least one alkyl, or arylalkyl substituted with at least one alkyl; and R² is C₃-C₈ cycloalkyl, C₃-C₈ cycloalkylalkyl, or C₁-C₆alkyl.

In yet a further embodiment, R¹ is a heterocycloalkyl substituted with at least one methyl.)

In an even further embodiment, R¹ is —C₂-C₃alkyl-R³,

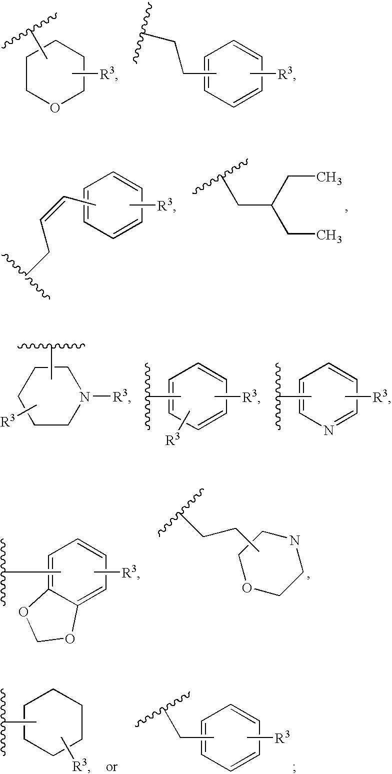

and each R³ is independently selected from H, lower alkyl, cyano, sulfinyl, haloalkyl, amide, alkoxy, halogen, C₁-C₆alkylcarboxy, hydroxy, and amino.

In yet still another embodiment, R¹ is —C₂-C₃alkyl-R³,

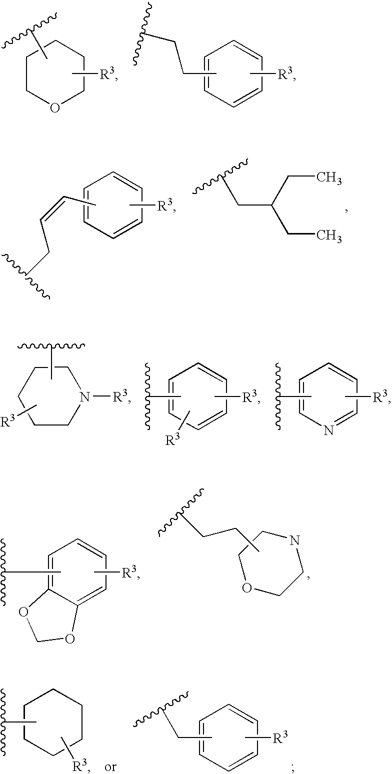

; each R³ is independently selected from H, lower alkyl, cyano, sulfinyl, haloalkyl, amide, alkoxy, halogen, C₁-C₆alkylcarboxy, hydroxy, and amino; R² is

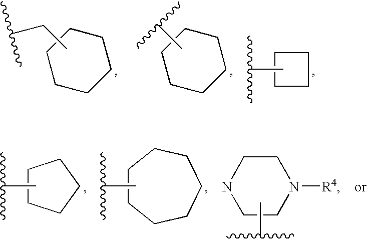

C₁-C₃ alkyl; and R⁴ is a C₃-C₆cycloalkyl.

In a still further embodiment, R² is

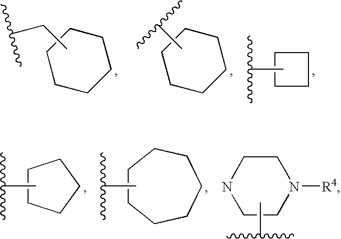

or C₁-C₃ alkyl; and R⁴ is a C₃-C₆cycloalkyl.

In yet another embodiment, R¹ is

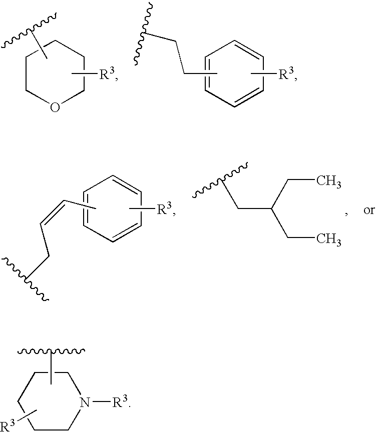

In an even further embodiment, R¹ is

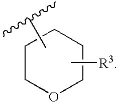

In yet an even further embodiment, R¹ is

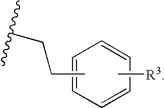

In yet still a further embodiment, R¹ is

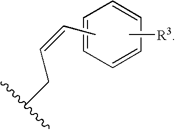

In an even still further embodiment, R¹ is

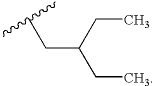

In another embodiment, R¹ is

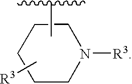

In yet another embodiment, R¹ is —C₂-C₃alkyl-R³.

In still yet another embodiment, R¹ is

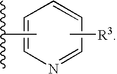

In still even a further embodiment, R¹ is

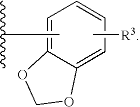

In yet still an even further embodiment, R¹ is

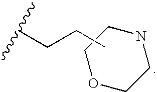

In an even still further embodiment, R¹ is

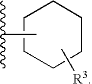

In an even further embodiment, R¹ is

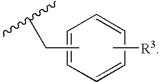

In a further embodiment, R² is

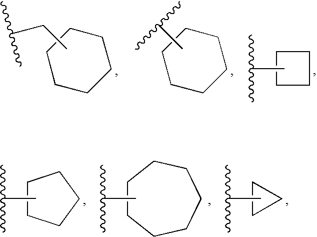

methyl, or isopropyl.

In an even further embodiment, R² is

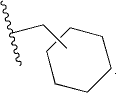

In yet a further embodiment, R² is

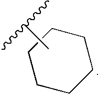

In still a further embodiment, R² is

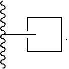

In a still further embodiment, R² is

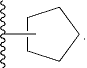

In yet another embodiment, R² is

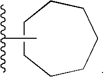

In a further embodiment, R² is

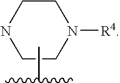

In another embodiment, R² is

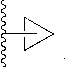

In an even further embodiment, R² is C₁-C₃ alkyl.

In yet a further embodiment, R¹ is

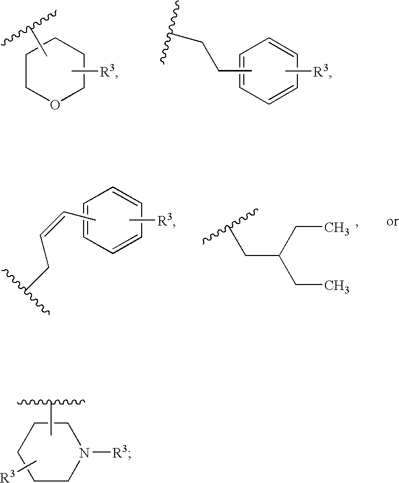

### and R² is

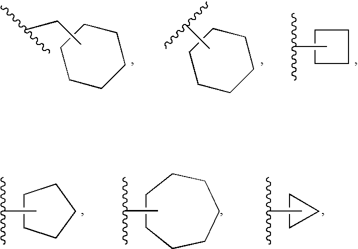

methyl, or isopropyl.

In another embodiment, R³ is independently selected from H, lower alkyl, cyano, sulfinyl, haloalkyl, amide, alkoxy, halogen, C₁-C₆alkylcarboxy, hydroxy, and amino

In still another embodiment, R³ is independently selected from H, CH₃, cyano, —S(═O)CH₃, CF₃, —C(═O)NHCH₃, —OCH₃, Cl, —C(═O)CH₃, OH, and N(CH₃)₂.

In an even further embodiment, R⁴ is a cycloalkyl.

In a still further embodiment, R⁴ is a C₃-C₆cycloalkyl.

Yet an even further embodiment is directed to at least one compound selected from (4-Cyclohexyl-piperazin-1-yl)-[6-(tetrahydro-pyran-4-yl)-6-aza-spiro[2.5]oct-1-yl]-methanone; (4-Cyclohexylmethyl-piperazin-1-yl)-[6-(tetrahydro-pyran-4-yl)-6-aza-spiro[2.5]oct-1-yl]-methanone ; [6-(2-Ethyl-butyl)-6-aza-spiro[2.5]oct-1-yl]-(4-methyl-piperazin-1-yl)-methanone; (4-Methyl-piperazin-1-yl)-[6((E)-3-phenyl-allyl)-6-aza-spiro[2.5]oct-1-yl]-methanone; (4-Methyl-piperazin-1-yl)-(6-phenethyl-6-aza-spiro[2.5]oct-1-yl)-methanone; (4-Methyl-piperazin-1-yl)-[6-(1-methyl-piperidin-4-yl)-6-aza-spiro[2.5]oct-1-yl]-methanone; (4-Methyl-piperazin-1-yl)-[6-(tetrahydro-pyran-4-yl)-6-aza-spiro[2.5]oct-1-yl]-methanone; (4-Cyclobutylpiperazin-1-yl)(6-(tetrahydro-2H-pyran-4-yl)-6-azaspiro[2.5]octan-1-yl)methanone; (4-Cyclopropylpiperazin-1-yl)(6-(tetrahydro-2H-pyran-4-yl)-6-azaspiro[2.5]octan-1-yl)methanone ; (4-Isopropylpiperazin-1-yl)(6-(tetrahydro-2H-pyran-4-yl)-6-azaspiro[2.5]octan-1-yl)methanone; (4-Cycloheptylpiperazin-1-yl)(6-(tetrahydro-2H-pyran-4-yl)-6-azaspiro[2.5]octan-1-yl)methanone; (4-cyclopentylpiperazin-1-yl)(6-(tetrahydro-2H-pyran-4-yl)-6-azaspiro[2.5]octan-1-yl)methanone ; 4-Pyridin-2-yl-piperazin-1-yl)-[6-(tetrahydro-pyran-4-yl)-6-aza-spiro[2.5]oct-1-yl]-methanone; (4-Pyridin-4-yl-piperazin-1-yl)-[6-(tetrahydro-pyran-4-yl)-6-aza-spiro[2.5]oct-1-yl]-methanone; [4-(4-Methoxy-phenyl)-piperazin-1-yl]-[6-(tetrahydro-pyran-4-yl)-6-aza-spiro[2.5]oct-1-yl]-methanone; (4-Cyclohexyl-piperazin-1-yl)-(6-phenethyl-6-aza-spiro[2.5]oct-1-yl)-methanone; (6-Cyclohexyl-6-aza-spiro[2.5]oct-1-yl)-(4-cyclohexyl-piperazin-1-yl)-methanone; (4-Cyclohexyl-piperazin-1-yl)-[6-(1-methyl-piperidin-4-yl)-6-aza-spiro[2.5]oct-1-yl]-methanone; (4-Cyclohexyl-piperazin-1-yl)-(6-isopropyl-6-aza-spiro[2.5]oct-1-yl)-methanone; (6-Cyclobutyl-6-aza-spiro[2.5]oct-1-yl)-[4-(tetrahydro-pyran-4-yl)-piperazin-1-yl]-methanone; (6-Benzyl-6-aza-spiro[2.5]oct-1-yl)-(4-cyclohexyl-piperazin-1-yl)-methanone; [6-(Tetrahydro-pyran-4-yl)-6-aza-spiro[2.5]oct-1-yl]-[4-(tetrahydro-pyran-4-yl)-piperazin-1-yl]-methanone; [6-(2-Benzyloxy-ethyl)-6-aza-spiro[2.5]oct-1-yl]-(4-cyclohexyl-piperazin-1-yl)-methanone; (4-Cyclobutyl-piperazin-1-yl)-[6-(tetrahydro-thiopyran-3-yl)-6-aza-spiro[2.5]oct-1-yl]-methanone; (4-Cyclobutyl-piperazin-1-yl)-(6-cyclohexyl-6-aza-spiro[2.5]oct-1-yl)-methanone; (4-Cyclobutyl-piperazin-1-yl)-[6-(tetrahydro-pyran-3-yl)-6-aza-spiro[2.5]oct-1-yl]-methanone; (4-Cyclobutyl-piperazin-1-yl)-[6-(tetrahydro-furan-3-yl)-6-aza-spiro[2.5]oct-1-yl]-methanone; (4-Cyclobutyl-piperazin-1-yl)-[6-(2-morpholin-4-yl-ethyl)-6-aza-spiro[2.5]oct-1-yl]-methanone; (4-Cyclohexyl-piperazin-1-yl)-(6-pyridin-4-yl-6-aza-spiro[2.5]oct-1-yl)-methanone; [1-(4-Cyclobutyl-piperazine-1-carbonyl)-6-aza-spiro[2.5]oct-6-yl]-acetic acid tert-butyl ester; 2-[1-(4-Cyclobutyl-piperazine-1-carbonyl)-6-aza-spiro[2.5]oct-6-yl]-N-methyl-acetamide; (4-Cyclobutyl-piperazin-1-yl)-[6-(4-methanesulfonyl-phenyl)-6-aza-spiro[2.5]oct-1-yl]-methanone; (4-Cyclobutyl-piperazin-1-yl)-[6-(4-methoxy-phenyl)-6-aza-spiro[2.5]oct-1-yl]-methanone; 4-[1-(4-Cyclobutyl-piperazine-1-carbonyl)-6-aza-spiro[2.5]oct-6-yl]-benzoic acid methyl ester; (4-Cyclobutyl-piperazin-1-yl)-[6-(6-methoxy-pyridin-2-yl)-6-aza-spiro[2.5]oct-1-yl]-methanone; [6-(6-Chloro-pyridin-2-yl)-6-aza-spiro[2.5]oct-1-yl]-(4-cyclobutyl-piperazin-1-yl)-methanone; (4-Cyclohexyl-piperazin-1-yl)-[6-(2-methoxy-ethyl)-6-aza-spiro[2.5]oct-1-yl]-methanone; (4-Cyclobutyl-piperazin-1-yl)-(6-pyridin-2-yl-6-aza-spiro[2.5]oct-1-yl)-methanone; (4-Cyclobutyl-piperazin-1-yl)-(6-p-tolyl-6-aza-spiro[2.5]oct-1-yl)-methanone; 4-Cyclohexyl-piperazin-1-yl)-[6-(5-trifluoromethyl-pyridin-2-yl)-6-aza-spiro[2.5]oct-1-yl]-methanone; (6-Benzo[1,3]dioxol-5-yl-6-aza-spiro[2.5]oct-1-yl)-(4-cyclohexyl-piperazin-1-yl)-methanone; (4-(pyridin-3-yl)piperazin-1-yl)(6-(tetrahydro-2H-pyran-4-yl)-6-azaspiro[2.5]octan-1-yl)methanone; (4-(2-methylpyridin-4-yl)piperazin-1-yl)(6-(tetrahydro-2H-pyran-4-yl)-6-azaspiro[2.5]octan-1-yl)methanone; and (4-(3-methylpyridin-4-yl)piperazin-1-yl)(6-(tetrahydro-2H-pyran-4-yl)-6-azaspiro[2.5]octan-1-yl)methanone; and enantiomers thereof, pharmaceutically acceptable salts thereof, or mixtures thereof.

Another embodiment is directed to at least one compound selected from (4-Cyclohexyl-piperazin-1-yl)-[6-(tetrahydro-pyran-4-yl)-6-aza-spiro[2.5]oct-1-yl]-methanone; (4-Cyclohexylmethyl-piperazin-1-yl)-[6-(tetrahydro-pyran-4-yl)-6-aza-spiro[2.5]oct-1-yl]-methanone; [6-(2-Ethyl-butyl)-6-aza-spiro[2.5]oct-1-yl]-(4-methyl-piperazin-1-yl)-methanone; (4-Methyl-piperazin-1-yl)-[6-((E)-3-phenyl-allyl)-6-aza-spiro[2.5]oct-1-yl]-methanone; (4-Methyl-piperazin-1-yl)-(6-phenethyl-6-aza-spiro[2.5]oct-1-yl)-methanone; (4-Methyl-piperazin-1-yl)-[6-(1-methyl-piperidin-4-yl)-6-aza-spiro[2.5]oct-1-yl]-methanone; (4-Methyl-piperazin-1-yl)-[6-(tetrahydro-pyran-4-yl)-6-aza-spiro[2.5]oct-1-yl]-methanone; (4-Cyclobutylpiperazin-1-yl)(6-(tetrahydro-2H-pyran-4-yl)-6-azaspiro[2.5]octan-1-yl)methanone; (4-Cyclopropylpiperazin-1-yl)(6-(tetrahydro-2H-pyran-4-yl)-6-azaspiro[2.5]octan-1-yl)methanone; (4-Isopropylpiperazin-1-yl)(6-(tetrahydro-2H-pyran-4-yl)-6-azaspiro[2.5]octan-1-yl)methanone; (4-Cycloheptylpiperazin-1-yl)(6-(tetrahydro-2H-pyran-4-yl)-6-azaspiro[2.5]octan-1-yl)methanone; (4-cyclopentylpiperazin-1-yl)(6-(tetrahydro-2H-pyran-4-yl)-6-azaspiro[2.5]octan-1-yl)methanone; 4-Pyridin-2-yl-piperazin-1-yl)-[6-(tetrahydro-pyran-4-yl)-6-aza-spiro[2.5]oct-1-yl]-methanone; (4-Pyridin-4-yl-piperazin-1-yl)-[6-(tetrahydro-pyran-4-yl)-6-aza-spiro[2.5]oct-1-yl]-methanone; [4-(4-Methoxy-phenyl)-piperazin-1-yl]-[6-(tetrahydro-pyran-4-yl)-6-aza-spiro[2.5]oct-1-yl]-methanone; (4-Cyclohexyl-piperazin-1-yl)-(6-phenethyl-6-aza-spiro[2.5]oct-1-yl)-methanone; (6-Cyclohexyl-6-aza-spiro[2.5]oct-1-yl)-(4-cyclohexyl-piperazin-1-yl)-methanone; (4-Cyclohexyl-piperazin-1-yl)-[6-(1-methyl-piperidin-4-yl)-6-aza-spiro[2.5]oct-1-yl]-methanone; (4-Cyclohexyl-piperazin-1-yl)-(6-isopropyl-6-aza-spiro[2.5]oct-1-yl)-methanone; (6-Cyclobutyl-6-aza-spiro[2.5]oct-1-yl)-[4-(tetrahydro-pyran-4-yl)-piperazin-1-yl]-methanone; (6-Benzyl-6-aza-spiro[2.5]oct-1-yl)-(4-cyclohexyl-piperazin-1-yl)-methanone; [6-(Tetrahydro-pyran-4-yl)-6-aza-spiro[2.5]oct-1-yl]-[4-(tetrahydro-pyran-4-yl)-piperazin-1-yl]-methanone; [6-(2-Benzyloxy-ethyl)-6-aza-spiro[2.5]oct-1-yl]-(4-cyclohexyl-piperazin-1-yl)-methanone; (4-Cyclobutyl-piperazin-1-yl)-[6-(tetrahydro-thiopyran-3-yl)-6-aza-spiro[2.5]oct-1-yl]-methanone; (4-Cyclobutyl-piperazin-1-yl)-(6-cyclohexyl-6-aza-spiro[2.5]oct-1-yl)-methanone; (4-Cyclobutyl-piperazin-1-yl)-[6-(tetrahydro-pyran-3-yl)-6-aza-spiro[2.5]oct-1-yl]-methanone; (4-Cyclobutyl-piperazin-1-yl)-[6-(tetrahydro-furan-3-yl)-6-aza-spiro[2.5]oct-1-yl]-methanone; (4-Cyclobutyl-piperazin-1-yl)-[6-(2-morpholin-4-yl-ethyl)-6-aza-spiro[2.5]oct-1-yl]-methanone; (4-Cyclohexyl-piperazin-1-yl)-(6-pyridin-4-yl-6-aza-spiro[2.5]oct-1-yl)-methanone; [1-(4-Cyclobutyl-piperazine-1-carbonyl)-6-aza-spiro[2.5]oct-6-yl]-acetic acid tert-butyl ester; 2-[1-(4-Cyclobutyl-piperazine-1-carbonyl)-6-aza-spiro[2.5]oct-6-yl]-N-methyl-acetamide; (4-Cyclobutyl-piperazin-1-yl)-[6-(4-methanesulfonyl-phenyl)-6-aza-spiro[2.5]oct-1-yl]-methanone; (4-Cyclobutyl-piperazin-1-yl)-[6-(4-methoxy-phenyl)-6-aza-spiro[2.5]oct-1-yl]-methanone; 4-[1-(4-Cyclobutyl-piperazine-1-carbonyl)-6-aza-spiro[2.5]oct-6-yl]-benzoic acid methyl ester; (4-Cyclobutyl-piperazin-1-yl)-[6-(6-methoxy-pyridin-2-yl)-6-aza-spiro[2.5]oct-1-yl]-methanone; [6-(6-Chloro-pyridin-2-yl)-6-aza-spiro[2.5]oct-1-yl]-(4-cyclobutyl-piperazin-1-yl)-methanone; (4-Cyclohexyl-piperazin-1-yl)-[6-(2-methoxy-ethyl)-6-aza-spiro[2.5]oct-1-yl]-methanone; (4-Cyclobutyl-piperazin-1-yl)-(6-pyridin-2-yl-6-aza-spiro[2.5]oct-1-yl)-methanone; (4-Cyclobutyl-piperazin-1-yl)-(6-p-tolyl-6-aza-spiro[2.5]oct-1-yl)-methanone; 4-Cyclohexyl-piperazin-1-yl)-[6-(5-trifluoromethyl-pyridin-2-yl)-6-aza-spiro[2.5]oct-1-yl]-methanone; (6-Benzo[1,3]dioxol-5-yl-6-aza-spiro[2.5]oct-1-yl)-(4-cyclohexyl-piperazin-1-yl)-methanone; (4-(pyridin-3-yl)piperazin-1-yl)(6-(tetrahydro-2H-pyran-4-yl)-6-azaspiro[2.5]octan-1-yl)methanone; (4-(2-methylpyridin-4-yl)piperazin-1-yl)(6-(tetrahydro-2H-pyran-4-yl)-6-azaspiro[2.5]octan-1-yl)methanone; and (4-(3-methylpyridin-4-yl)piperazin-1-yl)(6-(tetrahydro-2H-pyran-4-yl)-6-azaspiro[2.5]octan-1-yl)methanone; and pharmaceutically acceptable salts thereof, or mixtures thereof.

Still another embodiment is directed to at least one compound selected from (4-Cyclohexyl-piperazin-1-yl)-[6-(tetrahydro-pyran-4-yl)-6-aza-spiro[2.5]oct-1-yl]-methanone; (4-Cyclohexylmethyl-piperazin-1-yl)-[6-(tetrahydro-pyran-4-yl)-6-aza-spiro[2.5]oct-1-yl]-methanone; (4-Cyclobutylpiperazin-1-yl)(6-(tetrahydro-2H-pyran-4-yl)-6-azaspiro[2.5]octan-1-yl)methanone; (4-Cyclopropylpiperazin-1-yl)(6-(tetrahydro-2H-pyran-4-yl)-6-azaspiro[2.5]octan-1-yl)methanone; (4-Isopropylpiperazin-1-yl)(6-(tetrahydro-2H-pyran-4-yl)-6-azaspiro[2.5]octan-1-yl)methanone; (4-Cycloheptylpiperazin-1-yl)(6-(tetrahydro-2H-pyran-4-yl)-6-azaspiro[2.5]octan-1-yl)methanone; (4-cyclopentylpiperazin-1-yl)(6-(tetrahydro-2H-pyran-4-yl)-6-azaspiro[2.5]octan-1-yl)methanone; (4-Pyridin-4-yl-piperazin-1-yl)-[6-(tetrahydro-pyran-4-yl)-6-aza-spiro[2.5]oct-1-yl]-methanone; [4-(4-Methoxy-phenyl)-piperazin-1-yl]-[6-(tetrahydro-pyran-4-yl)-6-aza-spiro[2.5]oct-1-yl]-methanone; (4-Cyclohexyl-piperazin-1-yl)-(6-phenethyl-6-aza-spiro[2.5]oct-1-yl)-methanone; (6-Cyclohexyl-6-aza-spiro[2.5]oct-1-yl)-(4-cyclohexyl-piperazin-1-yl)-methanone; (4-Cyclohexyl-piperazin-1-yl)-[6-(1-methyl-piperidin-4-yl)-6-aza-spiro[2.5]oct-1-yl]-methasone; (4-Cyclohexyl-piperazin-1-yl)-(6-isopropyl-6-aza-spiro[2.5]oct-1-yl)-methanone; (6-Cyclobutyl-6-aza-spiro[2.5]oct-1-yl)-[4-(tetrahydro-pyran-4-yl)-piperazin-1-yl]-methanone; (6-Benzyl-6-aza-spiro[2.5]oct-1-yl)-(4-cyclohexyl-piperazin-1-yl)-methanone; [6-(Tetrahydro-pyran-4-yl)-6-aza-spiro[2.5]oct-1-yl]-[4-(tetrahydro-pyran-4-yl)-piperazin-1-yl]-methanone; [6-(2-Benzyloxy-ethyl)-6-aza-spiro[2.5]oct-1-yl]-(4-cyclohexyl-piperazin-1-yl)-methanone; (4-Cyclobutyl-piperazin-1-yl)-[6-(tetrahydro-thiopyran-3-yl)-6-aza-spiro[2.5]oct-1-yl]-methanone; (4-Cyclobutyl-piperazin-1-yl)-(6-cyclohexyl-6-aza-spiro[2.5]oct-1-yl)-methanone; (4-Cyclobutyl-piperazin-1-yl)-[6-(tetrahydro-pyran-3-yl)-6-aza-spiro[2.5]oct-1-yl]-methanone; (4-Cyclobutyl-piperazin-1-yl)-[6-(tetrahydro-furan-3-yl)-6-aza-spiro[2.5]oct-1-yl]-methanone; (4-Cyclobutyl-piperazin-1-yl)-[6-(2-morpholin-4-yl-ethyl)-6-aza-spiro[2.5]oct-1-yl]-methanone; (4-Cyclohexyl-piperazin-1-yl)-(6-pyridin-4-yl-6-aza-spiro[2.5]oct-1-yl)-methanone; [1-(4-Cyclobutyl-piperazine-1-carbonyl)-6-aza-spiro[2.5]oct-6-yl]-acetic acid tert-butyl ester; 2-[1-(4-Cyclobutyl-piperazine-1-carbonyl)-6-aza-spiro[2.5]oct-6-yl]-N-methyl-acetamide; (4-Cyclobutyl-piperazin-1-yl)-[6-(4-methanesulfonyl-phenyl)-6-aza-spiro[2.5]oct-1-yl]-methanone; (4-Cyclobutyl-piperazin-1-yl)-[6-(4-methoxy-phenyl)-6-aza-spiro[2.5]oct-1-yl]-methanone; 4-[1-(4-Cyclobutyl-piperazine-1-carbonyl)-6-aza-spiro[2.5]oct-6-yl]-benzoic acid methyl ester; (4-(pyridin-3-yl)piperazin-1-yl)(6-(tetrahydro-2H-pyran-4-yl)-6-azaspiro[2.5]octan-1-yl)methanone; (4-(2-methylpyridin-4-yl)piperazin-1-yl)(6-(tetrahydro-2H-pyran-4-yl)-6-azaspiro[2.5]octan-1-yl)methanone; and (4-(3-methylpyridin-4-yl)piperazin-1-yl)(6-(tetrahydro-2H-pyran-4-yl)-6-azaspiro[2.5]octan-1-yl)methanone; and enantiomers thereof, pharmaceutically acceptable salts thereof, or mixtures thereof.

Even still another embodiment is directed to at least one compound selected from (4-Cyclohexyl-piperazin-1-yl)-[6-(tetrahydro-pyran-4-yl)-6-aza-spiro[2.5]oct-1-yl]-methanone; (4-Cyclohexylmethyl-piperazin-1-yl)-[6-(tetrahydro-pyran-4-yl)-6-aza-spiro[2.5]oct-1-yl]-methanone; (4-Cyclobutylpiperazin-1-yl)(6-(tetrahydro-2H-pyran-4-yl)-6-azaspiro[2.5]octan-1-yl)methanone; (4-Cyclopropylpiperazin-1-yl)(6-(tetrahydro-2H-pyran-4-yl)-6-azaspiro[2.5]octan-1-yl)methanone; (4-Isopropylpiperazin-1-yl)(6-(tetrahydro-2H-pyran-4-yl)-6-azaspiro[2.5]octan-1-yl)methanone; (4-Cycloheptylpiperazin-1-yl)(6-(tetrahydro-2H-pyran-4-yl)-6-azaspiro[2.5]octan-1-yl)methanone; (4-cyclopentylpiperazin-1-yl)(6-(tetrahydro-2H-pyran-4-yl)-6-azaspiro[2.5]octan-1-yl)methanone; (4-Pyridin-4-yl-piperazin-1-yl)-[6-(tetrahydro-pyran-4-yl)-6-aza-spiro[2.5]oct-1-yl]-methanone; [4-(4-Methoxy-phenyl)-piperazin-1-yl]-[6-(tetrahydro-pyran-4-yl)-6-aza-spiro[2.5]oct-1-yl]-methanone; (4-Cyclohexyl-piperazin-1-yl)-(6-phenethyl-6-aza-spiro[2.5]oct-1-yl)-methanone; (6-Cyclohexyl-6-aza-spiro[2.5]oct-1-yl)-(4-cyclohexyl-piperazin-1-yl)-methanone; (4-Cyclohexyl-piperazin-1-yl)-[6-(1-methyl-piperidin-4-yl)-6-aza-spiro[2.5]oct-1-yl]-methanone; (4-Cyclohexyl-piperazin-1-yl)-(6-isopropyl-6-aza-spiro[2.5]oct-1-yl)-methanone; (6-Cyclobutyl-6-aza-spiro[2.5]oct-1-yl)-[4-(tetrahydro-pyran-4-yl)-piperazin-1-yl]-methanone; (6-Benzyl-6-aza-spiro[2.5]oct-1-yl)-(4-cyclohexyl-piperazin-1-yl)-methanone; [6-(Tetrahydro-pyran-4-yl)-6-aza-spiro[2.5]oct-1-yl]-[4-(tetrahydro-pyran-4-yl)-piperazin-1-yl]-methanone; [6-(2-Benzyloxy-ethyl)-6-aza-spiro[2.5]oct-1-yl]-(4-cyclohexyl-piperazin-1-yl)-methanone; (4-Cyclobutyl-piperazin-1-yl)-[6-(tetrahydro-thiopyran-3-yl)-6-aza-spiro[2.5]oct-1-yl]-methanone; (4-Cyclobutyl-piperazin-1-yl)-(6-cyclohexyl-6-aza-spiro[2.5]oct-1-yl)-methanone; (4-Cyclobutyl-piperazin-1-yl)-[6-(tetrahydro-pyran-3-yl)-6-aza-spiro[2.5]oct-1-yl]-methanone; (4-Cyclobutyl-piperazin-1-yl)-[6-(tetrahydro-furan-3-yl)-6-aza-spiro[2.5]oct-1-yl]-methanone; (4-Cyclobutyl-piperazin-1-yl)-[6-(2-morpholin-4-yl-ethyl)-6-aza-spiro[2.5]oct-1-yl]-methanone; (4-Cyclohexyl-piperazin-1-yl)-(6-pyridin-4-yl-6-aza-spiro[2.5]oct-1-yl)-methanone; [1-(4-Cyclobutyl-piperazine-1-carbonyl)-6-aza-Spiro[2.5]oct-6-yl]-acetic acid tert-butyl ester; 2-[1-(4-Cyclobutyl-piperazine-1-carbonyl)-6-aza-spiro[2.5]oct-6-yl]-N-methyl-acetamide; (4-Cyclobutyl-piperazin-1-yl)-[6-(4-methanesulfonyl-phenyl)-6-aza-spiro[2.5]oct-1-yl]-methanone; (4-Cyclobutyl-piperazin-1-yl)-[6-(4-methoxy-phenyl)-6-aza-spiro[2.5]oct-1-yl]-methanone; 4-[1-(4-Cyclobutyl-piperazine-1-carbonyl)-6-aza-spiro[2.5]oct-6-yl]-benzoic acid methyl ester; (4-(pyridin-3-yl)piperazin-1-yl)(6-(tetrahydro-2H-pyran-4-yl)-6-azaspiro[2.5]octan-1-yl)methanone; (4-(2-methylpyridin-4-yl)piperazin-1-yl)(6-(tetrahydro-2H-pyran-4-yl)-6-azaspiro[2.5]octan-1-yl)methanone; and (4-(3-methylpyridin-4-yl)piperazin-1-yl)(6-(tetrahydro-2H-pyran-4-yl)-6-azaspiro[2.5]octan-1-yl)methanone; and pharmaceutically acceptable salts thereof, or mixtures thereof.

In yet another embodiment is directed to at least one compound selected from (4-Isopropylpiperazin-1-yl)(6-(tetrahydro-2H-pyran-4-yl)-6-azaspiro[2.5]octan-1-yl)methanone; 4-Cyclohexyl-piperazin-1-yl)-(6-phenethyl-6-aza-spiro[2.5]oct-1-yl)-methanone; and (4-Cyclobutyl-piperazin-1-yl)-(6-cyclohexyl-6-aza-spiro[2.5]oct-1-yl)-methanone; and enantiomers thereof, pharmaceutically acceptable salts thereof, or mixtures thereof.

It will be understood that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of the compounds of formula I. The optically active forms of the compound of the invention may be prepared, for example, by chiral chromatographic separation of a racemate, by synthesis from optically active starting materials or by asymmetric synthesis based on the procedures described thereafter.

It will also be appreciated that certain compounds of the invention may exist as geometrical isomers, for example E and Z isomers of alkenes. The present invention includes any geometrical isomer of the compounds of formula I. It will further be understood that the present invention encompasses tautomers of the compounds of formula I.

It will also be understood that certain compounds of the invention may exist in solvated, for example hydrated, as well as unsolvated forms. It will further be understood the present invention encompasses all such solvated forms of the compounds of formula I.

The compounds of formula I can also form salts. As a result, when a compound of formula I is referred to herein, such reference includes, unless otherwise indicated, salts thereof. In one embodiment, the compounds of formula I form pharmaceutically acceptable salts. In another embodiment, the compounds of formula I form salts that can, for example, be used to isolate and/or purify the compounds of formula I.

Generally, pharmaceutically acceptable salts of a compound in accordance with formula I can be obtained by using standard procedures well known in the art. These standard procedures include, but are not limited to, for example, the reacting of a sufficiently basic compound, such as, for example, an alkyl amine with a suitable acid, such as, for example, HCl or acetic acid, to afford a physiologically acceptable anion. It may also be possible to make a corresponding alkali metal (such as sodium, potassium, or lithium) or an alkaline earth metal (such as a calcium) salt by treating a compound in accordance with formula I having a suitably acidic proton, such as, for example, a carboxylic acid or a phenol with one equivalent of an alkali metal or alkaline earth metal hydroxide or alkoxide (such as, for example, an ethoxide or methoxide), or a suitably basic organic amine (such as, for example, a choline or meglumine) in an aqueous medium, followed by conventional purification techniques.

In one embodiment, a compound in accordance with formula I may be converted to a pharmaceutically acceptable salt or solvate thereof, particularly, an acid addition salt, such as, for example, hydrochloride, hydrobromide, phosphate, acetate, fumarate, maleate, tartrate, citrate, methanesulphonate, and p-toluenesulphonate.

In general, the compounds of formula I can be prepared in accordance with the following Schemes and the general knowledge of one skilled in the art and/or in accordance with the methods set forth in the Examples that follow. Solvents, temperatures, pressures, and other reaction conditions may readily be selected by one of ordinary skill in the art. Starting materials are commercially available or readily prepared by one skilled in the art. Combinatorial techniques can be employed in the preparation of compounds, for example, where the intermediates possess groups suitable for these techniques. In the schemes, the groups R¹ and R² are as defined hereinabove and P is an amino-protecting group.

The term “amino-protecting group” refers to art-recognized moieties capable of attaching to an amino group so as to prevent the amino group from taking place in reactions occurring elsewhere on the molecule to which the amino group is attached. Acceptable amino-protecting groups, include but are not limited to, for example, amino-protecting groups described in “Protective Groups in Organic Synthesis”, 2nd edition, John Wiley & Sons, 1981. The amino-protecting group may be, for example, a urethane type protective group (which is also referred to as a carbamate protective group), which includes but is not limited to, for example, arylalkyloxycarbonyl groups, such as, for example, benzyloxycarbonyl; and alkoxycarbonyl groups, such as, for example, methoxycarbonyl and tert-butoxycarbonyl. Typically, the amino-protecting group is tert-butoxycarbonyl.

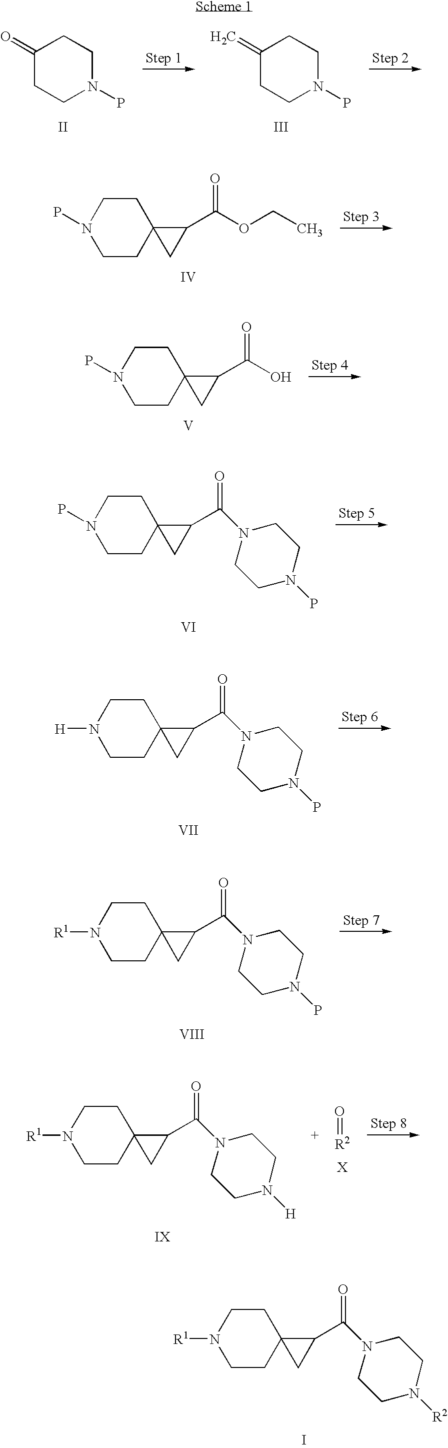

### Step 1

A compound in accordance with formula III can be obtained by treating an appropriate phosphonium salt of triphenyl phosphine and an alkyl halide such as, for example, bromomethane and an appropriate base, such as, for example, n-butyllithium in an appropriate solvent, such as, for example, tetrahydrofuran followed by the addition of a compound in accordance with formula II.

### Step 2

A compound in accordance with formula IV can be obtained by treating a compound in accordance with formula III with an appropriately substituted cyclopropanating reagent, such as, for example, ethyl diazoacetate in the presence of an appropriate metal salt, such as, for example, cuprous cyanide in an appropriate solvent, such as, for example, methylene chloride.

### Step 3

A compound in accordance with formula V can be obtained by hydrolyzing a compound in accordance with formula IV to the corresponding carboxylic acid with an appropriate base, such as, for example, sodium hydroxide or lithium hydroxide in an appropriate solvent, such as, for example, THF, MeOH, water; followed by acidification with an appropriate acid such as, for example, 1N hydrochloric acid.

### Step 4

A compound in accordance with formula VI can be obtained by the treating a compound in accordance with formula V with an appropriate coupling reagent, such as, for example O-(7-azabenzotriazole-1-yl)-N,N,N′N′-tetramethyluronium hexafluorophosphate with an appropriate base, such as, for example, N-ethyldiisopropylamine followed by treating with an appropriately functionalized primary or secondary amine, such as, for example, tert-butyl piperazine-1-carboxylate, in an appropriate solvent, such as, for example, acetonitrile.

### Step 5

A compound in accordance with formula VII can be obtained by treating a compound in accordance with formula VI with an appropriate supported metal catalyst, such as, for example, 10% palladium on carbon in the presence of an appropriate hydrogen donor, such as, for example, hydrogen gas in an appropriate solvent, such as, for example, ethanol.

### Step 6

A compound in accordance with formula VIII can be obtained by treating a compound in accordance with formula VII with an appropriately functionalized aldehyde or ketone in the presence of an appropriate borohydride reagent, such as, for example, sodium cyanoborohydride in the presence of a catalytic quantity of acetic acid in an appropriate solvent, such as, for example, ethanol at elevated temperatures.

### Step 7

A compound in accordance with formula IX can be obtained by treating a compound in accordance with formula VIII with appropriate acid, such as, for example, trifluoroacetic acid in an appropriate solvent, such as, for example, methylene chloride.

### Step 8

A compound in accordance with formula I can be obtained by treating a compound in accordance with formula IX with an appropriately functionalized aldehyde or ketone, such as, for example, a compound in accordance with formula X in the presence of an appropriate borohydride reagent, such as, for example, sodium cyanoborohydride in the presence of a catalytic quantity of acetic acid in an appropriate solvent, such as, for example, ethanol at elevated temperatures.

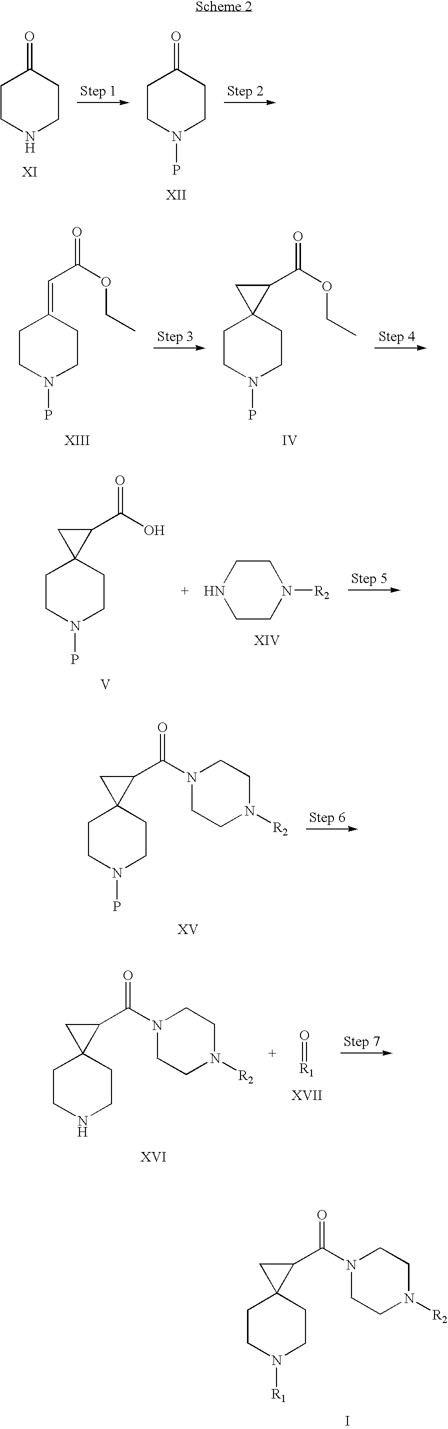

### Step 1

A compound in accordance with formula XII can be obtained by treating a compound in accordance with formula XI with an appropriate protecting group precursor, such as, for example, benzyl chloroformate in the presence of an appropriate base, such as sodium bicarbonate, in an appropriate solvent such as dioxane and water.

### Step 2

A compound in accordance with formula XIII can be obtained by treating a compound in accordance with formula XII with an appropriate phosphorous ylide, such as, for example, (ethoxycarbonylmethylene)triphenylphosphorane or one prepared from ethyl 2-(diethoxyphosphoryl)acetate with an appropriate base, such as, for example, nBuLi in an appropriate solvent such as toluene or THF depending on the reagents used.

### Step 3

A compound in accordance with formula IV can be obtained by treating a compound in accordance with formula XIII with an appropriate sulfur ylide, such as, for example, one prepared from trimethylsulfoxonium iodide by using an appropriate base such as for example, potassium tert-butoxide or sodium hydride, in an appropriate solvent such as for example, DMSO.

### Step 4

A compound in accordance with formula V can be obtained by hydrolyzing a compound in accordance with formula IV to the corresponding carboxylic acid with an appropriate base, such as, for example, sodium hydroxide or lithium hydroxide in an appropriate solvent, such as, for example, THF, MeOH, water; followed by acidification with an appropriate acid such as, for example, 1N hydrochloric acid.

### Step 5

A compound in accordance with formula XV can be obtained by the treating a compound in accordance with formula V with an appropriate coupling reagent, such as, for example HATU or HBTU with an appropriate base, such as, for example, N-ethyldiisopropylamine followed by treating with an appropriately functionalized primary or secondary amine in accordance with formula XIV, such as, for example, 1-cyclohexylpiperazine in an appropriate solvent, such as, for example, DMF.

### Step 6

A compound in accordance with formula XVI can be obtained by treating a compound in accordance with formula XV with an appropriate supported metal catalyst, such as, for example, 10% palladium on carbon in the presence of an appropriate hydrogen donor, such as, for example, hydrogen gas in an appropriate solvent, such as, for example, ethanol or methanol. Alternately, Step 7 can be combined with Step 6 in which the appropriately functionalized formula XVII aldehyde or ketone from Step 7 is incorporated into the Step 6 reaction conditions and is transformed with the reducing agent being an appropriate metal catalyst, such as, for example, 10% palladium on carbon in the presence of an appropriate hydrogen donor, such as, for example, hydrogen gas in an appropriate solvent, such as, for example, ethanol or methanol.

### Step 7

A compound in accordance with formula I can be obtained by treating a compound in accordance with formula XVI with an appropriately functionalized aldehyde or ketone in accordance with formula XVII in the presence of an appropriate reducing reagent, such as, for example, sodium triacetoxyborohydride in an appropriate solvent, such as, for example, DCE.

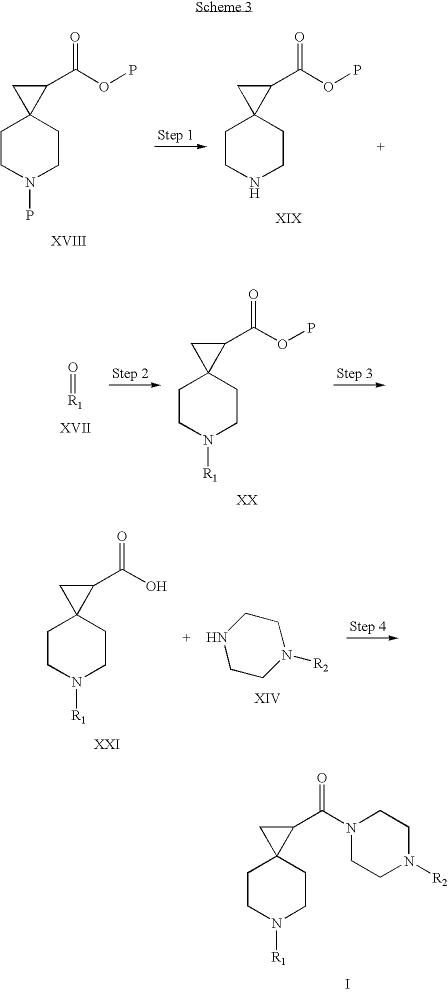

### Step 1

A compound in accordance with formula XIX can be obtained by treating a compound in accordance with formula XVIII with an appropriate supported metal catalyst, such as, for example, 5% palladium on carbon in the presence of an appropriate hydrogen donor, such as, for example, hydrogen gas in an appropriate solvent, such as, for example, ethanol.

### Step 2

A compound in accordance with formula XX can be obtained by treating a compound in accordance with formula XIX with an appropriate ketone or aldehyde in accordance with formula XVII in the presence of an appropriate borohydride reagent, such as, for example, sodium triacetoxyborohydride with and without the presence of catalytic acid in an appropriate solvent, such as, for example, dichloroethane.

### Step 3

A compound in accordance with formula XXI can be obtained by hydrolyzing a compound in accordance with formula XX to the corresponding carboxylic acid under standard conditions using a variety of bases, such as, for example, lithium and sodium potassium hydroxide in any alcoholic or ethereal solvent, such as, for example, THF and MeOH, with and without water. This can be followed by acidification with an appropriate acid, such as, for example, 1-6N hydrochloric acid.

### Step 4

A compound in accordance with formula I can be obtained by treating a compound in accordance with formula XXI with an appropriate coupling reagent, such as, for example HBTU with an appropriate base, such as, for example, N-ethyldiisopropylamine followed by treatment with an appropriately functionalized primary or secondary amine, such as, for example, a compound in accordance with formula XIV in an appropriate solvent, such as, for example, DMF.

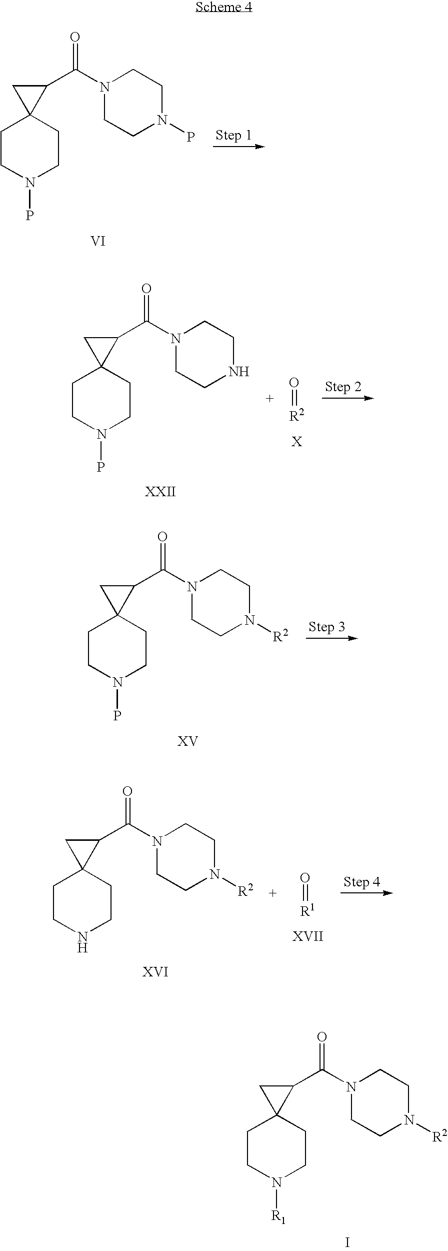

### Step 1

A compound in accordance with formula XXII can be obtained by treating a compound in accordance with formula VI with an appropriate acid, such as, for example, trifluoroacetic acid in an appropriate solvent, such as, for example, methylene chloride.

### Step 2

A compound in accordance with formula XV can be obtained by treating a compound in accordance with formula XXII with an appropriately functionalized aldehyde or ketone in accordance with formula X in the presence of an appropriate reducing reagent, such as, for example, sodium triacetoxyborohydride in an appropriate solvent, such as, for example, DCE.

### Step 3

A compound in accordance with formula XVI can be obtained by treating a compound in accordance with formula XV with an appropriate supported metal catalyst, such as, for example, 5% palladium on carbon in the presence of an appropriate hydrogen donor, such as, for example, hydrogen gas in an appropriate solvent, such as, for example, ethanol or methanol. Alternately, Step 4 can be combined with Step 3 in which the appropriately functionalized formula XVII aldehyde or ketone of Step 4 can be incorporated into the Step 3 reaction conditions and undergo the same transformation with the reducing agent being an appropriate metal catalyst such as for example 10% palladium on carbon in the presence of an appropriate hydrogen donor, such as, for example, hydrogen gas in an appropriate solvent, such as, for example, ethanol or methanol.

### Step 4

A compound in accordance with formula I can be obtained by treating a compound in accordance with formula XVI with an appropriately functionalized aldehyde or ketone in accordance with formula XVII in the presence of an appropriate reducing reagent, such as, for example, sodium triacetoxyborohydride in an appropriate solvent, such as, for example, DCE.

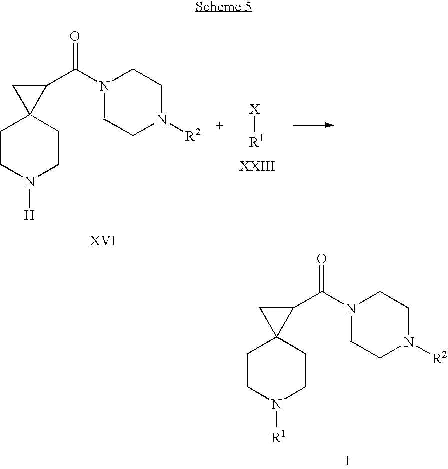

wherein X is a halogen and R¹ is as described hereinabove.

### Step 1

A compound in accordance with formula I can be obtained by treating a compound in accordance with formula XVI with an appropriately functionalized aryl or alkyl halide in accordance with formula XXIII in the presence of an appropriate base such as, for example, triethylamine, K₂CO₃, or lithium bis(trimethylsilyl)amide and in an appropriate solvent, such as, for example, ACN, DMSO, toluene, or THF, with or, in some cases, without an appropriate catalyst, such as, for example, tetrabutylammonium iodide, lithium iodide, or trans-dichlorobis(tri-o-tolyl phosphine)palladium(II).

At least one compound in accordance with formula I may be used to treat a wide range of conditions or disorders in which interacting with the histamine H3 receptor is beneficial. At least one formula I compound may, for example, be useful to treat diseases of the central nervous system, the peripheral nervous system, the cardiovascular system, the pulmonary system, the gastrointestinal system, or the endocrinological system.

In one embodiment, at least one compound of formula I modulates at least one histamine H3 receptor.

The terms “modulate”, “modulates”, “modulating”, or “modulation”, as used herein, refer to, for example, the activation (e.g., agonist activity) or inhibition (e.g., antagonist activity) of at least one histamine H3 receptor.

Another embodiment provides a method for treating a disorder in which modulating the function of at least one histamine H3 receptor is beneficial comprising administering to a warm-blooded animal in need of such treatment a therapeutically effective amount of at least one compound according to formula I.

In yet another embodiment, at least one compound in accordance with formula I may be used as a medicament.

At least one compound in accordance with formula I may be useful to treat at least one autoimmune disorder. Exemplary autoimmune disorders include, but are not limited to, for example, arthritis, skin grafts, organ transplants and similar surgical needs, collagen diseases, various allergies, tumors and viruses.

At least one compound in accordance with formula I may be useful to treat at least one psychiatric disorder. Exemplary psychiatric disorders include, but are not limited to, for example, Psychotic Disorder(s) and Schizophrenia Disorder(s), such as, for example, Schizoaffective Disorder(s), Delusional Disorder(s), Brief Psychotic Disorder(s), Shared Psychotic Disorder(s), and Psychotic Disorder(s) Due to a General Medical Condition; Dementia and other Cognitive Disorder(s); Anxiety Disorder(s), such as, for example, Panic Disorder(s) Without Agoraphobia, Panic Disorder(s) With Agoraphobia, Agoraphobia Without History of Panic Disorder(s), Specific Phobia, Social Phobia, Obsessive-Compulsive Disorder(s), Stress related Disorder(s), Posttraumatic Stress Disorder(s), Acute Stress Disorder(s), Generalized Anxiety Disorder(s) and Generalized Anxiety Disorder(s) Due to a General Medical Condition; Mood Disorder(s), such as, for example, a) Depressive Disorder(s) (including but not limited to, for example, Major Depressive Disorder(s) and Dysthymic Disorder(s)), b) Bipolar Depression and/or Bipolar mania, such as, for example, Bipolar I (which includes, but is not limited to those with manic, depressive or mixed episodes), and Bipolar II, c) Cyclothymiac's Disorder(s), and d) Mood Disorder(s) Due to a General Medical Condition; Sleep Disorder(s), such as, for example, narcolepsy; Disorder(s) Usually First Diagnosed in Infancy, Childhood, or Adolescence including, but not limited to, for example, Mental Retardation, Downs Syndrome, Learning Disorder(s), Motor Skills Disorder(s), Communication Disorders(s), Pervasive Developmental Disorder(s), Attention-Deficit and Disruptive Behavior Disorder(s), Feeding and Eating Disorder(s) of Infancy or Early Childhood, Tic Disorder(s), and Elimination Disorder(s); Substance-Related Disorder(s) including, but not limited to, for example, Substance Dependence, Substance Abuse, Substance Intoxication, Substance Withdrawal, Alcohol-Related Disorder(s), Amphetamines (or Amphetamine-Like)-Related Disorder(s), Caffeine-Related Disorder(s), Cannabis-Related Disorder(s), Cocaine-Related Disorder(s), Hallucinogen-Related Disorder(s), Inhalant-Related Disorder(s), Nicotine-Related Disorder(s)s, Opiod-Related Disorder(s)s, Phencyclidine (or Phencyclidine-Like)-Related Disorder(s), and Sedative-, Hypnotic- or Anxiolytic-Related Disorder(s); Attention-Deficit and Disruptive Behavior Disorder(s); Personality Disorder(s) including, but not limited to, for example, Obsessive-Compulsive Personality Disorder(s); Impulse-Control Disorder(s); Tic Disorders including, but not limited to, for example Tourette's Disorder, Chronic motor or vocal tic disorder; and Transient Tic Disorder.

At least one of the above psychiatric disorders is defined in, for example, the American Psychiatric Association: Diagnostic and Statistical Manual of Mental Disorders, Fourth Edition, Text Revision, Washington, D.C., American Psychiatric Association, 2000.

At least one compound in accordance with formula I may be useful i) to treat obesity or being overweight (e.g., promotion of weight loss and maintenance of weight loss), eating disorders (e.g., binge eating, anorexia, bulimia and compulsive), and/or cravings (for drugs, tobacco, alcohol, any appetizing macronutrients or non-essential food items); ii) to prevent weight gain (e.g., medication-induced or subsequent to cessation of smoking); and/or iii) to modulate appetite and/or satiety.

At least one compound in accordance with formula I may be suitable for treating obesity by reducing appetite and body weight and/or maintaining weight reduction and preventing rebound.

At least one compound in accordance with formula I may be used to prevent or reverse medication-induced weight gain, e.g. weight gain caused by antipsychotic (neuroleptic) treatment(s); and/or weight gain associated with smoking cessation.

At least one compound in accordance with formula I may be useful to treat at least one Neurodegenerative Disorder. Exemplary Neurodegenerative Disorders include, but are not limited to, for example, Alzheimer's Disease (AD); Dementia, which includes, but is not limited to, for example, Alzheimer's Disease (AD), Down syndrome, vascular dementia, Parkinson's Disease (PD), postencephelatic parkinsonism, dementia with Lewy bodies, HIV dementia, Huntington's Disease, amyotrophic lateral sclerosis (ALS), motor neuron diseases (MND), Frontotemporal dementia Parkinson's Type (FTDP), progressive supranuclear palsy (PSP), Pick's Disease, Niemann-Pick's Disease, corticobasal degeneration, traumatic brain injury (TBI), dementia pugilistica, Creutzfeld-Jacob Disease and prion diseases; Cognitive Deficit in Schizophrenia (CDS); Mild Cognitive Impairment (MCI); Age-Associated Memory Impairment (AAMI); Age-Related Cognitive Decline (ARCD); Cognitive Impairment No Dementia (CIND); Multiple Sclerosis; Parkinson's Disease (PD); postencephalitic parkinsonism; Huntington's Disease; amyotrophic lateral sclerosis (ALS); motor neuron diseases (MND); Multiple System Atrophy (MSA); Corticobasal Degeneration; Progressive Supranuclear Paresis; Guillain-Barré Syndrome (GBS); and Chronic Inflammatory Demyelinating Polyneuropathy (CIDP).

At least one compound in accordance with formula I may be useful to treat at least one Neuroinflammatory Disorder including, but not limited to, for example, Multiple Sclerosis (MS), which includes, but is not limited to, for example, Relapse Remitting Multiple Sclerosis (RRMS), Secondary Progressive Multiple Sclerosis (SPMS), and Primary Progressive Multiple Sclerosis (PPMS); Parkinson's disease; Multiple System Atrophy (MSA); Corticobasal Degeneration; Progressive Supranuclear Paresis; Guillain-Barré Syndrome (GBS); and chronic inflammatory demyelinating polyneuropathy (CIDP).

At least one compound in accordance with formula I may be useful to treat at least one Attention-Deficit and Disruptive Behavior Disorder. Exemplary Attention-Deficit and Disruptive Behavior Disorders include, but are not limited to, for example, attention deficit disorder (ADD), attention deficit hyperactivity disorder (ADHD), and affective disorders.

At least one compound in accordance with formula I may be useful to treat pain; acute and chronic pain disorders including but not limited to, for example, Widespread pain, Localized pain, Nociceptive pain, Inflammatory pain, Central pain, Central and peripheral neuropathic pain, Central and peripheral neurogenic pain, Central and peripheral neuralgia, Low back pain, Postoperative pain, Visceral pain, and Pelvic pain; Allodynia; Anesthesia dolorosa; Causalgia; Dysesthesia; Fibromyalgia; Hyperalgesia; Hyperesthesia; Hyperpathia; Ischemic pain; Sciatic pain; Pain associated with cystitis including, but not limited to, interstitial cystitis; Pain associated with multiple sclerosis; Pain associated with arthritis; Pain associated with osteoarthritis; Pain associated with rheumatoid arthritis; and Pain associated with cancer.

In another embodiment, at least one compound in accordance with formula I may be used for the manufacture of a medicament for the treatment of at least one autoimmune, psychiatric, neurodegenerative, neuroinflammatory, or Attention-Deficit and Disruptive Behaviour Disorder described hereinabove.

In yet another embodiment, at least one compound in accordance with formula I may be used for the manufacture of a medicament for the treatment of at least one disorder selected from cognitive deficient in schizophrenia, narcolepsy, obesity, and Alzheimer's disease.

Yet another embodiment provides a method for treating at least one autoimmune, psychiatric, neurodegenerative, neuroinflammatory, or Attention-Deficit and Disruptive Behavior Disorder in a warm-blooded animal, comprising administering to said animal in need of such treatment a therapeutically effective amount of at least one compound according to formula I.

Yet an even further embodiment provides a method for treating at least one disorder selected from cognitive deficient in schizophrenia, narcolepsy, obesity, and alzheimer's disease in a warm-blooded animal, comprising administering to said animal in need of such treatment a therapeutically effective amount of at least one compound according to formula I.

A still further embodiment provides a method for treating cognitive deficient in schizophrenia in a warm-blooded animal, comprising administering to said animal in need of such treatment a therapeutically effective amount of at least one compound according to formula I.

A still yet further embodiment provides a method for treating obesity in a warm-blooded animal, comprising administering to said animal in need of such treatment a therapeutically effective amount of at least one compound according to formula I.

An even still further embodiment provides a method for treating narcolepsy in a warm-blooded animal, comprising administering to said animal in need of such treatment a therapeutically effective amount of at least one compound according to formula I.

A yet still further embodiment provides a method for treating alzheimer's disease in a warm-blooded animal, comprising administering to said animal in need of such treatment a therapeutically effective amount of at least one compound according to formula I.

A still further embodiment provides a method for treating pain in a warm-blooded animal, comprising administering to said animal in need of such treatment a therapeutically effective amount of at least one compound according to formula I.

In one embodiment, the warm-blooded animal is a mammalian species including, but not limited to, for example, humans and domestic animals, such as, for example, dogs, cats, and horses.

In a further embodiment, the warm-blooded animal is a human.

Yet a further embodiment provides the use of a compound in accordance with formula I in therapy.

Yet an even further embodiment provides the use of a compound of formula I in the manufacture of a medicament for use in therapy.

As used herein, the term “therapy” also includes “prophylaxis” unless specifically indicated to the contrary.

In yet another embodiment a compound in accordance with formula I, or a pharmaceutical composition or formulation comprising at least one compound of formula I may be administered concurrently, simultaneously, sequentially or separately with at least one other pharmaceutically active compound selected from the following:

(i) antidepressants, such as, for example, agomelatine, amitriptyline, amoxapine, bupropion, citalopram, clomipramine, desipramine, doxepin duloxetine, elzasonan, escitalopram, fluvoxamine, fluoxetine, gepirone, imipramine, ipsapirone, maprotiline, nortriptyline, nefazodone, paroxetine, phenelzine, protriptyline, ramelteon, reboxetine, robalzotan, sertraline, sibutramine, thionisoxetine, tranylcypromaine, trazodone, trimipramine, venlafaxine and equivalents and pharmaceutically active isomer(s) and metabolite(s) thereof;

(ii) atypical antipsychotics including for example quetiapine and pharmaceutically active isomer(s) and metabolite(s) thereof;

(iii) antipsychotics, such as, for example, amisulpride, aripiprazole, asenapine, benzisoxidil, bifeprunox, carbamazepine, clozapine, chlorpromazine, debenzapine, divalproex, duloxetine, eszopiclone, haloperidol, iloperidone, lamotrigine, loxapine, mesoridazine, olanzapine, paliperidone, perlapine, perphenazine, phenothiazine, phenylbutylpiperidine, pimozide, prochlorperazine, risperidone, sertindole, sulpiride, suproclone, suriclone, thioridazine, trifluoperazine, trimetozine, valproate, valproic acid, zopiclone, zotepine, ziprasidone and equivalents and pharmaceutically active isomer(s) and metabolite(s) thereof;

(iv) anxiolytics, such as, for example, alnespirone, azapirones, benzodiazepines, barbiturates such as adinazolam, alprazolam, balezepam, bentazepam, bromazepam, brotizolam, buspirone, clonazepam, clorazepate, chlordiazepoxide, cyprazepam, diazepam, diphenhydramine, estazolam, fenobam, flunitrazepam, flurazepam, fosazepam, lorazepam, lormetazepam, meprobamate, midazolam, nitrazepam, oxazepam, prazepam, quazepam, reclazepam, tracazolate, trepipam, temazepam, triazolam, uldazepam, zolazepam and equivalents and pharmaceutically active isomer(s) and metabolite(s) thereof;

(v) anticonvulsants, such as, for example, carbamazepine, valproate, lamotrogine, gabapentin and equivalents and pharmaceutically active isomer(s) and metabolite(s) thereof;

(vi) Alzheimer's therapies, such as, for example, donepezil, memantine, tacrine and equivalents and pharmaceutically active isomer(s) and metabolite(s) thereof;

(vii) Parkinson's therapies, such as, for example, deprenyl, L-dopa, Requip, Mirapex, MAOB inhibitors such as selegine and rasagiline, comP inhibitors such as Tasmar, A-2 inhibitors, dopamine reuptake inhibitors, NMDA antagonists, Nicotine agonists, Dopamine agonists and inhibitors of neuronal nitric oxide synthase and equivalents and pharmaceutically active isomer(s) and metabolite(s) thereof;

(viii) migraine therapies, such as, for example, almotriptan, amantadine, bromocriptine, butalbital, cabergoline, dichloralphenazone, eletriptan, frovatriptan, lisuride, naratriptan, pergolide, pramipexole, rizatriptan, ropinirole, sumatriptan, zolmitriptan, zomitriptan, and equivalents and pharmaceutically active isomer(s) and metabolite(s) thereof;

(ix) stroke therapies, such as, for example, abciximab, activase, NXY-059, citicoline, crobenetine, desmoteplase, repinotan, traxoprodil and equivalents and pharmaceutically active isomer(s) and metabolite(s) thereof;

(x) urinary incontinence therapies, such as, for example, darafenacin, falvoxate, oxybutynin, propiverine, robalzotan, solifenacin, tolterodine and equivalents and pharmaceutically active isomer(s) and metabolite(s) thereof;

(xi) neuropathic pain therapies, such as, for example, gabapentin, lidoderm, pregablin and equivalents and pharmaceutically active isomer(s) and metabolite(s) thereof;

(xii) nociceptive pain therapies, such as, for example, celecoxib, etoricoxib, lumiracoxib, rofecoxib, valdecoxib, diclofenac, loxoprofen, naproxen, paracetamol and equivalents and pharmaceutically active isomer(s) and metabolite(s) thereof;

(xiii) insomnia therapies, such as, for example, agomelatine, allobarbital, alonimid, amobarbital, benzoctamine, butabarbital, capuride, chloral, cloperidone, clorethate, dexclamol, ethchlorvynol, etomidate, glutethimide, halazepam, hydroxyzine, mecloqualone, melatonin, mephobarbital, methaqualone, midaflur, nisobamate, pentobarbital, phenobarbital, propofol, ramelteon, roletamide, triclofos, secobarbital, zaleplon, zolpidem and equivalents and pharmaceutically active isomer(s) and metabolite(s) thereof;

(xiv) mood stabilizers including for example carbamazepine, divalproex, gabapentin, lamotrigine, lithium, olanzapine, quetiapine, valproate, valproic acid, verapamil, and equivalents and pharmaceutically active isomer(s) and metabolite(s) thereof;

(xv) obesity therapies, such as, for example, anti-obesity drugs that affect energy expenditure, glycolysis, gluconeogenesis, glucogenolysis, lipolysis, lipogenesis, fat absorption, fat storage, fat excretion, hunger and/or satiety and/or craving mechanisms, appetite/motivation, food intake, and G-I motility; very low calorie diets (VLCD); and low-calorie diets (LCD); and

(xvi) therapeutic agents useful in treating obesity associated disorders, such as, for example, biguanide drugs, insulin (synthetic insulin analogues) and oral antihyperglycemics (these are divided into prandial glucose regulators and alpha-glucosidase inhibitors), PPAR modulating agents, such as, for example, PPAR alpha and/or gamma agonists; sulfonylureas; cholesterol-lowering agents, such as, for example, inhibitors of HMG-CoA reductase (3-hydroxy-3-methylglutaryl coenzyme A reductase); an inhibitor of the ileal bile acid transport system (IBAT inhibitor); a bile acid binding resin; bile acid sequestering agent, such as, for example, colestipol, cholestyramine, or cholestagel; a CETP (cholesteryl ester transfer protein) inhibitor; a cholesterol absorption antagonist; a MTP (microsomal transfer protein) inhibitor; a nicotinic acid derivative, including slow release and combination products; a phytosterol compound; probucol; an anti-coagulant; an omega-3 fatty acid; an anti-obesity therapy, such as, for example, sibutramine, phentermine, orlistat, bupropion, ephedrine, and thyroxine; an antihypertensive, such as, for example, an angiotensin converting enzyme (ACE) inhibitor, an angiotensin II receptor antagonist, an adrenergic blocker, an alpha adrenergic blocker, a beta adrenergic blocker, a mixed alpha/beta adrenergic blocker, an adrenergic stimulant, calcium channel blocker, an AT-1 blocker, a saluretic, a diuretic, and a vasodilator; a melanin concentrating hormone (MCH) modulator; an NPY receptor modulator; an orexia receptor modulator; a phosphoinositide-dependent protein kinase (PDK) modulator; modulators of nuclear receptors, such as, for example, LXR, FXR, RXR, GR, ERRα, β, PPARα, β, γ and RORalpha; a monoamine transmission-modulating agent, such as, for example, a selective serotonin reuptake inhibitor (SSRI), a noradrenaline reuptake inhibitor (NARI), a noradrenaline-serotonin reuptake inhibitor (SNRI), a monoamine oxidase inhibitor (MAOI), a tricyclic antidepressive agent (TCA), a noradrenergic and specific serotonergic antidepressant (NaSSA); a serotonin receptor modulator; a leptin/leptin receptor modulator; a ghrelin/ghrelin receptor modulator; a DPP-IV inhibitor; and equivalents and pharmaceutically active isomer(s), metabolite(s), and pharamaceutically acceptable salts, solvates, and prodrugs thereof.

The above other pharmaceutically active compound, when employed in combination with the compounds of Formula (I), can be used, for example, in those amounts indicated in the Physicians' Desk Reference (PDR) or as otherwise determined by one of ordinary skill in the art.

Compounds in accordance with formula I can be administered by any means suitable for the condition to be treated, which can depend on the quantity of formula Ito be delivered.

Compound(s) in accordance with formula I may be administered in the form of a conventional pharmaceutical composition by any route including, but not limited to, for example, orally, intramuscularly, subcutaneously, topically, intranasally, intraperitoneally, intrathoracially, intravenously, epidurally, intrathecally, intracerebroventricularly, and injection into the joints.

In one embodiment, the route of administration is orally, intravenously or intramuscularly.

An “effective amount” of formula I may be determined by one of ordinary skill in the art, and includes exemplary dosage amounts for a mammal of from about 0.05 to about 300 mg/kg/day, preferably less than about 200 mg/kg/day, in a single dose or in or in the form of individual divided doses. Exemplary dosage amounts for an adult human are from about 1 to 100 (for example, 15) mg/kg of body weight of active compound per day, which can be administered in a single dose or in the form of individual divided doses, such as from 1 to 4 times per day.

The specific dose level and frequency of dosage for any particular subject, however, may vary and generally depends on a variety of factors, including, but not limited to, for example, the bioavailability of the specific formula I compound(s) in the administered form; metabolic stability and length of action of the specific formula I compound(s); species, age, body weight, general health, sex, and diet of the subject; mode and time of administration; rate of excretion; drug combination; and severity of the particular condition.

One embodiment provides a pharmaceutical composition comprising at least one compound in accordance with formula I and at least one pharmaceutically-acceptable carrier and/or diluent.

Another embodiment provides a method for treating at least one disorder selected from cognitive deficient in schizophrenia, narcolepsy, obesity, and alzheimer's disease in a warm-blooded animal, comprising administering to said animal in need of such treatment a pharmaceutical composition comprising a therapeutically effective amount of a compound according to formula I, and at least one pharmaceutically-acceptable carrier and/or diluent.

Acceptable solid pharmaceutical compositions include, but are not limited to, for example, powders, tablets, dispersible granules, capsules, cachets, and suppositories.

In a solid pharmaceutical composition, pharmaceutically acceptable carriers include, but are not limited to, for example, at least one solid, at least one liquid, and mixtures thereof. The solid carrier can also be a diluent, flavoring agent, solubilizer, lubricant, suspending agent, binder, encapsulating material, and/or table disintegrating agent. Suitable carriers, include, but are not limited to, for example, magnesium carbonate; magnesium stearate; talc; lactose; sugar; pectin; dextrin; starch; tragacanth; methyl cellulose; sodium carboxymethyl cellulose; a low-melting wax; cocoa butter; and mixtures thereof.

A powder can be prepared by, for example, mixing a finely divided solid with at least one finely divided compound of formula I.

A tablet can be prepared by, for example, mixing at least one formula I compound in suitable proportions with a pharmaceutically acceptable carrier having the necessary binding properties and compacted into the desired shape and size.

A suppository can be prepared by, for example, mixing at least one compound of formula I with at least one suitable non-irritating excipient that is liquid at rectal temperature but solid at a temperature below rectal temperature, wherein the non-irritating excipient is first melted and the formula I compound dispersed therein. The molten homogeneous mixture in then poured into convenient sized molds and allowed to cool and solidify. Exemplary non-irritating excipients include, but are not limited to, for example, cocoa butter; glycerinated gelatin; hydrogenated vegetable oils; mixtures of polyethylene glycols of various molecular weights; and fatty acid esters of polyethylene glycol.

Acceptable liquid pharmaceutical compositions include, but are not limited to, for example, solutions, suspensions, and emulsions. For example, sterile water or water propylene glycol solutions of at least one compound in accordance with formula I are liquid pharmaceutical compositions suitable for parenteral administration. Liquid compositions can also be formulated in solution in aqueous polyethylene glycol solution.

Aqueous solutions for oral administration can be prepared by dissolving at least one compound in accordance with formula I in water and adding suitable colorants, flavoring agents, stabilizers, and/or thickening agents as desired.

Aqueous suspensions for oral administration can be prepared by dispersing at least one finely divided compound of formula I in water together with a viscous material, such as, for example, a natural synthetic gum, resin, methyl cellulose, and sodium carboxymethyl cellulose.

In one embodiment, the pharmaceutical composition contains from about 0.05% to about 99% w (percent by weight) of at least one compound in accordance with formula I. All percentages by weight being based on total composition.

In another embodiment, the pharmaceutical composition contains from about 0.10% to about 50% w(percent by weight) of at least one compound in accordance with formula I. All percentages by weight being based on total composition.

Another embodiment, provides a pharmaceutical composition comprising a compound of formula I and a pharmaceutically acceptable carrier/diluent for therapy.

Yet a further embodiment provides a pharmaceutical composition comprising a compound of formula I and a pharmaceutically acceptable carrier/diluent for therapy

Further, there is provided a pharmaceutical composition comprising a compound of formula I, or a pharmaceutically acceptable salt thereof, in association with a pharmaceutically acceptable carrier use in any of the conditions discussed above.

In a further aspect, the present invention provides a method of preparing a compound of formula I.

### Biological Evaluation

At least one compound of formula I including the compounds described in the Examples hereof, when tested in at least one in vitro assay described below is active towards H3 receptors. Particularly, at least one compound of the invention is an effective H3 receptor ligand. In the in vitro assay, a compound can be tested for its activity toward H3 receptors and IC₅₀ obtained to determine the activity for a particular compound toward the H3 receptor.

Histamine H₃ SPA with the Agonist Radioligand [³H]—N-α-methylhistamine

The H3 binding assay was used to evaluate the ability of at least one compound in accordance with formula I to inhibit [³H]—N-α-methylhistamine binding to CHO-K1 membranes expressing human histamine H3 receptors (full-length H3, the most prevalent brain isoform 445). In 200 μl 96-well SPA format, human H3 membranes (12.5 μg protein/well) and 1.4 nM [³H]—N-α-methylhistamine were incubated with at least one compound in accordance with formula I for 1.5 hrs to determine percent effect with respect to total (1% DMSO) and non-specific binding (10 μM imetit). Reproducibility of the assay is such that IC₅₀ curves can be generated in singlicate. Single poke (SP) testing was done in triplicate.

Membranes, prepared from CHO-K1 cells stably expressing the human histamine H3 receptor, were obtained from ACS.

Tested formula I compounds were provided as solubilized samples in neat DMSO. Serial dilutions were performed in DMSO.

Plates were 96-well Unifilter GF/B (Perkin Elmer, 6005177). Plates were read on a Perkin Elmer TopCount. CPM data were used to analyze unless DPM data generated by a quench curve were required.

### A. Prep Work

1. 1 mg/ml BSA was added to assay buffer (AB) on day of assay.
2. Amounts required for bead/membrane pool in AB were calculated, “P”−needed 17.1 ml/assay plate+10 ml PlateMate excess. Buffer volume was split between beads and membranes to allow for polytroning of membranes prior to addition to beads.
   a. PVT-WGA SPA Beads: beads (P×9.83 mg/ml) were resuspended for 1750 μg/well final. Waited a minimum of 15 minutes prior to adding membranes (See b. below.).
   b. Membranes (hH3 membranes from CHO cells containing recombinant human H3 receptors, 11.7 mg/ml): membranes were removed from −80° C. and thawed in RT waterbath. (0.0702 mg/ml×P) mg of membranes were resuspended in the remaining volume that was not used with beads above for 12.5 μg/well final and homogenized briefly at polytron speed 5.0. The homogenized membrane mixture was combined with the beads and a minimum of 30 minutes was waited prior to dispensing to plate.
3. Formula I compounds: For Single Poke, 2 μl 1 mM of a compound in accordance with formula I was dispensed to Optiplates (triplicate plates) for final concentration of 10 μM. (CMA dispensed 2.2 μl of 0.909 mM.) For IC₅₀, 6 μl of a compound in accordance with formula I was placed in DMSO in column 1 of a 96-well 500 μl polypropylene U-bottom plate for top final concentration of 10 μM. Imetit (see below) was used as a control.
4. Imetit (for NSB and control): a 100 μM solution in DMSO was prepared for a final assay concentration of 1 μM (NSB) or 100 nM (IC₅₀).
5. [³H]—N-α-methylhistamine ([³H]-NAMH): A solution in AB at 14 nM, 10× final concentration of 1.4 nM was prepared. 5 μl samples were calculated in quadruplicate on the β counter. If concentration is 12-14.5 nM, no adjustment is required. (For IC₅₀s, use final concentration on calculation tab of ABase template.)

### B. Assay

1. For IC₅₀s: a compound in accordance with formula I was diluted 1:10 in DMSO (6 μl+54 μl DMSO were added by PlateMate), and 1:3 serial dilutions (30 μl+60 μl) were then prepared in DMSO for a top final dilution of 1:1000 from stock concentration.
2. 2 μl of the formula I compound dilution was mixed and then transferred into assay plates. DMSO was removed and 2 μl of 100 μM Imetit was added to the wells.
3. 178 μl bead/membrane mixture was dispensed into the assay plate.
4. 20 μl [³H]-NAMH was added with Rapid Plate. The assay plate was sealed and incubated for 1.5 hr on RT shaker at speed ˜6.5.
5. The assay plate was subsequently centrifuged at 1000 rpm for 10 minutes.
6. The count was performed on TopCount using one of the 3H SPA H3 Quench programs.

The DPM data was analyzed when tSIS was less than that associated with 70% of full scale on the quench curve (tSIS<25%). Otherwise, CPM data was used. A typical window was 800-1200 CPM total, 45-70 CPM NSB (Z′ 0.70-0.90).

The Data was analyzed by calculating percent effect {average of [1−(singlicate minus plate NSB)/(plate Total minus plate NSB)]×100%}, IC₅₀, and Ki using the Cheng-Prusoff equation below and an ActivityBase or XLfit template.

$$Ki = \frac{{IC}_{50}}{1 + ( [ ligand ] / Kd )}$$

where Kd is the value for the [³H] ligand (0.67 nM)

In this assay, the ligand was adjusted to 1.4 nM, which is ˜2× the average Kd (0.67 nM).

The IC₅₀ and nH were determined by fitting the data to model 205 in XLfit: y=A+((B−A)/(1+((C/x)̂D)).

Guanosine 5′-O-(3-[³⁵]thio)triphosphate [GTPγS] Binding Assay

The GTPγS binding assay can be used to investigate antagonist properties of compounds in CHO cells (Chinese Hamster Ovary) transfected with human Histamine H3 receptor (hH3R). Membranes from CHO cells expressing hH3R (10 μg/well) are diluted in GTPγS assay buffer (20 mM Hepes, 10 mM MgCl₂, 100 mM NaCl, pH 7.4) and preincubated with saponine (3 μg/ml), GDP (10 μM) and PVT-WGA SPA beads (125 μg/well) (Amersham) for 30 minutes. To determine antagonist activity, (R)-α-methyl histamine (30 nM) is added in 96 well SPA plate with [³⁵S]GTPγS (0.2 nM) and various concentration of H3R antagonists. The GTPγS binding assay is started with addition of the mixture membrane/saponine/GDP and incubated for 90 minutes at room temperature. The amount of bound [³⁵S]GTPγS is determined by using the MicroBeta Trilux counter (PerkinElmer). The percentage of [³⁵S]GTPγS bound in each sample is calculated as a percentage of that bound control sample incubated in absence of H3 antagonist. Duplicate determinations are obtained for each concentration, and the data are analyzed using ExcelFit4 to obtain the IC₅₀.

### IC₅₀ Values

At least one formula I compound in accordance with the present invention has an IC₅₀ value of less than about 100 μM. In a further embodiment, at least one compound of formula I showed activity in at least one of the above referenced assays via an IC₅₀ value of between about 1 nm to about 100 μM. In an even further embodiment, at least one compound of formula I showed activity in at least one of the above referenced assays via an IC₅₀ value of between about 2 nM to about 100 nM. In yet a further embodiment, at least one compound of formula I showed activity in at least one of the above referenced assays via an IC₅₀ value of between about 2 nM and 50 nM. In still a further embodiment, at least one compound of formula I showed activity in at least one of the above referenced assays via an IC₅₀ value of between about 1 nM and 1400 nM. In one embodiment, at least one compound of formula I showed activity in at least one of the above referenced assays via an IC₅₀ value of less than about 1400 nM. In another embodiment, at least one compound of formula I showed activity in at least one of the above referenced assays via an IC₅₀ value of less than about 100 nM. In yet another embodiment, at least one compound of formula I showed activity in at least one of the above referenced assays via an IC₅₀ value of less than about 50 nM. In yet another embodiment, at least one compound of formula I showed activity in at least one of the above referenced assays via an IC₅₀ value of less than about 10 nM.

Set forth in Table 1 hereinbelow for the Example 1-50 compounds are IC₅₀ values that were generated in accordance with the histamine H₃ SPA Assay as essentially described hereinabove and/or GTPγS Binding Assay as essentially described hereinabove.

TABLE 1

| | hH3 binding | GTPγS Binding |
|---|---|---|
| EX No. | IC₅₀ (nM) | IC₅₀ (nM) |
| 1 | 33.6 | 13 |
| 2 | 390 | Not tested |
| 3 | 1000 | Not tested |
| 4 | 800 | Not tested |
| 5 | 640 | Not tested |
| 6 | 1300 | Not tested |
| 7 | 1400 | 1200 |
| 8 | 4.1 | 3.66 |
| 9 | 33.5 | 19 |
| 10 | 9.02 | 3.25 |
| 11 | 196 | 74 |
| 12 | 4.43 | 8.8 |
| 13 | 573 | Not tested |
| 14 | 26.1 | 139 |
| 15 | 322 | Not tested |
| 16 | 65.2 | 16.5 |
| 17 | 27.4 | 16.4 |
| 18 | 407 | 125 |
| 19 | 56.5 | 10.1 |
| 20 | 97.2 | 22.5 |
| 21 | 141 | 19.3 |
| 22 | 209 | 67 |
| 23 | 306 | Not tested |
| 24 | 336 | Not tested |
| 25 | 419 | Not tested |
| 26 | 1.35 | 6.55 |
| 27 | 7.68 | 44 |
| 28 | 2.66 | 13.2 |
| 29 | 50.9 | 87.5 |
| 30 | 10.1 | 14.3 |
| 31 | 11.3 | 13.5 |
| 32 | 15 | 17 |
| 33 | 35.1 | 5.5 |
| 34 | 24.4 | 13.5 |
| 35 | 165 | 85.3 |
| 36 | 182 | Not tested |
| 37 | 207 | Not tested |
| 38 | 336 | Not tested |
| 39 | 361 | Not tested |
| 40 | 470 | Not tested |
| 41 | 542 | Not tested |
| 42 | 594 | Not tested |
| 43 | 615 | Not tested |
| 44 | 736 | Not tested |
| 45 | 791 | Not tested |
| 46 | 10,000 | Not tested |
| 47 | 5,170 | Not tested |
| 48 | Not tested | 308.44 |
| 49 | Not tested | 248.52 |
| 50 | Not tested | 115.31 |

### EXAMPLES

The invention is further defined in the following Examples. It should be understood that the Examples are given by way of illustration only. From the above discussion and the Examples, one skilled in the art can ascertain the essential characteristics of the invention, and without departing from the spirit and scope thereof, can make various changes and modifications to adapt the invention to various uses and conditions. As a result, the invention is not limited by the illustrative examples set forth hereinbelow, but rather defined by the claims appended hereto.

All temperatures are in degrees Celsius (° C.). Unless otherwise stated, operations were carried out at room or ambient temperature (18-25° C.).

Unless otherwise noted, commercial reagents used in preparing the example compounds were used as received without additional purification.

Unless otherwise noted, the solvents used in preparing the example compounds were commercial anhydrous grades and were used without further drying or purification.

The names of the compounds exemplified herein were generated using either the StructToName component of CambridgeSoft's ChemOffice suite version 9.0.7, or AutoNom 2000 within ISIS/Draw. AutoNom (Automatic Nomenclature) is a chemical-name-generating program that assigns systematic IUPAC (International Union of Pure and Applied Chemistry) chemical names to drawn structures at the press of a button.

The following abbreviations are employed herein: ACN: acetonitrile; aq.: aqueous; atm: atmospheric pressure; BOC: 1,1-dimethylethoxycarbonyl; n-BuLi: n-butyllithium; DCE: dichloroethane; DCM or CH₂Cl₂: dichloromethane; DIPEA: N,N-Diisopropylethylamine; DMF: N,N-dimethylformamide; DMSO: dimethyl sulfoxide; EtOH: ethanol; Et₂O: diethyl ether; EtOAc: ethyl acetate; Eq: equivalents; h: hour(s); HCl: hydrochloric acid; H₂O: water; H₂O₂: hydrogen peroxide; HPLC: high performance liquid chromatography; EDC.HCl: 1-(3-Dimethylaminopropyl)-3-ethylcarbodiimide hydrochloride; HATU 0-(7-azabenzotriazole-1-yl)-N,N,N′N′-tetramethyluronium hexafluorophosphate; HBTU: O-Benzotriazole-N,N,N′,N′-tetramethyl-uronium-hexafluoro-phosphate; HOBT: 1-hydroxybenzotriazole; HRMS: high resolution mass spectrometry; K₂CO₃: potassium carbonate; KMnO₄: potassium permanganate; LiI: Lithium iodide; LiOH: lithium hydroxide; MeOH: methanol; min: minutes; MgSO₄: magnesium sulfate; min: minutes; MS: mass spectrum; NaCl: sodium chloride; Na₂CO₃ sodium carbonate; NaHCO₃: sodium bicarbonate; Na₂SO₄: sodium sulfate; NaH: sodium hydride; NaOH: sodium hydroxide; NH₃: ammonia; NH₄Cl: ammonium chloride; NH₄HCO₃: ammonium bicarbonate; NMR: nuclear magnetic resonance; Pd/C: palladium on carbon; psi: pounds per square inch; RT: room temperature; sat.: saturated; SiO₂: Silica gel; SOCl₂: thionyl chloride; THAI: tetrabutylammonium iodide; TEA: triethylamine; TFA: trifluoroacetic acid; and THF: tetrahydrofuran.

Chromatography means flash column chromatography on silica gel unless otherwise noted. Solvent mixture compositions are given as volume percentages or volume ratios.

### Normal Phase ISCO Chromatography Conditions:

Flash chromatography was employed as a method of purifying selected compounds and intermediates. Such purification was accomplished via an ISCO CombiFlash Sq 16× or ISCO Companion instrument using pre-packaged disposable RediSep SiO₂ stationary phase columns (4, 12, 40, 120 and 330 gram sizes) with gradient elution at 5-100 mL/min of selected bi-solvent mixture, UV detection (190-760 nm range) or timed collection, 0.1 mm flow cell path length.

### Reverse Phase High pH Chromatography Conditions:

Compounds were purified on preparative HPLC MS (reverse phase) using the long high pH 20 to 40% (ACN in water ammonium carbonate buffer, 25 min) gradient method on XBridge Prep C18 OBD, 30×150 mm, 5 mm, Waters reverse phase column.

### LC-MS HPLC Conditions:

Method A. LC-MS HPLC was determined for the Example 1-14, 16-37, 43, 44, 46, and 47 compounds in accordance with Method A. Agilent Zorbax SB-C column 5 □m, 2.1 mm ID×50 mm, 1.4 mL/min flow rate, and a gradient of 95% A to 90% B over 3 min hold 1 min ramp down to 95% A over 1 min and hold 1 min A=2% ACN in water with 0.1% formic acid and B=2% water in ACN with 0.1% formic acid. UV-DAD was conducated at 210-400 nm. The MS detection was performed with a Micromass Platform ZMD or LCZ spectrometers using the indicated ionization method. Retention time (t_{R})=min High-resolution mass spectra were recorded on an Agilent Technologies 6210 Time-of-Flight LC/MS spectrometer.

Method B. LC-MS HPLC was determined for the Example 15, 38-42, and 45 compounds in accordance with Method B. LC/MS Conditions, (long run): LC/MS data was obtained on an Agilent Technologies 1100 LC platform with UV-diode array detection, 214 nm, and base peak intensity (BPI) reporting. LC conditions: Gemini C18, 100×4.6 mm column; 1.2 mL/min; gradient: 95% A to 10% B over 2 mins, ramp up to 93% B over 16 mins, ramp back down to 95% A over 1 min and hold for 1 min. Where A=0.125 M Ammonium Bicarbonate in water and B=100% Acetonitrile. The column temperature was maintained at 30° C. The MS detection was performed with a Micromass LCT spectrometer using the indicated ionization method/detection method. Retention time (t_{R})=min

Method C. LC-MS HPLC was determined for the Example 48-50 compounds in accordance with Method C. Agilent 1100 Zorbax SB C-18 column 1.8 □m, 4.6 mm ID×30 mm, 3.5 mL/min flow rate, and a gradient of 5% to 95% B over 4.5 min A=0.05% TFA in water and B=0.05% TFA in ACN. UV-DAD was conducted at 210-400 nm. The MS detection was performed with a Agilent 1100 Series LC/MSD spectrometer using APPI as ionization method. Retention time (t_{R})=min High-resolution mass spectra were recorded on an Agilent LC/MSD TOF spectrometer.

### NMR Conditions:

Method A. NMR was determined for the Example 1-47 compounds in accordance with Method A. Proton magnetic resonance (¹H NMR) and (¹³C NMR) spectra were recorded on a Bruker Avance DPX 300 mHz, 400 mHz, or 500 mHz spectrometer and the chemical shifts are reported in parts-per-million (6) from a tetramethylsilane internal standard.

Method B. NMR was determined for the Example 48-50 compounds in accordance with Method B. Proton magnetic resonance (¹H NMR) spectra were recorded on a Varian Unity Plus 400 MHz spectrometer and the chemical shifts were recorded in parts per million from a tetramethylsilane internal standard.

### Example 1

### (4-Cyclohexyl-piperazin-1-yl)-[6-(tetrahydro-pyran-4-yl)-6-aza-spiro[2.5]oct-1-yl]-methanone

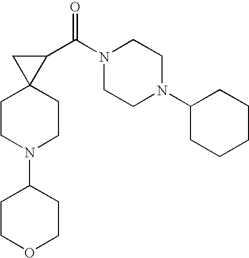

### 1A. 4-methylene-piperidine-1-carboxylic acid benzyl ester

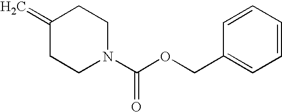

A solution of methyltriphenylphosphonium bromide (88.2 mmol, 31.52 g) was cooled in 600 mL of THF to 0° C. To this solution, n-BuLi (2.5M, in hexanes, 35.3 mL, 1.2 eq.) was slowly added; a precipitate gradually formed and the reaction turned reddish-orange in color. The reaction was stirred for 1 h at 0° C. at which point 4-Oxo-piperidine-1-carboxylic acid benzyl ester, which is commercially available from Aldrich, was added (73.5 mmol, 17.15 g, in 30 mL THF) and the reaction was stirred for an additional 1 h at 0° C. The volatiles were removed under reduced pressure and the reaction mixture was poured on to a pad of SiO₂ and flushed with a 4:1 mixture of hexanes/EtOAc. The product was purified via column chromatography with 1A eluting with 15% EtOAc and being isolated as a clear oil (54%). m/z ((AP+) Direct infusion) (M+H)⁺=232.1. ¹H NMR (300.132 MHz, CDCl₃) □2.23 (t, J=5.7 Hz, 4H), 3.53 (t, J=5.9 Hz, 4H), 4.78 (s, 2H), 5.17 (s, 2H), 7.30-7.39 (m, 5H).

### 1B. 6-Aza-spiro[2.5]octane-1,6-dicarboxylic acid 6-benzyl ester 1-ethyl ester

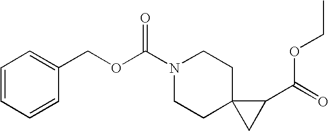

Method 1: 1A (39.6 mmol, 9.17 g) was dissolved in 1300 ml of anhydrous DCM followed by the addition of CuCN (39.6 mmol 3 54 g) and the reaction was stirred at RT. Ethyl diazoacetate (87.22 mmol, 9.17 ml) was dissolved in 10 ml of DCM and slowly added (0.2 mL/hr, over roughly 100 h). The solution was filtered through SiO₂ and the volatiles removed. The residue was purified via column chromatography (100% hexanes to 4:1 hexanes/EtOAc) to give 5.68 g 1B as a clear oil (45%). m/z (AP+) M+1=318.2; HPLC t_{R}=2.69 min ¹H NMR (300.132 MHz, CDCl₃) δ 0.95 (dd, J=8.0, 4.6 Hz, 1H), 1.20 (t, J=5.0 Hz, 1H), 1.28 (t, J=7.1 Hz, 3H), 1.42-1.48 (m, 2H), 1.56-1.61 (m, 1H), 1.69-1.76 (m, 2H), 3.33-3.41 (m, 1H), 3.49-3.65 (m, 3H), 4.16 (q, J=8.4 Hz, 2H), 5.16 (s, 2H), 7.35-7.38 (m, 5H).

Method 2: To a solution of 1A (13 g, 56 2 mmol) in xylene (5 ml) was added copper powder (100 mg) and then heated to 85-90° C. A solution of ethyl diazoacetate (17.7 ml, 168 3 mmol) in xylene (5 ml) was added drop wise in 1 h and further maintained at same temperature for 20 min, in cases where any starting material remains more ethyl diazoacetate was added. The reaction was cooled to RT and solvent was removed under reduced pressure. The crude material was purified by column chromatography over silica gel using 0-16% EtOAc in petroleum ether as an eluent to give 6.59 g 1B as a thick liquid (37%). ¹H NMR (400 MHz, CDCl₃): δ 0.89-0.96 (m, 2H), 1.18 (t, 1H, J=4.96 Hz), 1.25-1.32 (t, 3H), 1.40 (m, 2H), 1.56-1.59 (m, 1H), 1.72 (m, 1H), 3.33-3.39 (m, 1H), 3.52-3.59 (m, 3H), 4.11-4.17 (q, 2H), 5.14 (s, 2H), 7.31-7.36 (m, 5H).

### 1C. 6-Aza-spiro[2.5]octane-1,6-dicarboxylic acid 6-benzyl ester

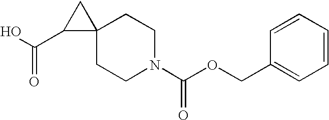

1B (4.06 mmol, 1.29 g) was dissolved in MeOH (8 mL), THF (8 mL) and H₂O (8 mL) and to this was added LiOH (8.13 mmol, 0.33 g) and the reaction was stirred overnight at RT. After the reaction was complete, it was acidified to pH=1 with 1N HCl and extracted with EtOAc. The organics were dried over MgSO₄, filtered and the residual material dried under vacuum to give 1.1 g 1C as a white solid (94%). m/z (AP+) M+1=290.1; HPLC t_{R}=2.19 min ¹H NMR (300.132 MHz, CDCl₃) δ 0.83-0.94 (m, 1H), 1.09-1.15 (m, 1H), 1.30-1.40 (m, 2H), 1.46-1.52 (m, 1H), 1.61-1.70 (m, 2H), 3.23-3.54 (m, 4H), 5.06 (s, 2H), 7.20-7.28 (m, 5H).

### 1D. 1-(4-tertButoxycarbonyl-piperazine-1-carbonyl)-6-aza-spiro[2.5]octane-6-carboxylic acid benzyl ester

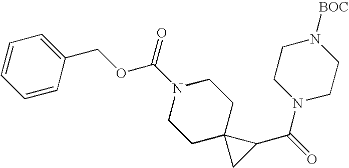

Method 1: 1C (1.90 mmol, 0.55 g), 0-(7-azabenzotriazole-1-yl)-N,N,N′N′-tetramethyluronium hexafluorophosphate (3.80 mmol, 1.44 g), N-ethyldiisopropylamine (3.80 mmol, 0.66 mL) and piperazine-1-carboxylic acid tert-butyl ester (2.85 mmol, 0.53 g), which are all commercially available from Aldrich, were combined in 50 mL of anhydrous ACN and stirred at RT overnight. The reaction was diluted with EtOAc and washed with 100 mL of 1 N NaOH. The organics were then dried over MgSO₄, filtered through a silica gel plug and the volatiles removed under reduced pressure. The residue was purified by column chromatography and 1D isolated via elution with a hexanes/EtOAc gradient (100% hexanes to 50% hexanes over 30 min) to obtain 0.60 g 1D as a light tan solid (69%). m/z (AP+) M+Na=480.2; HPLC t_{R}=2.63 min ¹H NMR (300.132 MHz, CDCl₃) δ 0.74 (dd, J=7.7, 4.6 Hz, 1H), 1.19-1.27 (m, 2H), 1.40 (s, 9H), 1.45-1.62 (m, 4H), 3.18-3.69 (m, 12H), 5.06 (s, 2H), 7.34 (s, 5H).

Method 2: To a solution of 1C (6.3 g, 21.1 mmol) in dry CH₂Cl₂ (30 ml) was added N—Boc-piperazine (6.08 g, 32.6 mmol), EDCI-HCl (6.67 g, 32 6 mmol), HOBT (50 mg) and TEA (6.61 g, 65.3 mmol). The reaction mixture was stirred at RT over night, and then diluted with CH₂Cl₂, washed with water, dried over Na₂SO₄ and concentrated under vacuum. The crude material was purified by column chromatography over silica gel using 0-50% of EtOAc in petroleum ether as an eluent to give 7.0 g 1D as an off white solid (70%). ¹H NMR (400 MHz, CDCl₃): δ 0.86-0.90 (m, 2H), 1.33-1.34 (m, 3H), 1.4-1.8 (m, 3H), 1.48 (s, 9H), 3.27-3.74 (m, 12H), 5.14 (s, 2H), 7.30-7.37 (m, 5H).

### 1E. 4-(6-Aza-spiro[2.5]octane-1-carbonyl)-piperazine-1-carboxylic acid tert-butyl ester

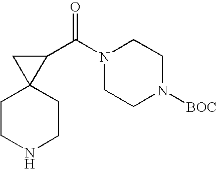

1D (1.31 mmol, 0.60 g) was dissolved in 100 mL of EtOH and purged with N₂ for 15 min. This was followed by the addition of 3 drops of HOAc and 0.06 g of 5% Pd/C (10% by weight of piperazine) and the reaction was placed under a balloon of H₂; the reaction was then stirred overnight at RT. The reaction was filtered through a celite plug and the volatiles removed under reduced pressure. The residue was purified via column chromatography and eluted with a DCM/DCM containing 10% (2 M NH₃/MeOH) gradient over 30 min to give 0.42 g 1E as a clear oil (99%). m/z (AP+) M+1=324.3; HPLC t_{R}=1.45 min ¹H NMR (300.132 MHz, CD₃OD) δ 0.79 (dd, J=7.8, 4.3 Hz, 1H), 1.15 (t, J=4.8 Hz, 1H), 1.32-1.44 (m, 2H), 1.47 (s, 9H), 1.50-1.68 (m, 2H), 1.83 (dd, J=7.8, 5.3 Hz, 1H), 2.72-2.76 (m, 2H), 2.83-2.89 (m, 2H), 3.37-3.78 (m, 8H).

### 1F. 4-[6-(Tetrahydro-pyran-4-yl)-6-aza-spiro[2.5]octane-1-carbonyl]-piperazine-1-carboxylic acid tert-butyl ester

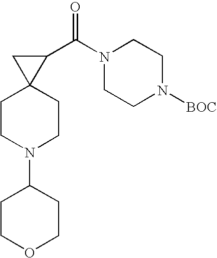

1E (1.30 mmol, 0.42 g), tetrahydro-pyran-4-one (1.95 mmol, 0.13 mL), 3 drops of HOAc, and NaBH₃CN (2.92 mmol, 0.183 g) were combined in 60 mL of anhydrous EtOH and stirred 12 h at RT followed by heating to reflux for 6 h. The reaction was cooled to RT, diluted with EtOAc and washed with 50 mL of 1N NaOH. The organics were dried over Na₂SO₄, filtered through a glass filter frit and the volatiles removed under reduced pressure. The residue was purified by column chromatography, which was eluted with a DCM/DCM containing 10% (2M NH₃/MeOH) gradient over 30 min to produce 0.44 g 1F as a clear oil (83%). m/z (AP+) M+1=408.3; HPLC t_{R}=1.54 min ¹H NMR (300.132 MHz, CDClₐ) δ 4.03 (dd, J=11.4, 3.5 Hz, 2H), 3.58-3.34 (m, 6H), 2.97-2.72 (m, 5H), 1.97-1.80 (m, 2H), 1.75-1.55 (m, 4H), 1.55-1.39 (m, 4H), 1.44 (s, 9H), 1.23 (t, J=4.8 Hz, 2H), 0.82 (dd, J=8.0, 4.6 Hz, 2H).

### 1G. piperazin-1-yl-[6-(tetrahydro-pyran-4-yl)-6-aza-spiro[2.5]oct-1-yl]-methanone Bis trifluoromethylacetic acid salt

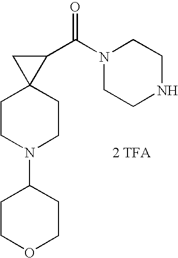

1F (1.08 mmol, 0.44 g) was dissolved in 25 mL of DCM followed by the addition of 5 mL of TFA and the reaction was stirred at RT for 1.5 h. The volatiles were removed under reduced pressure and the product dried overnight under high vacuum to give 0.57 g 1G as a tan TFA salt (99%), which was used without additional purification. m/z (AP+) M+1=308.3; HPLC t_{R}=0.21 min ¹H NMR (300.132 MHz, CD₃OD) δ 4.13-3.71 (m, 4H), 3.69-3.55 (m, 6H), 3.49-3.06 (m, 5H), 2.15-1.95 (m, 4H), 1.89-1.71 (m, 2H), 1.65-1.24 (m, 4H), 1.11-0.97 (m, 2H).

### 1H. (4-Cyclohexyl-piperazin-1-yl)-[6-(tetrahydro-pyran-4-yl)-6-aza-spiro[2.5]oct-1-yl]-methanone

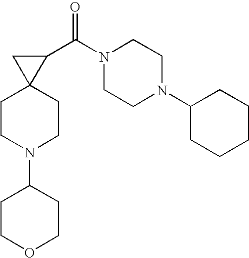

1G (0.36 mmol, 0.193 g), cyclohexanone (1.079 mmol, 0.112 mL), 3 drops of HOAc, and polystyrylmethyl trimethylammonium cyanoborohydride (0.70 mmol, 0.19 g, 4.1 mmol/g of resin) were dissolved in 20 ml of DCM and stirred at RT overnight. The solution was filtered through a nylon filter and the volatiles removed. The residue was purified via column chromatography and the product eluted with a DCM/DCM contain 10% (2 M NH₃/MeOH) gradient over 30 min 92.5 mg 1H was isolated as a white solid (66%). m/z (AP+) M+1=390.0; HPLC t_{R}=0.32 min ¹H NMR (300.132 MHz, DMSO-D₆) δ 0.61-0.65 (m, 1H), 0.95-0.98 (m, 1H), 1.07-1.22 (m, 6H), 1.30-1.55 (m, 6H), 1.55-1.82 (m, 6H), 2.25-2.58 (m, 11H), 3.22-3.29 (m, 2H), 3.58-3.64 (m, 3H), 3.84-3.89 (m, 2H).

### Example 2

### (4-Cyclohexylmethyl-piperazin-1-yl)-[6-(tetrahydro-pyran-4-yl)-6-aza-spiro[2.5]oct-1-yl]-methanone

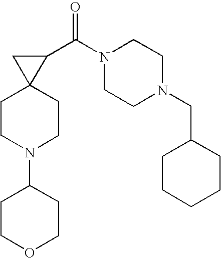

1G (0.36 mmol, 0.193 g), cyclohexanecarbaldehyde (1.07 mmol, 0.121 g), which is available from Aldrich, 3 drops of HOAc, and polystyrylmethyl trimethylammonium cyanoborohydride (0.70 mmol, 0.19 g, 4.1 mmol/g of resin), which is available from Novabiochem were dissolved in 10 ml of THF and stirred at RT overnight. The solution was filtered through a nylon filter and the volatiles removed. The residue was purified by column chromatography and was eluted with a DCM/DCM containing 10% (2M NH₃/MeOH) gradient 30 min 38 mg of title product was isolated as a oil (26%) and was converted to the citrate salt by adding 18.1 mg (0.941 mmol, 1 eq.) citric acid in MeOH and removing the solvent under reduced pressure. m/z (AP+) M+1=404.3; HPLC t_{R}=0.83 min Freebase: ¹H NMR (300.132 MHz, CD₃OD) δ 0.75-0.79 (m, 1H), 0.79-0.97 (m, 2H), 1.12-1.35 (m, 4H), 1.47-1.82 (m, 15H), 2.17 (d, J=7.0 Hz, 2H), 2.29-2.73 (m, 8H), 3.29-3.52 (m, 4H), 3.70-3.72 (m, 3H), 3.96-4.00 (m, 2H).

### Example 3

### [6-(2-Ethyl-butyl)-6-aza-spiro[2.5]oct-1-yl]-(4-methyl-piperazin-1-yl)-methanone

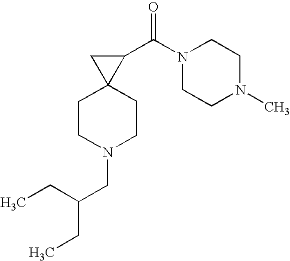

### 3A. (4-Methyl-piperazine-1-carbonyl)-6-aza-spiro[2.5]octane-6-carboxylic acid benzyl ester

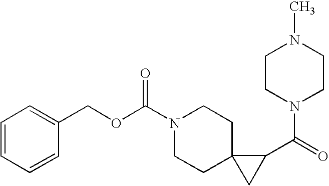

1C (3.80 mmol, 1.10 g), HATU (5.70 mmol, 2.16 g), N-ethyldiisopropylamine (11.40 mmol, 1.99 mL), and 1-methyl-piperazine (7.60 mmol, 0.76 g), which are all available from Aldrich, were combined in 50 mL of anhydrous ACN and stirred at RT overnight. The reaction was diluted with EtOAc and washed with 100 mL of H₂O sat. with Na₂CO₃. The organics were dried over Na₂SO₄, filtered through a flitted glass filter and the volatiles removed under reduced pressure. The residue was purified by column chromatography and the product eluted with a DCM/DCM containing 10% (2M NH₃/MeOH) gradient over 30 min 1.40 g 3A was isolated as a tan solid (99%). m/z (AP+) M+1=372.3; HPLC t_{R}=1.69 min ¹H NMR (300.132 MHz, CDCl₃) δ 0.78 (dd, J=7.9, 4.5 Hz, 1H), 1.24-1.46 (m, 4H), 1.54-1.66 (m, 4H), 2.32 (s, 3H), 2.37-2.53 (m, 2H), 3.26-3.42 (m, 2H), 3.53-3.76 (m, 6H), 5.13 (s, 2H), 7.30-7.36 (m, 5H)

### 3B. (6-Aza-spiro[2.5]oct-1-yl)-(4-methyl-piperazin-1-yl)-methasone.

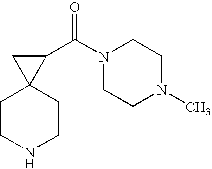

3A (3.77 mmol, 1.40 g) was dissolved in 100 mL of EtOH and degassed with N₂ for 15 min 0.14 g of 5% Pd/C and 3 drops of HOAc were then added and the reaction vessel placed under a balloon of H₂ and stirred at RT, the reaction required 3 days to go to completion. The reaction was filtered through a pad of celite and the volatiles removed under reduced pressure. The residue was purified via column chromatography and the product eluted with DCM/DCM containing 10% (2M NH₃/MeOH) gradient over 30 min 1.28 g of freebase 3B was isolated as a clear oil (96%), which slowly discolored over the course of 3 weeks, necessitating an additional purification by repeating the above procedure. m/z (AP+) M+1=238.3; HPLC t_{R}=0.15 min ¹H NMR (300.132 MHz, CDCl₃) δ 0.70-0.77 (m, 3H), 1.22-1.43 (m, 4H), 1.55-1.70 (m, 2H), 1.70-1.86 (m, 2H), 2.25-2.54 (m, 3H), 2.25 (s, 3H), 2.83-3.09 (m, 4H), 3.40-3.50 (m, 3H), 5.34 (bs, 1H).

### 3C. [6-(2-Ethyl-butyl)-6-aza-spiro[2.5]oct-1-yl]-(4-methyl-piperazin-1-yl)-methanone

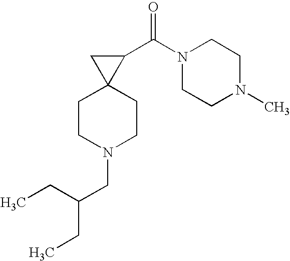

3B (0.463 mmol, 0.110 g), 2-Ethyl-butyraldehyde (0.927 mmol, 0.106 mL), 3 drops of HOAc, and polystyrylmethyl trimethylammonium cyanoborohydride (1.15 mmol, 0.28 g, 4.1 mmol/g of resin) were combined in 10 mL of anhydrous THF and the reaction heated to 65° C. overnight. The reaction was filtered through a nylon filter and volatiles removed under reduced pressure. The residue was purified by chromatography and the product eluted with DCM/DCM containing 10% (2M NH₃/MeOH) gradient over 30 min 0.0987 g of the freebase 3C (66%) was isolated as an oil and then converted to a solid white salt via adding citric acid (0.307 mmol, 0.059 g, 1 eq.) in 5 ml MeOH follow by removal of the solvent under reduced pressure. m/z (AP+) M+1=322.4; HPLC t_{R}=0.30 min Freebase: ¹H NMR (300.132 MHz, CDCl₃) δ 0.69-0.77 (m, 1H), 0.88 (t, J=0.3 Hz, 6H), 1.22-1.49 (m, 6H), 1.49-1.72 (m, 4H), 2.12-2.21 (m, 2H), 2.34 (s, 3H), 2.38-2.58 (m, 7H), 2.72-2.92 (m, 2H), 3.46-3.61 (m, 1H), 3.62-4.09 (m, 3H).

### Example 4

### (4-Methyl-piperazin-1-yl)-[6-((E)-3-phenyl-allyl)-6-aza-spiro[2.5]oct-1-yl]-methanone

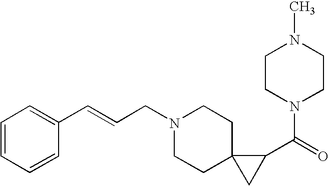

3B (0.417 mmol, 0.099 g), trans-cinnamaldehyde (0.83 mmol, 0.11 mL), 3 drops of HOAc, and polystyrylmethyl trimethylammonium cyanoborohydride (0.92 mmol, 0.22 g, 4.1 mmol/g of resin) were combined in 10 mL of anhydrous THF and the reaction heated to 65° C. overnight. The reaction was filtered through a nylon filter and volatiles removed under reduced pressure. The residue was purified by chromatography and the product eluted with DCM/DCM containing 10% (2M NH₃/MeOH) gradient over 30 min 0.0383 g title product as a freebase was isolated as an oil (26%) and then converted to a solid citrate salt via adding citric acid (0.108 mmol, 0.0208 g, 1 eq.) in 5 ml MeOH followed by removal of volatiles under reduced pressure. m/z (AP+) M+1=354.3; HPLC t_{R}=0.79 min Freebase: ¹H NMR (300.132 MHz, CDCl₃) δ 0.75 (dd, J=7.8, 4.4 Hz, 1H), 1.27 (t, J=4.7 Hz, 1H), 1.32-1.86 (m, 5H), 2.32 (s, 3H), 2.36-2.78 (m, 7H), 3.21 (d, J=6.6 Hz, 2H), 3.44-3.89 (m, 5H), 6.32 (dt, J=15.8, 6.8 Hz, 1H), 6.54 (d, J=15.8 Hz, 1H), 7.19-7.30 (m, 3H), 7.34 (d, J=7.0 Hz, 1H), 7.39 (d, J=7.1 Hz, 1H).

### Example 5

### (4-Methyl-piperazin-1-yl)-(6-phenethyl-6-aza-spiro[2.5]oct-1-yl)-methanone

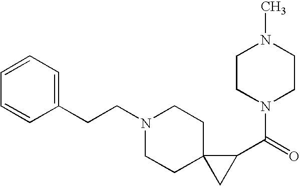

3B (0.417 mmol, 0.099 g), phenacetylaldehyde (0.83 mmol, 0.98 mL), 3 drops of HOAc, and polystyrylmethyl trimethylammonium cyanoborohydride (0.92 mmol, 0.22 g, 4.1 mmol/g of resin) were combined in 10 mL of anhydrous THF and the reaction heated to 65° C. overnight. The reaction was filtered through a nylon filter and the volatiles removed under reduced pressure. The residue was purified on a 40 g SiO₂ ISCO cartridge that was eluted with a DCM/DCM-10% (2M NH₃/MeOH) gradient (100% DCM to 0% DCM over 30 min) 0.0231 g of title product as a freebase was isolated as oil (16%) and then converted to a solid citrate salt via adding citric acid (0.0676 mmol, 0.0130 g, 1 eq.) in 5 ml MeOH follow by removal of the solvent under reduced pressure. m/z (AP+) M+1=342.2; HPLC t_{R}=0.37 min Freebase: ¹H NMR (300.132 MHz, CDCl₃) δ 0.76 (dd, J=7.8, 4.4 Hz, 1H), 1.28 (t, J=4.8 Hz, 1H), 1.40-1.55 (m, 3H), 1.62 (dd, J=7.8, 5.3 Hz, 1H), 1.67-1.85 (m, 2H), 2.33 (s, 3H), 2.36-2.56 (m, 5H), 2.62-2.72 (m, 4H), 2.82-2.88 (m, 2H), 3.47-3.59 (m, 1H), 3.63-3.72 (m, 2H), 3.75-3.87 (m, 1H), 7.22 (d, J=6.9 Hz, 2H), 7.30 (t, J=7.3 Hz, 3H).

### Example 6

### (4-Methyl-piperazin-1-yl)-[6-(1-methyl-piperidin-4-yl)-6-aza-spiro[2.5]oct-1-yl]-methanone

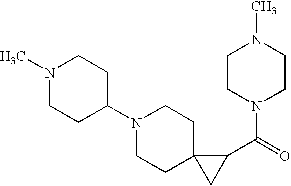

3B (0.417 mmol, 0.099 g), 1-methyl-piperidin-4-one (0.83 mmol, 0.96 mL), which is available from Aldrich, 3 drops of HOAc, and polystyrylmethyl trimethylammonium cyanoborohydride (0.917 mmol, 0.22 g, 4.1 mmol/g of resin) were combined in 10 mL of anhydrous THF and the reaction heated to 60° C. overnight. The reaction was filtered through a nylon filter and the volatiles removed under reduced pressure. The residue was purified via column chromatography and the product eluted with DCM/DCM containing 10% (2M NH₃/MeOH) gradient over 30 min 0.0597 g of title product as a freebase was isolated as an oil (43%) and then converted to a solid citrate salt via adding citric acid (0.178 mmol, 0.0343 g, 1 eq.) in 5 ml MeOH follow by removal of the solvent under reduced pressure. m/z (AP+) Direct infusion) (M+H)⁺=335.3. Freebase: ¹H NMR (300.132 MHz, CDCl₃) δ 0.72 (t, J=6.1 Hz, 1H), 0.72 (dd, J=7.8, 4.5 Hz, 1H), 1.32-1.50 (m, 2H), 1.55-1.85 (m, 7H), 1.96-2.07 (m, 2H), 2.26-2.65 (m, 12H), 2.69-2.75 (m, 1H), 2.80-2.86 (m, 2H), 2.97 (d, J=11.7 Hz, 2H), 3.51-3.55 (m, 1H), 3.60-3.67 (m, 2H), 3.72-3.84 (m, 1H).

### Example 7

### (4-Methyl-piperazin-1-yl)-[6-(tetrahydro-pyran-4-yl)-6-aza-spiro[2.5]oct-1-yl]-methasone

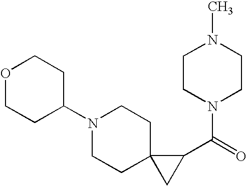

3B (0.417 mmol, 0.099 g), tetrahydro-pyran-4-one (0.83 mmol, 0.06 mL), which is available from Aldrich, 3 drops of HOAc, and polystyrylmethyl trimethylammonium cyanoborohydride (0.92 mmol, 0.22 g, 4.1 mmol/g of resin) were combined in 10 mL of anhydrous THF and the reaction heated to 60° C. overnight. The reaction was filtered through a nylon filter and the volatiles removed under reduced pressure. The residue was purified via column chromatography and the product eluted with DCM/DCM containing 10% (2M NH₃/MeOH) gradient over 30 min 0.0643 g of title compound as a freebase was isolated as an oil (48% yield) and then converted to a solid citrate salt via adding citric acid (0.20 mmol, 0.0384 g, 1 eq.) in 5 ml MeOH followed by removal of the solvent under reduced pressure. m/z (AP+) M+1=322.2; HPLC t_{R}=0.19 min Freebase: ¹H NMR (300.132 MHz, CDCl₃) δ 0.71-0.75 (m, 1H), 1.26 (t, J=4.7 Hz, 1H), 1.32-1.51 (m, 2H), 1.55-1.82 (m, 8H), 2.28 (s, 3H), 2.38-2.66 (m, 6H), 2.67-2.78 (m, 1H), 3.39 (td, J=11.7, 1.8 Hz, 2H), 3.46-3.60 (m, 2H), 3.66 (t, J=4.7 Hz, 2H), 3.72-3.84 (m, 1H), 4.04 (dd, J=11.1, 4.0 Hz, 2H).

### Example 8

### (4-Cyclobutylpiperazin-1-yl)(6-(tetrahydro-2H-pyran-4-yl)-6-azaspiro[2.5]octan-1-yl)methanone

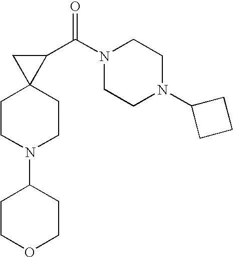

### 8A. Ethyl 6-azaspiro[2.5]octane-1-carboxylate

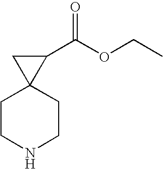

To semi-pure 1C (6.45 g, 20.32 mmol) in EtOH (100 mL) was added 5% Pd/C (0.649 g, 0.30 mmol) and a balloon filled with hydrogen placed on top of the reaction. The reaction was left to stir at RT over night. The mixture was filtered through Celite and the solvent removed under reduced pressure. The resulting oil was placed under high vacuum. The crude material was chromatographed on 100 g silica eluting first with 5% (7N NH₃/MeOH)/DCM then 10% 7N NH₃/MeOH)/DCM. The combined fractions were removed of solvent under reduced pressure to give 1.83 g 8A as an amber oil (44%). m/z (ES+) M+1=184; HPLC t_{R}=0.37 min; ¹H NMR (300.132 MHz, CDCl₃) δ 0.87 (dd, J=8.0, 4.4 Hz, 1H), 1.13 (t, J=4.9 Hz, 1H), 1.27 (t, J=7.1 Hz, 3H), 1.41 (q, J=5.2 Hz, 2H), 1.48-1.52 (m, 2H), 1.66-1.71 (m, 2H), 2.70-2.78 (m, 1H), 2.81-2.85 (m, 1H), 2.89 (t, J=5.4 Hz, 2H), 4.14 (q, J=7.1 Hz, 2H).

### 8B. ethyl 6-(tetrahydro-2H-pyran-4-yl)-6-azaspiro[2.5]octane-1-carboxylate

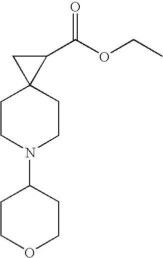

To 8A (1.847 g, 10.08 mmol) in DCE (25 mL) was added tetrahydro-4H-pyran-4-one (1.028 mL, 11.09 mmol) and the solution stirred 25 min. To this was added sodium triacetoxyborohydride (2.56 g, 12.10 mmol) and the reaction left to stir at RT. After 5 h the reaction was partitioned between EtOAc/sat. NaHCO₃. A trial batch of this reaction on 100 mg 8A was added to this aq. workup and the organic layers washed 3×sat. NaHCO₃, 1×sat. NaCl, dried over Na₂SO₄, solvent was removed under reduced pressure, and material put under high vacuum. The material was chromatographed on 50 g silica with 5% (7N NH₃/MeOH)/DCM and the combined purified fractions were removed of solvent to give 1.82 g 8B as a pale oil (64%). m/z (ES+) M+1=268; HPLC t_{R}=0.29 min, ¹H NMR (300.132 MHz, CDCl₃) δ 0.87 (dd, J=8.0, 4.5 Hz, 1H), 1.12 (t, J=4.9 Hz, 1H), 1.26 (t, J=7.1 Hz, 3H), 1.47-1.52 (m, 3H), 1.56-1.65 (m, 2H), 1.72-1.78 (m, 4H), 2.39-2.61 (m, 5H), 3.37 (td, J=11.7, 2.1 Hz, 2H), 4.02 (dd, J=11.5, 4.5 Hz, 2H), 4.13 (q, J=7.0 Hz, 2H).

### 8C. 6-(Tetrahydro-2H-pyran-4-yl)-6-azaspiro[2.5]octane-1-carboxylic acid

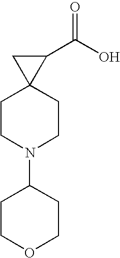

To 8B (1.820 g, 6.81 mmol) was added MeOH (25 mL), LiOH 1M (7.2 mL, 7.2 mmol), and H₂O (1 mL). The cloudy reaction was left to stir at RT. After 5 h 1N NaOH (7.2 mL, 7.2 mmol) and 20 mL MeOH were added and the reaction stirred at RT. After 2 h solid NaOH (290 mg, 7.3 mmol) was added, followed by 5 mL THF, and the cloudy reaction stirred over night. The reaction was then heated to reflux for 2 h and the solvent was removed under reduced pressure. The gummy residue was dissolved in 15 mL H₂O, and the pH adjusted to 6-6.5 by adding 6N HCl (3.8 mL). The H₂O was removed under reduced pressure and the resulting material removed of residual H₂O by azetropic distillation with MeOH. The addition and subsequent removal of MeOH under reduced pressure was performed several times and the resulting pale yellow solid was put under high vacuum. To this was added 15 mL MeOH and the solution decanted from the insoluble material. The solution was removed of solvent under reduced pressure to give 2.45 g 8C as a semi pure tan foam (150%). m/z (ES+) M+1=240; HPLC t_{R}=0.53 min.

### 8D. 1-cyclobutylpiperazine dihydrochloride

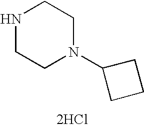

To tert-butyl 4-cyclobutylpiperazine-1-carboxylate (6.19 g, 25.75 mmol) prepared according to Zaragoza, et. al., J. Med. Chem. 2004, 47, 2833-2838 was added EtOAc (50 mL) and the solution cooled in ice bath. HCl gas was then bubbled in causing the HCl salt to immediately precipitate. MeOH was added and the reaction became homogeneous. HCl was bubbled in for 10 min and the reaction was allowed to warm to RT. After stirring for 1.5 h the reaction was diluted out with 500 mL Et₂O, stirred 30 min, filtered, and 8D placed under high vacuum to give 3.61 g (97%). m/z (ES+) M+1=141; HPLC t_{R}=0.24 min, ¹H NMR (300.132 MHz, DMSO-d₆/TFA-d) δ 1.68-1.87 (m, 2H), 2.18-2.40 (m, 4H), 3.11-3.57 (m, 8H), 3.81 (quintet, J=8.3 Hz, 1H).

### 8E. (4-Cyclobutylpiperazin-1-yl)(6-(tetrahydro-2H-pyran-4-yl)-6-azaspiro[2.5]octan-1-yl)methanone

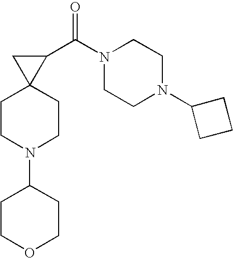

To semi pure 8C (150 mg, 0.63 mmol) was added HBTU (238 mg, 0.63 mmol), DMF (2.0 mL), and DIPEA (0.328 mL, 1.88 mmol) and the reaction stirred 5 min. To a separate vial was added 8D (160 mg, 0.75 mmol), DMF (1.0 mL), and DIPEA (0.263 mL, 1.50 mmol). This solution was added to the first reaction and the yellow solution stirred at RT. After 1.5 h the reaction was diluted with EtOAc (5 mL) and organic layer separated. The aq. layer was washed three times with DCM and this combined with the first organic layer and this washed 3 times with sat. NaHCO₃, 2 times with sat. NaCl, dried over Na₂SO₄, solvent removed under reduced pressure and the residue put under high vacuum. The crude material was chromatographed on 20 g silica with 10% (7N NH₃/MeOH)/DCM and the combined purified fractions removed of solvent under reduced pressure. The solid was triturated with Et₂O and the resulting precipitate was collected by filtration to give 77 mg 8E as a white solid (34%). m/z (ES+) M+1=362; HPLC t_{R}=0.26 min, HRMS (TOF) m/z calcd for C₂₁H₃₅N₃O₂[M+H]⁺, 362.2802. found, 362.2808; ¹H NMR (500.333 MHz, CDCl₃) δ 0.71 (dd, J=7.8, 4.4 Hz, 1H), 1.24 (t, J=4.8 Hz, 1H), 1.35-1.45 (m, 2H), 1.56-1.75 (m, 9H), 1.84-1.92 (m, 2H), 2.01-2.07 (m, 2H), 2.18-2.62 (m, 8H), 2.67-2.76 (m, 2H), 3.37 (t, J=11.2 Hz, 2H), 3.52 (m, 1H), 3.64 (t, J=4.7 Hz, 2H), 3.75 (m, 1H), 4.02 (dd, J=11.2, 4.1 Hz, 2H).

### Example 9

### (4-Cyclopropylpiperazin-1-yl)(6-(tetrahydro-2H-pyran-4-yl)-6-azaspiro[2.5]octan-1-yl)methanone

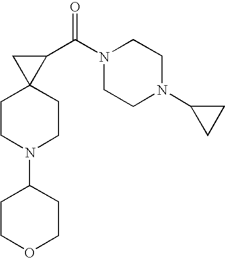

This example was prepared according to Example 8 using 8C and 1-cyclopropylpiperazine dihydrochloride which was prepared according to Gillaspy, et. al. Tet Lett. 1995, 36, 41, 7399-7402 and 8D. m/z (ES+) M+1=348; HPLC t_{R}=0.26 min, HRMS (TOF) m/z calcd for C₂₀H₃₃N₃O₂[M+H]⁺, 348.2646. found, 348.2654. ¹H NMR (500.333 MHz, CDCl₃) δ 0.41-0.45 (m, 2H), 0.46-0.50 (m, 2H), 0.69-0.73 (m, 1H), 1.24 (t, J=4.8 Hz, 1H), 1.35-1.46 (m, 2H), 1.57-1.66 (m, 5H), 1.69-1.76 (m, 3H), 2.43-2.72 (m, 9H), 3.37 (t, J=11.2 Hz, 2H), 3.49 (m, 1H), 3.59 (s, 2H), 3.69 (m, 1H), 4.02 (dd, J=11.1, 4.1 Hz, 2H).

### Example 10

### (4-Isopropylpiperazin-1-yl)(6-(tetrahydro-2H-pyran-4-yl)-6-azaspiro[2.5]octan-1-yl)methanone

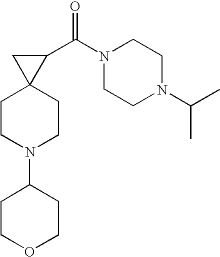

This example was prepared according to Example 8 using 8C and commercially available 1-isopropylpiperazine. m/z (ES+) M+1=350; HPLC t_{R}=0.26 min, HRMS (TOF) m/z calcd for C₂₀H₃₅N₃O₂[M+H]⁺, 350.2802. found, 350.2799. ¹H NMR (500.333 MHz, CDCl₃) δ 0.71 (dd, J=7.8, 4.4 Hz, 1H), 1.04 (d, J=6.5 Hz, 6H), 1.24 (t, J=4.8 Hz, 1H), 1.36-1.47 (m, 2H), 1.58-1.67 (m, 5H), 1.69-1.76 (m, 2H), 2.39-2.59 (m, 8H), 2.68-2.73 (m, 2H), 3.37 (t, J=11.6 Hz, 2H), 3.51 m, 1H), 3.63 (s, 2H), 3.76 (m, 1H), 4.02 (dd, J=11.3, 4.3 Hz, 2H).

### Example 11

### (4-Cycloheptylpiperazin-1-yl)(6-(tetrahydro-2H-pyran-4-yl)-6-azaspiro[2.5]octan-1-yl)methanone

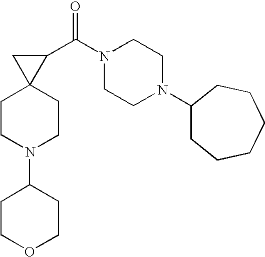

This example was prepared according to Example 8 using 8C and commercially available 1-cycloheptylpiperazine. m/z (ES+) M+1=404; HPLC t_{R}=0.26 min, HRMS (TOF) m/z calcd for C₂₄H₄₁N₃O₂ [M+H]⁺, 404.3272. found, 404.3278. ¹H NMR (500.333 MHz, CDCl₃) δ 0.70 (dd, J=7.8, 4.4 Hz, 1H), 1.24 (t, J=4.8 Hz, 1H), 1.37-1.82 (m, 21H), 2.39-2.60 (m, 9H), 2.67-2.71 (m, 1H), 3.37 (t, J=11.7 Hz, 2H), 3.46 m, 1H), 3.65 (s, 2H), 3.76 (m, 1H), 4.02 (dd, J=11.2, 4.1 Hz, 2H).

### Example 12

### (4-cyclopentylpiperazin-1-yl)(6-(tetrahydro-2H-pyran-4-yl)-6-azaspiro[2.5]octan-1-yl)methanone

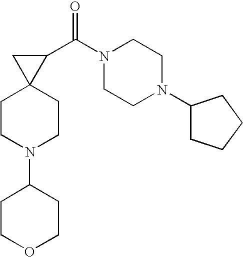

This example was prepared according to Example 8 using 8C and 1-cyclopentylpiperazine dihydrochloride, which was prepared according to Zaragoza, et. al. J. Med. Chem. 2004, 47, 2833-2838 and 8D. m/z (ES+) M+1=376; HPLC t_{R}=0.27 min; HRMS (TOF) m/z calcd for C₂₂H₃₇N₃O₂ [M+H]⁺, 376.2959. found, 376.2957. ¹H NMR (500.333 MHz, CDCl₃) δ 0.71 (dd, J=7.8, 4.4 Hz, 1H), 1.24 (t, J=4.8 Hz, 1H), 1.37-1.48 (m, 3H), 1.55-1.76 (m, 12H), 1.83-1.88 (m, 2H), 2.36-2.62 (m, 9H), 2.70-2.74 (m, 1H), 3.37 (t, J=11.0 Hz, 2H), 3.52 (m, 1H), 3.65 (t, J=4.8 Hz, 2H), 3.76 (m, 1H), 4.03 (dd, J=11.3, 4.1 Hz, 2H).

### Example 13

### (4-Pyridin-2-yl-piperazin-1-yl)-[6-(tetrahydro-pyran-4-yl)-6-aza-spiro[2.5]oct-1-yl]-methanone

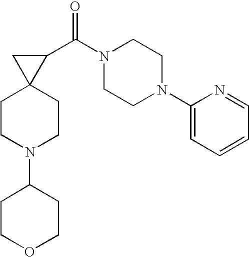

This example was prepared according to Example 8 using 8C and commercially available 1-(2-pyridyl)piperazine. m/z (ES+) M+1=385; HPLC t_{R}=0.16 min, HRMS (TOF ES+) m/z calc'd for C₂₂H₃₂N₄O₂ [M+H]⁺, 385.2598. found, 385.2583. ¹H NMR (500 MHz, DMSO-d₆) δ ppm 0.64-0.70 (m, 1H), 0.65-0.69 (m, 1H), 1.31-1.52 (m, 6H), 1.58-1.66 (m, 2H), 1.82-1.87 (m, 1H), 2.27-2.47 (m, 3H), 2.54 (br. s., 2H), 3.23 (t, J=11.7 Hz, 2H), 3.36-3.88 (m, 10H), 6.67 (dd, J=7.0, 4.9 Hz, 1H), 6.84 (d, J=8.5 Hz, 1H), 7.55 (m, 1H), 8.12 (d, J=1.8 Hz, 1H).

### Example 14

### (4-Pyridin-4-yl-piperazin-1-yl)-[6-(tetrahydro-pyran-4-yl)-6-aza-spiro[2.5]oct-1-yl]-methanone

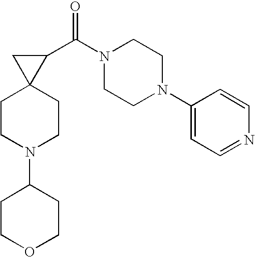

This example was prepared according to Example 8 using 8C and commercially available 1-(4-pyridyl)piperazine. m/z (ES+) M+1=385; HPLC t_{R}=0.14 min, HRMS (TOF ES+) m/z calc'd for C₂₂H₃₂N₄O₂ [M+H]⁺, 385.2598. found, 385.2598. ¹H NMR (500 MHz, DMSO-d₆) δ ppm 0.68 (dd, J=7.6, 4.0 Hz, 1H), 1.01 (t, J=4.9 Hz, 1H), 1.31-1.51 (m, 6H), 1.62 (dd, J=15.1, 2.3 Hz, 2H), 1.85 (dd, J=7.6, 5.2 Hz, 1H), 2.26-2.47 (m, 3H), 2.54 (m, 2H), 3.18-3.26 (m, 2H), 3.31-3.88 (m, 10H), 6.83 (d, J=6.7 Hz, 2H), 8.18 (d, J=6.1 Hz, 2H).

### Example 15

### [4-(4-Methoxy-phenyl)-piperazin-1-yl]-[6-(tetrahydro-pyran-4-yl)-6-aza-spiro[2.5]oct-1-yl]-methanone

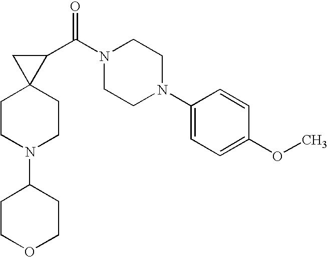

This example was prepared according to Example 8 using 8C and commercially available 1-(4-methoxyphenyl)piperazine. m/z (ES+) M+1=414; HPLC t_{R}=11.14 min; ¹H NMR (500 MHz, DMSO-d₆) δ ppm 0.67 (dd, J=7.8, 3.5 Hz, 1H), 1.00 (t, J=4.3 Hz, 1H), 1.31-1.51 (m, 6H), 1.63 (br. s., 2H), 1.85 (t, J=6.4 Hz, 1H), 2.29-2.47 (m, 3H), 2.52-2.59 (m, 2H), 2.87 (br. s., 1H), 2.99 (br. s., 2H), 3.09-3.17 (m, 1H), 3.20-3.25 (m, 2H), 3.54 (br. s., 1H), 3.69 (s, 4H), 3.84 (m, 4H), 6.83 (d, J=9.2 Hz, 2H), 6.89-6.94 (m, 2H).

### Example 16

### (4-Cyclohexyl-piperazin-1-yl)-(6-phenethyl-6-aza-spiro[2.5]oct-1-yl)-methanone

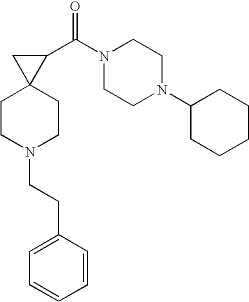

### 16A. Benzyl 4-oxopiperidine-1-carboxylate

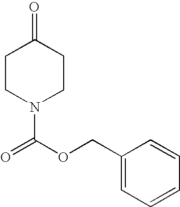

A mixture of 4-piperidone monohydrate hydrochloride (100 g, 0.651 mol), benzyl chloroformate (112 mL, 0.781 mol) and NaHCO₃ (164 g, 1.95 mol) in dioxane (800 mL) and H₂O (670 mL) was stirred at RT for 48 h. H₂O (1 L) was added and the mixture was extracted with CH₂Cl₂ (1×1 L, 2×300 mL). The organic layer was washed with brine and dried over MgSO₄. Upon evaporation, 173 g 16A was obtained as a colorless liquid (100%). ¹H NMR (300 MHz, CDCl₃): δ 2.43-2.48 (4H, m), 3.77-3.82 (4H, m), 5.18 (2H, s), 7.34-7.39 (5H, m). Alternately, this material can be purchased from commercial sources.

### 16B. Benzyl 4-(2-ethoxy-2-oxoethylidene)piperidine-1-carboxylate

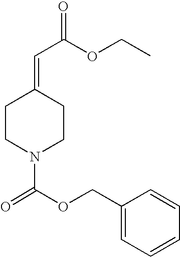

### Method 1:

A mixture of 16A (11.4 g, 46.5 mmol) and (ethoxycarbonylmethylene)triphenylphosphorane (21.67 g, 62.2 mmol) in toluene (150 mL) was heated at reflux for 18 h. It was concentrated and the residue was purified by flash-chromatography (hexanes:EtOAc 4:1) to give 12.80 g 16B as a colorless liquid (91%). ¹H NMR (300 MHz, CDCl₃): δ 1.27 (3H, t, J=7.1 Hz), 2.30 (2H, t, J=4.5 Hz), 2.96 (2H, t, J=5.5 Hz), 3.57 (4H, dd, J=12.2, 6.5 Hz), 4.15 (2H, q, J=7.1 Hz), 5.15 (2H, s), 5.72 (1H, s), 7.31-7.38 (5H, m).

### Method 2:

To ethyl 2-(diethoxyphosphoryl)acetate (4.68 mL, 23.58 mmol) in THF (50 mL) at −78° C. was added 2.5M BuLi hexanes (9.43 mL, 23.58 mmol) and the reaction stirred 10 min. To this mixture was added solid 16A (5.00 g, 21.44 mmol) and the reaction allowed to warm to RT and stirred 30 min. The solvent was removed and resulting material placed under high vacuum. The resulting solids were triturated with 100 mL hexanes and the mixture filtered through Celite. The filtrate was removed of solvent and the resulting oil placed under high vacuum over night to give 5.52 g 16B as a semi pure clear oil (85%). m/z (ES+) M+1=not observed; HPLC t_{R}=2.31 min, ¹H NMR (300 MHz, CDCl₃) δ ppm 1.28 (t, J=6.95 Hz, 3H) 2.30 (t, J=5.27 Hz, 2H) 2.96 (t, J=5.69 Hz, 2H) 3.57 (q, J=6.32 Hz, 4H) 4.15 (q, J=7.17 Hz, 2H) 5.15 (s, 2H) 5.72 (s, 1H) 7.28-7.47 (m, 5H).

### 16C. 6-Benzyl 1-ethyl 6-azaspiro[2.5]octane-1,6-dicarboxylate

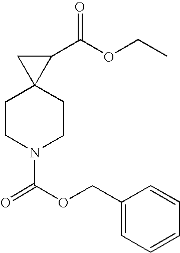

### Method 1:

Potassium tert-butoxide (8.43 g, 75.1 mmol) was added to a solution of trimethylsulfoxonium iodide (17.5 g, 79.5 mmol) in DMSO (100 mL) in one portion. The reaction mixture was stirred at RT for 2 h. 16B (13.4 g, 44.2 mmol) in DMSO (50 mL) was added to the reaction mixture. The mixture was stirred at RT overnight and then added slowly to sat. NH₄Cl at 0° C. with stirring. The mixture was extracted with ether. The organic layer was washed with sat. NaHCO₃ and concentrated under reduced pressure. The residue was dissolved in EtOAc (100 mL), and stirred with aq. KMnO₄ (2.0 g in 200 mL H₂O) and NaHCO₃ (1.5 g) overnight. This was filtered through Celite and washed with EtOAc. The organic layer was washed with brine and dried over Na₂SO₄. The material was purified with flash-chromatography (hexanes:EtOAc 5:1 to 4:1) to afford 10.52 g 16C as a colorless liquid (75%). ¹H NMR (300 MHz, CDCl₃): δ 0.93 (1H, dd, J=8.0, 4.6 Hz), 1.17 (1H, t, J=5.0 Hz), 1.26 (3H, t, J=7.1 Hz), 1.40-1.46 (2H, m), 1.56 (1H, dd, J=8.0, 5.4 Hz), 1.69-1.74 (2H, m), 3.31-3.39 (1H, m), 3.46-3.61 (3H, m), 4.13 (2H, q, J=7.1 Hz), 5.13 (2H, s), 7.30-7.36 (5H, m). ¹³C NMR (75 MHz, CDCl₃): δ 14.6, 19.9, 25.5, 28.5, 28.6, 36.4, 43.9, 44.2, 60.7, 67.3, 128.1, 128.2, 128.7, 137.1, 155.5, 172.3.

### Method 2:

To hexanes washed 60% NaH (1.978 g, 49.45 mmol) was added solid trimethylsulfoxonium iodide (11.24 g, 51.10 mmol) followed by careful addition of DMSO (40 mL). After 30 min the fizzing stopped and a solution of 16B (5.00 g, 16.48 mmol) in 10 mL DMSO was added and the reaction left to stir at RT over the weekend. The reaction was partitioned between EtOAc/H₂O with a small amount of brine to aid partitioning of the layers. The aq. was washed with EtOAc and the combined organics washed 5×H₂O, 1×NaHCO₃, 1×NaCl, dried over Na₂SO₄, solvent removed under reduced pressure, and the oil placed under high vacuum. The material was chromatographed on 70 g silica with 20% EtOAc/Hexane. The fractions were removed of solvent under reduced pressure and the material put under high vacuum to give 3.186 g 16C as a clear, semi pure, oil (61%). m/z (ES+) M+1=not observed; HPLC t_{R}=5.71 min, ¹H NMR (500 MHz, CDCl₃) δ ppm 0.93 (dd, J=7.93, 4.88 Hz, 1H) 1.17 (t, J=4.88 Hz, 1H) 1.26 (t, J=7.02 Hz, 3H) 1.44 (d, J=4.27 Hz, 2H) 1.56 (dd, J=7.93, 5.49 Hz, 1H) 1.72 (d, J=4.27 Hz, 2H) 3.36 (dd, J=7.32, 4.27 Hz, 1H) 3.45-3.62 (m, 3H) 4.13 (q, J=6.71 Hz, 2H) 5.13 (s, 2H) 7.27-7.39 (m, 5H).

### 16D. 6-Aza-spiro[2.5]octane-1,6-dicarboxylic acid 6-benzyl ester

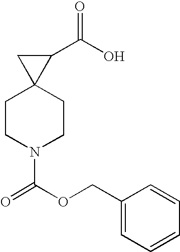

To 16C (3.19 g, 10.04 mmol) in MeOH (50 mL) was added H₂O (5 mL) and 1N NaOH (20.08 mL, 20.08 mmol). The reaction was left to stir at RT over night then was heated to reflux for 2 h. The MeOH was removed and EtOAc/1N HCl added. The aq. layer was washed 3×EtOAc and combined organic layers washed 1×NaCl, dried over Na₂SO₄, and the solvent removed under reduced pressure to give 2.999 g 16D as a clear gum (103%). m/z (ES+) M+1=290; HPLC t_{R}=5.09 min, ¹H NMR (500 MHz, CDCl₃) δ ppm 1.02 (dd, J=7.63, 4.58 Hz, 1H) 1.18-1.25 (m, 2H) 1.45 (br. s., 2H) 1.58 (d, J=5.49 Hz, 1H) 1.76 (br. s., 2H) 2.09 (s, 0H) 3.38-3.61 (m, 4H) 5.14 (s, 2H) 7.28-7.40 (m, 5H).

### 16E. 1-(4-Cyclohexyl-piperazine-1-carbonyl)-6-aza-spiro[2.5]octane-6-carboxylic acid benzyl ester

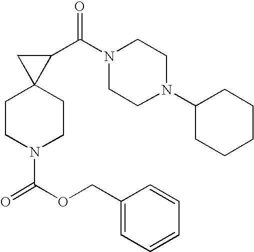

To 16D (2.99 g, 10.33 mmol) was added, DMF (40 mL), HBTU (3.92 g, 10.33 mmol), DIPEA (5.41 mL, 31.00 mmol), and mixture stirred 5 min. To this was added commercially available 1-cyclohexylpiperazine (1.913 g, 11.37 mmol) and the reaction stirred for 2 h. The DMF was partially removed under reduced pressure and remaining material partitioned between EtOAc/NaHCO₃. The organic layer was washed 3×NaHCO₃, 1×NaCl, dried over Na₂SO₄, removed of solvent and sticky solids put under high vacuum. The material chromatographed on 120 g silica with 2.5% MeOH/DCM. A heart cut in fractions was taken and solvent removed under reduced pressure to give batch 1 of 2.14 g of 16E as a pale white solid. The impure lower fractions were removed of solvent under reduced pressure, 1.5 g, and chromatographed on 50 g silica with 2.5% (7N NH₃/MeOH)/DCM and the combined purified fractions were removed of solvent to give batch 2 of 1.07 g 16E as an off-white solid, which was combined with batch 1 (3.21 g, 71%). m/z (ES+) M+1=440; HPLC t_{R}=1.93 min, ¹H NMR (500 MHz, CDCl₃) δ ppm 0.77 (dd, J=7.63, 4.58 Hz, 1H) 1.05-1.37 (m, 7H) 1.42 (d, J=14.65 Hz, 1H) 1.63 (dd, J=7.93, 5.49 Hz, 3H) 1.80 (d, J=15.26 Hz, 4H) 2.22-2.34 (m, 2H) 2.47 (dd, J=17.70, 10.99 Hz, 2H) 2.53-2.70 (m, 2H) 3.31 (dd, J=13.43, 5.49 Hz, 1H) 3.39 (ddd, J=12.97, 9.00, 3.66 Hz, 1H) 3.49 (br. s., 1H) 3.60 (br. s., 3H) 3.71 (dd, J=7.93, 4.88 Hz, 2H) 5.14 (s, 2H) 7.36 (d, J=4.27 Hz, 5H).

Alternate purification: If impure 16D, prepared from 16C method 2, was used to prepare intermediates of this nature, they were purified by reverse phase HPLC on an Agilent Technologies 1100 Prep-HPLC using a linear gradient of H₂O with 0.1% TFA (solvent A) and ACN with 0.1% TFA (solvent B); t=0 min, 0% B to t=12 min, 50% B to t=20 min, 100% B. The purification was performed using a Phenomenex Luna, 10μ, C18, 21×250 mm column with a flow rate of 40 mL/min and collecting at 220 nm A heart cut of the fractions was taken to obtain clean material. The fractions were removed of solvent under reduced pressure and the material partitioned between EtOAc/NaHCO₃. The organic layer was washed 1×NaCl, dried over Na₂SO₄, and the solvent removed under reduced pressure to give purified materials.

### 16F. (6-Aza-spiro[2.5]oct-1-yl)-(4-cyclohexyl-piperazin-1-yl)-methanone

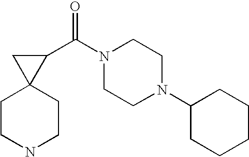

To 16E (3.21 g, 7.30 mmol) in MeOH (25 mL) was added 5% Pd/C (0.311 g, 0.15 mmol), and a balloon of hydrogen fixed atop the reaction. The reaction was stirred at RT for 2 h and then filtered through Celite. The filtrate was removed of solvent under reduced pressure and the material put under high vacuum to give 2.126 g 16F as a white solid (95%). m/z (ES+) M+1=306; HPLC t=0.51 min, HRMS (TOF ES+ ES+) m/z calc'd for C₁₈H₃₁N₃O₂ [M+H]⁺, 306.2540. found, 306.2542. ¹H NMR (500 MHz, CDCl₃) δ ppm 0.72 (dd, J=7.93, 4.27 Hz, 1H) 1.04-1.17 (m, 1H) 1.17-1.27 (m, 4H) 1.27-1.34 (m, 1H) 1.34-1.42 (m, 1H) 1.51-1.59 (m, 2H) 1.62 (br. s., 4H) 1.76-1.90 (m, 4H) 2.23-2.33 (m, 1H) 2.45 (t, J=8.24 Hz, 1H) 2.49-2.61 (m, 2H) 2.62-2.70 (m, 1H) 2.74-2.84 (m, 2H) 2.84-2.98 (m, 2H) 3.42-3.53 (m, 1H) 3.57-3.71 (m, 2H) 3.73-3.83 (m, 1H).

### 16G. (4-Cyclohexyl-piperazin-1-yl)-(6-phenethyl-6-aza-spiro[2.5]oct-1-yl)-methanone

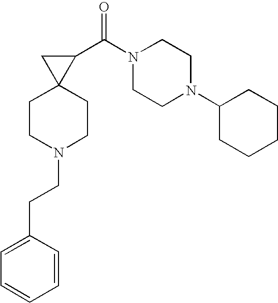

To 16F (100 mg, 0.33 mmol) was added DCE (2 mL) and 2-phenylacetaldehyde (37 μL, 0.33 mmol) and after the reaction stirred for 30 min, sodium triacetoxyborohydride (76 mg, 0.36 mmol) was added. After stirring at RT over night, the reaction was quenched with 1.5 mL 1N NaOH and the mixture stirred 20 min. The aq. layer was removed and Et₂O/sat. NaCl added to the organic layer and stirred 5 min. The aq. layer was removed and the organic layer dried over Na₂SO₄, solvent removed under reduced pressure, and material put under high vacuum 50° C. over night. Material was purified on 12 g silica gel eluting with DCM to 10% (7N NH₃/MeOH)/DCM over 11 min at 30 mL/min. The combined purified fractions were removed of solvent under reduced pressure and the material put under high vacuum. The material was dissolved in hot hexanes, cooled in freezer, and the solids collected and put under high vacuum 50° C. over night to give 85 mg 16G as a white solid (63%). m/z (ES+) M+1=410; HPLC t_{R}=3.57 min, HRMS (TOF ES++ES+) m/z calc'd for C₂₆H₃₉N₃O₂ [M+H]⁺, 410.3166. found, 410.3169. ¹H NMR (300 MHz, CDCl₃) δ ppm 0.72 (dd, J=7.80, 4.43 Hz, 1H) 1.01-1.34 (m, 6H) 1.44 (m, 2H) 1.55-1.75 (m, 5H) 1.75-1.92 (m, 4H) 2.19-2.33 (m, 1H) 2.35-2.73 (m, 9H) 2.75-2.92 (m, 2H) 3.38-3.54 (m, 1H) 3.64 (br. s., 2H) 3.70-3.89 (m, 1H) 7.15-7.34 (m, 5H).

Alternate purification: compounds prepared in this fashion were also purified by reverse phase chromatography on a 30 mm×100 mm, 5 u Gemini C18 column at pH 10 [NH₄HCO₃] with ACN/H₂O as the mobile phase. Solvent flow was 50 ml/min, and the gradient runs from 5 to 95% ACN over 10 or 20 min Fractions were collected by MS detection and were removed of solvent using a Genevac Series II rotary evaporator. The resulting solids were redissolved in ACN/H₂O and lyophilized to give the purified material.

### Example 17

### (4-Cyclohexyl-piperazin-1-yl)-(6-phenethyl-6-aza-spiro[2.5]oct-1-yl)-methanone, enantiomer 1

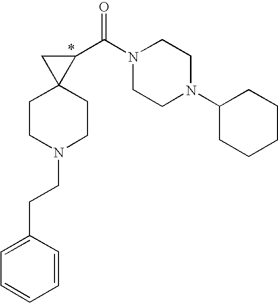 [0391] Note: * designates single enantiomer of unknown stereochemistry.

16G (79 mg, 0.19 mmol) was separated into individual enantiomers on a Berger Instruments MultiGram II Supercritical Fluid Chromatography Instrument using the following conditions: 21×250 mm ChiralPak AD-H, 5 micron column, 50.0 mL/min, 20:80 (isopropanol containing 0.5% dimethylethylamine): supercritical CO₂, UV-220 nm. The isolated enantiomer was removed of solvent under reduced pressure and placed under high vacuum to give 36.1 mg title compound as a white solid (46%). m/z (TOF ES+) M+1=410; HPLC t_{R}=0.55 min, HRMS (TOF ES+) m/z calc'd for C₂₆H₃₉N₃O₂ [M+H]⁺, 410.3166. found, 410.3155. ¹H NMR (500 MHz, DMSO-d₆) δ ppm 0.65 (dd, J=7.93, 3.66 Hz, 1H) 0.98 (t, J=4.27 Hz, 1H) 1.01-1.12 (m, 1H) 1.12-1.25 (m, 4H) 1.31-1.40 (m, 1H) 1.40-1.51 (m, 3H) 1.56 (d, J=12.21 Hz, 1H) 1.73 (br. s., 4H) 1.76-1.82 (m, 1H) 2.20-2.31 (m, 2H) 2.31-2.38 (m, 1H) 2.39-2.55 (m, 8H) 2.58 (br. s., 1H) 2.72 (t, J=7.63 Hz, 2H) 3.57 (m, 3H) 7.14-7.23 (m, 3H) 7.25 (m, 2H): Analytical Chiral SFC analysis of final target >99% ee, t_{R}=7.39 min, on 4.6×250 mm ChiralPak AD-H, 5 micron column, 2.37 mL/min, 20:80 (isopropanol containing 0.5% dimethylethylamine): supercritical CO₂, UV-DAD and MS detection.

### Example 18

### (4-Cyclohexyl-piperazin-1-yl)-(6-phenethyl-6-aza-spiro[2.5]oct-1-yl)-methanone, enantiomer 2

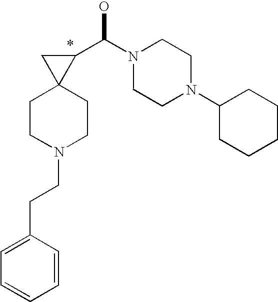 [0394] Note: * designates single enantiomer of unknown stereochemistry.

This enantiomer was isolated in accordance with the chiral separation described in Example 17 and treated as described for that example. 29.3 mg title compound was isolated as a white solid (37%). m/z (TOF ES+) M+1=410; HPLC t_{R}=0.56 min, HRMS (TOF ES+) m/z calc'd for C₂₆H₃₉N₃O₂ [M+H]⁺, 410.3166. found, 410.3170. ¹H NMR (500 MHz, DMSO-d₆) δ ppm 0.65 (dd, J=7.93, 3.66 Hz, 1H) 0.98 (t, J=4.27 Hz, 1H) 1.01-1.12 (m, 1H) 1.13-1.25 (m, 4H) 1.32-1.40 (m, 1H) 1.45 (dd, J=9.46, 3.36 Hz, 3H) 1.56 (d, J=11.60 Hz, 1H) 1.73 (br. s., 4H) 1.77-1.81 (m, 1H) 2.21-2.31 (m, 2H) 2.31-2.38 (m, 1H) 2.39-2.56 (m, 8H) 2.56-2.63 (m, 1H) 2.72 (t, J=7.63 Hz, 2H) 3.53-3.66 (m, 3H) 7.14-7.23 (m, 3H) 7.25 (d, J=7.32 Hz, 2H); Analytical Chiral SFC analysis of final target >99% ee, t_{R}=9.09 min, on 4.6×250 mm ChiralPak AD-H, 5 micron column, 2.37 mL/min, 20:80 (isopropanol containing 0.5% dimethylethylamine): supercritical CO₂, UV-DAD and MS detection.

### Example 19

### (6-Cyclohexyl-6-aza-spiro[2.5]oct-1-yl)-(4-cyclohexyl-piperazin-1-yl)-methanone

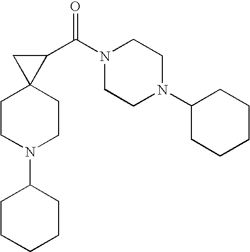

This example was prepared according to Example 16 using 16F and commercially available cyclohexanone. m/z (TOF ES+) M+1=388; HPLC t_{R}=0.43 min, HRMS (TOF ES+) m/z calc'd for C₂₄H₄ᵢN₃O [M+H]⁺, 388.3322. found, 388.3329. ¹H NMR (300 MHz, CDCl₃) δ ppm 0.69 (dd, J=7.80, 4.43 Hz, 1H) 1.00-1.32 (m, 11H) 1.42 (d, J=14.75 Hz, 2H) 1.51-1.72 (m, 5H) 1.80 (d, J=9.27 Hz, 8H) 2.20-2.38 (m, 2H) 2.39-2.73 (m, 8H) 3.39-3.54 (m, 1H) 3.63 (br. s., 2H) 3.74 (d, J=2.53 Hz, 1H).

### Example 20

### (4-Cyclohexyl-piperazin-1-yl)-[6-(1-methyl-piperidin-4-yl)-6-aza-spiro[2.5]oct-1-yl]-methanone

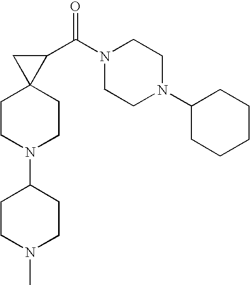

This example was prepared according to Example 16 using 16F and commercially available 1-methylpiperidin-4-one. m/z (TOF ES+) M+1=402; HPLC t_{R}=0.12 min, HRMS (TOF ES+) m/z calc'd for C₂₄H₄₂N₄O [M+H]⁺, 403.3431. found, 403.3436. ¹H NMR (300 MHz, CDCl₃) δ ppm 0.69 (dd, J=7.80, 4.43 Hz, 1H) 1.22 (d, J=7.59 Hz, 6H) 1.31-1.49 (m, 2H) 1.50-2.00 (m, 14H) 2.16-2.37 (m, 5H) 2.38-2.73 (m, 8H) 2.90 (d, J=11.38 Hz, 2H) 3.34-3.53 (m, 1H) 3.63 (br. s., 2H) 3.70-3.87 (m, 1H).

### Example 21

### (4-Cyclohexyl-piperazin-1-yl)-(6-isopropyl-6-aza-spiro[2.5]oct-1-yl)-methanone

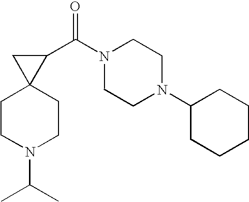

This example was prepared according to Example 16 using 16F and commercially available 2-propanone. m/z (TOF ES+) M+1=348; HPLC t_{R}=0.16 min, HRMS (TOF ES+) m/z calc'd for C₂₁H₃₇N₃O [M+H]⁺, 348.3009. found, 348.3010. ¹H NMR (300 MHz, CDCl₃) δ ppm 0.69 (dd, J=7.59, 4.22 Hz, 1H) 1.05 (d, J=6.74 Hz, 6H) 1.10-1.30 (m, 6H) 1.31-1.50 (m, 2H) 1.50-1.75 (m, 5H) 1.75-1.90 (m, 4H) 2.20-2.33 (m, 1H) 2.33-2.67 (m, 7H) 2.67-2.83 (m, 1H) 3.38-3.54 (m, 1H) 3.63 (br. s., 2H) 3.70-3.87 (m, 1H).

### Example 22

### (6-Cyclobutyl-6-aza-spiro[2.5]oct-1-yl)-[4-(tetrahydro-pyran-4-yl)-piperazin-1-yl]-methanone

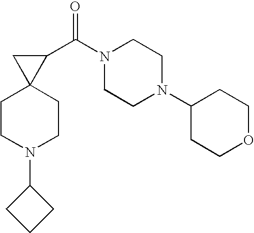

This example was prepared according to Example 16 using 16D, 1-(tetrahydro-pyran-4-yl)-piperazine which was prepared according to 8D, and commercially available cyclobutanone. m/z (ES+) M+1=362; HPLC t_{R}=0.19 min; ¹H NMR (500 MHz, CDCl₃) δ ppm 0.71 (dd, J=7.63, 4.58 Hz, 1H) 1.24 (t, J=4.88 Hz, 1H) 1.27-1.47 (m, 2H) 1.50-1.80 (m, 9H) 1.82-1.95 (m, 2H) 1.97-2.08 (m, 2H) 2.08-2.27 (m, 2H) 2.34-2.80 (m, 8H) 3.38 (t, J=11.60 Hz, 2H) 3.43-3.55 (m, 1H) 3.64 (br. s., 2H) 3.72-3.84 (m, 1H) 4.02 (d, J=3.05 Hz, 2H).

### Example 23

### (6-Benzyl-6-aza-spiro[2.5]oct-1-yl)-(4-cyclohexyl-piperazin-1-yl)-methanone

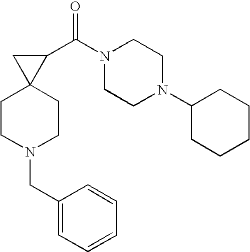

This example was prepared according to Example 16 using 16F and commercially available benzaldehyde. m/z (TOF ES+) M+1=396; HPLC t_{R}=0.40 min, HRMS (TOF ES+) m/z calc'd for C₂₅H₃₇N₃O [M+H]⁺, 396.3009. found, 396.3004. ¹H NMR (300 MHz, CDCl₃) δ ppm 0.69 (dd, J=7.80, 4.43 Hz, 1H) 1.00-1.31 (m, 5H) 1.31-1.50 (m, 2H) 1.51-1.72 (m, 6H) 1.73-1.93 (m, 4H) 2.19-2.70 (m, 8H) 3.41-3.55 (m, 3H) 3.60 (br. s., 2H) 3.66-3.80 (m, 1H) 7.19-7.39 (m, 5H).

### Example 24

### [6-(Tetrahydro-pyran-4-yl)-6-aza-spiro[2.5]oct-1-yl]-[4-(tetrahydro-pyran-4-yl)-piperazin-1-yl]-methanone

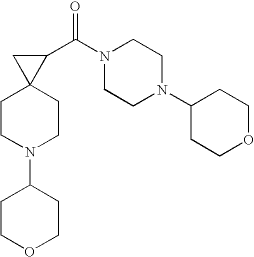

This example was prepared according to Example 16 using 16D, 1-(tetrahydro-pyran-4-yl)-piperazine, which was prepared according to Zaragoza, et. al. J. Med. Chem. 2004, 47, 2833-2838 and 8D, and commercially available tetrahydro-pyran-4-one. m/z (ES+) M+1=392; HPLC t_{R}=0.19 min; ¹H NMR (500 MHz, CDCl₃) δ ppm 0.72 (dd, J=7.63, 4.58 Hz, 1H) 1.25 (t, J=4.88 Hz, 1H) 1.30-1.40 (m, 1H) 1.41-1.50 (m, 1H) 1.51-1.69 (m, 7H) 1.70-1.85 (m, 4H) 2.30-2.85 (m, 10H) 3.37 (t, J=11.60 Hz, 4H) 3.45-3.57 (m, 1H) 3.64 (br. s., 2H) 3.71-3.83 (m, 1H) 4.03 (dd, J=10.99, 3.66 Hz, 4H).

### Example 25

### [6-(2-Benzyloxy-ethyl)-6-aza-spiro[2.5]oct-1-yl]-(4-cyclohexyl-piperazin-1-yl)-methanone

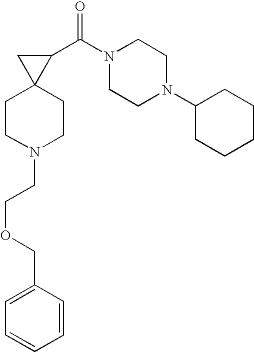

This example was prepared according to Example 16 using 16F and commercially available 2-(benzyloxy)acetaldehyde. m/z (TOF ES+) M+1=440; HPLC t_{R}=0.66 min, HRMS (TOF ES+) m/z calc'd for C₂₇H₄₁N₃O₂ [M+H]⁺, 440.3272. found, 440.3275. ¹H NMR (500 MHz, DMSO-d₆) δ ppm 0.63 (dd, J=7.93, 3.66 Hz, 1H) 0.96 (t, J=4.58 Hz, 1H) 1.01-1.11 (m, 1H) 1.12-1.25 (m, 4H) 1.27-1.36 (m, 1H) 1.37-1.48 (m, 3H) 1.56 (d, J=11.60 Hz, 1H) 1.67-1.80 (m, 5H) 2.18-2.29 (m, 2H) 2.29-2.41 (m, 2H) 2.42-2.55 (m, 7H, under DMSO) 2.55-2.63 (m, 1H) 3.50-3.65 (m, 5H) 4.46 (s, 2H) 7.23-7.39 (m, 5H).

### Example 26

### (4-Cyclobutyl-piperazin-1-yl)-(6-cyclohexyl-6-aza-spiro[2.5]oct-1-yl)-methanone, single enantiomer

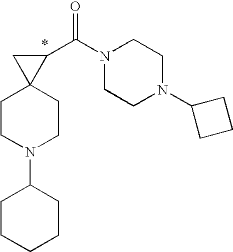 [0411] Note: * designates single enantiomer of unknown stereochemistry.

### 26A. 1-(4-Cyclobutyl-piperazine-1-carbonyl)-6-aza-spiro[2.5]octane-6-carboxylic acid benzyl ester, single enantiomer

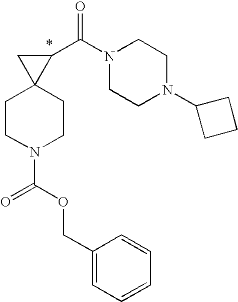 [0413] Note: * designates single enantiomer of unknown stereochemistry.

Racemic 1-(4-cyclobutyl-piperazine-1-carbonyl)-6-aza-spiro[2.5]octane-6-carboxylic acid benzyl ester, which was prepared from 16D and 8D in accordance with the process used in preparing 16E. The material (1.566 g) was separated into the individual enantiomers on a Berger Instruments MultiGram II Supercritical Fluid Chromatography Instrument using the following conditions: 21×250 mm ChiralPak AD-H, 5 micron column, 50.0 mL/min, 35:65 (MeOH containing 0.5% dimethylethylamine): supercritical CO₂, UV-220 nm 26A was removed of solvent under reduced pressure and placed under high vacuum to give 429 mg 26A as a white solid (40%). m/z (ES+) M+1=412; HPLC t_{R}=1.86 min; ¹H NMR (500 MHz, CDCl₃) δ ppm 0.78 (dd, J=7.93, 4.27 Hz, 1H) 1.22-1.37 (m, 2H) 1.38-1.47 (m, 1H) 1.52-1.65 (m, 3H) 1.65-1.79 (m, 2H) 1.81-1.93 (m, 2H) 1.99-2.09 (m, 2H) 2.11-2.27 (m, 2H) 2.31-2.48 (m, 2H) 2.73 (dq, J=7.93, 7.73 Hz, 1H) 3.26-3.34 (m, 1H) 3.34-3.42 (m, 1H) 3.50 (br. s., 1H) 3.62 (br. s., 3H) 3.67-3.85 (m, 2H) 5.14 (s, 2H) 7.28-7.38 (m, 5H); Analytical Chiral SFC analysis of final target >99% ee, t_{R}=3.70 min, on 4.6×250 mm ChiralPak AD-H, 5 micron column, 2.37 mL/min, 35:65 (MeOH containing 0.5% dimethylethylamine): supercritical CO₂, UV-DAD and MS detection.

### 26B. (4-Cyclobutyl-piperazin-1-yl)-(6-cyclohexyl-6-aza-spiro[2.5]oct-1-yl)-methanone, single enantiomer

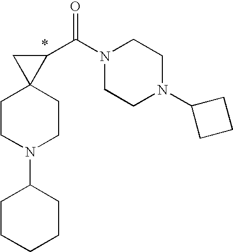

To 26A (250 mg, 0.61 mmol) was added MeOH (3 mL), cyclohexanone (0.252 mL, 2.43 mmol), Pd/C (12.93 mg, 0.01 mmol), and a balloon filled with H₂ (excess) fixed atop the reaction. After stirring over night the reaction was filtered, solvent removed, and chromatographed on 40 g silica DCM to 5% (7N NH₃/MeOH)/DCM. A heart cut in fractions was taken and the solvent removed under reduced pressure. The material was placed under high vacuum 50° C. over night to give 121 mg 26B as a white solid (55%). m/z (ES+) M+1=360; HPLC t_{R}=0.25 min; ¹H NMR (500 MHz, CDCl₃) δ ppm 0.69 (dd, J=7.63, 4.58 Hz, 1H) 1.02-1.15 (m, 1H) 1.16-1.30 (m, 5H) 1.31-1.46 (m, 2H) 1.50-1.94 (m, 12H) 1.99-2.08 (m, 2H) 2.15-2.23 (m, 1H) 2.24-2.37 (m, 3H) 2.37-2.43 (m, 1H) 2.43-2.50 (m, 1H) 2.55 (br. s., 2H) 2.62-2.79 (m, 2H) 3.46-3.56 (m, 1H) 3.64 (br. s., 2H) 3.70-3.82 (m, 1H); Analytical Chiral SFC analysis of final target >99% ee, t_{R}=5.57 min, on 4.6×250 mm ChiralPak AD-H, 5 micron column, 2.37 mL/min, 15:85 (MeOH containing 0.5% dimethylethylamine): supercritical CO₂, UV-DAD and MS detection.

### Example 27

### (4-Cyclobutyl-piperazin-1-yl)-(6-cyclohexyl-6-aza-spiro[2.5]oct-1-yl)-methanone, single enantiomer

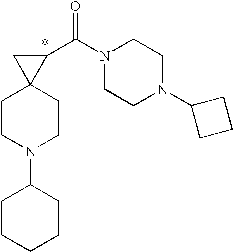 [0418] Note: * designates single enantiomer of unknown stereochemistry.

### 27A. 1-(4-Cyclobutyl-piperazine-1-carbonyl)-6-aza-spiro[2.5]octane-6-carboxylic acid benzyl ester, single enantiomer

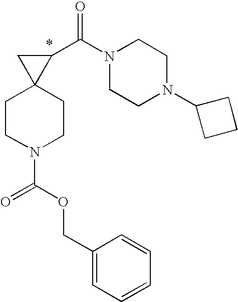

27A was collected as part of the SFC separations for 26A and was subsequently chromatographed on 40 g silica eluting DCM to 7.5% MeOH/DCM. The purified product was removed of solvent under reduced pressure to give 436 mg 27A as a white solid (41%). m/z (ES+) M+1=412; HPLC t_{R}=1.83 min; ¹H NMR (500 MHz, CDCl₃) δ ppm 0.78 (dd, J=7.63, 4.58 Hz, 1H) 1.23-1.36 (m, 2H) 1.37-1.47 (m, 1H) 1.51-1.66 (m, 3H) 1.67-1.79 (m, 2H) 1.81-1.93 (m, 2H) 1.98-2.09 (m, 2H) 2.12-2.26 (m, 2H) 2.31-2.48 (m, 2H) 2.72 (qd, J=7.83, 7.63 Hz, 1H) 3.25-3.34 (m, 1H) 3.34-3.42 (m, 1H) 3.50 (br. s., 1H) 3.62 (br. s., 3H) 3.67-3.84 (m, 2H) 5.14 (s, 2H) 7.28-7.39 (m, 5H); Analytical Chiral SFC analysis of final target >99% ee, t_{R}=5.35 min, on 4.6×250 mm ChiralPak AD-H, 5 micron column, 2.37 mL/min, 35:65 (MeOH containing 0.5% dimethylethylamine): supercritical CO₂, UV-DAD and MS detection.

### 27B. 4-Cyclobutyl-piperazin-1-yl)-(6-cyclohexyl-6-aza-spiro[2.5]oct-1-yl)-methanone, single enantiomer

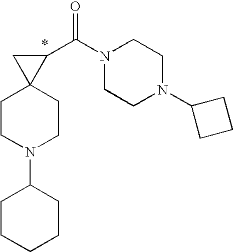

27B was prepared as described for 26B using 27A and commercially available cyclohexanone. m/z (ES+) M+1=360; HPLC t_{R}=0.32 min; ¹H NMR (500 MHz, CDCl₃) δ ppm 0.69 (dd, J=7.63, 4.58 Hz, 1H) 1.03-1.15 (m, 1H) 1.16-1.30 (m, 5H) 1.32-1.47 (m, 2H) 1.51-1.94 (m, 12H) 1.98-2.09 (m, 2H) 2.16-2.23 (m, 1H) 2.24-2.37 (m, 3H) 2.37-2.43 (m, 1H) 2.43-2.50 (m, 1H) 2.51-2.61 (m, 2H) 2.63-2.78 (m, 2H) 3.47-3.57 (m, 1H) 3.64 (br. s., 2H) 3.71-3.81 (m, 1H); Analytical Chiral SFC analysis of final target >99% ee, t_{R}=6.41 min, on 4.6×250 mm ChiralPak AD-H, 5 micron column, 2.37 mL/min, 15:85 (MeOH containing 0.5% dimethylethylamine): supercritical CO₂, UV-DAD and MS detection.

### Example 28

### (4-Isopropyl-piperazin-1-yl)-[6-(tetrahydro-pyran-4-yl)-6-aza-spiro[2.5]oct-1-yl]-methanone, single enantiomer

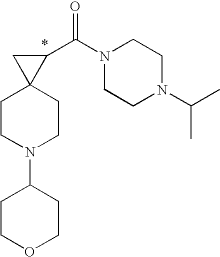 [0424] Note: * designates single enantiomer of unknown stereochemistry.

This compound was prepared according to Example 26 using 16D, commercially available 1-isopropylpiperazine, and commercially available tetrahydro-pyran-4-one. m/z (ES+) M+1=350; HPLC t_{R}=0.17 min; ¹H NMR (500 MHz, CDCl₃) δ ppm 0.71 (dd, J=7.93, 4.27 Hz, 1H) 1.04 (d, J=6.71 Hz, 6H) 1.24 (t, J=4.88 Hz, 1H) 1.32-1.41 (m, 1H) 1.41-1.49 (m, 1H) 1.54-1.67 (m, 4H) 1.67-1.79 (m, 3H) 2.35-2.63 (m, 8H) 2.65-2.77 (m, 2H) 3.37 (t, J=11.60 Hz, 2H) 3.46-3.56 (m, 1H) 3.63 (br. s., 2H) 3.71-3.81 (m, 1H) 4.02 (dd, J=11.29, 3.97 Hz, 2H); Analytical Chiral SFC analysis of final target >99% ee, t_{R}=5.11 min, on 4.6×250 mm ChiralPak AD-H, 5 micron column, 2.37 mL/min, 15:85 (MeOH containing 0.5% dimethylethylamine): supercritical CO₂, UV-DAD and MS detection.

### Example 29

### (4-Isopropyl-piperazin-1-yl)-[6-(tetrahydro-pyran-4-yl)-6-aza-spiro[2.5]oct-1-yl]-methanone, single enantiomer

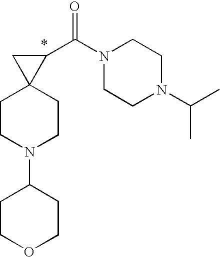 [0427] Note: * designates single enantiomer of unknown stereochemistry.

This compound was prepared according to Example 27 using the remaining enantiomer isolated from Example 28, and commercially available tetrahydro-pyran-4-one. m/z (ES+) M+1=350; HPLC t_{R}=0.17 min; ¹H NMR (500 MHz, CDCl₃) δ ppm 0.71 (dd, J=7.63, 4.58 Hz, 1H) 1.04 (d, J=6.71 Hz, 6H) 1.24 (t, J=4.88 Hz, 1H) 1.33-1.40 (m, 1H) 1.41-1.48 (m, 1H) 1.55-1.67 (m, 4H) 1.67-1.79 (m, 3H) 2.36-2.63 (m, 8H) 2.65-2.75 (m, 2H) 3.37 (t, J=11.60 Hz, 2H) 3.46-3.56 (m, 1H) 3.63 (br. s., 2H) 3.71-3.81 (m, 1H) 4.02 (dd, J=11.29, 3.97 Hz, 2H); Analytical Chiral SFC analysis of final target >99% ee, t_{R}=6.95 min, on 4.6×250 mm ChiralPak AD-H, 5 micron column, 2.37 mL/min, 15:85 (MeOH containing 0.5% dimethylethylamine): supercritical CO₂, UV-DAD and MS detection.

### Example 30

### (4-Cyclobutyl-piperazin-1-yl)-[6-(tetrahydro-thiopyran-3-yl)-6-aza-spiro[2.5]oct-1-yl]-methanone

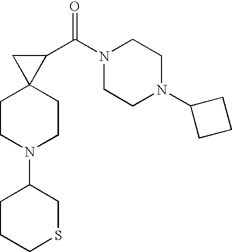

### 30A. 1-(piperazine-1-carbonyl)-6-aza-spiro[2.5]octane-6-carboxylic acid benzyl ester

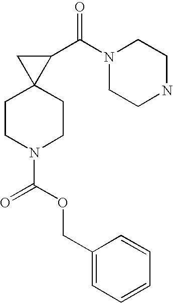

To 1D (6.50 g, 14.21 mmol) in DCM (100 mL) was added TFA (50 mL). After stirring at RT for 1 h, the solvent was removed under reduced pressure and the oil put under high vacuum for 2 h. The oil was partitioned between EtOAc/NaHCO₃ and the organic layer washed 2×NaHCO₃, 2×NaCl, dried over Na₂SO₄, and the solvent removed under reduced pressure to give a yellow gum. This was dissolved in 20 mL DCM, filtered through a 0.45 um filter, and the filtrate was removed of solvent under reduced pressure and put under high vacuum to give 3.305 g 30A as a yellow gum. A second crop of material was obtained by adding solid K₂CO₃ to the aq. layer and extracting with DCM 5×. The combined organic layers were dried over Na₂SO₄ and the solvent removed under reduced pressure and material put under high vacuum to give 1.944 g 30A as a clear gum (103%). m/z (ES+) M+1=358; HPLC t_{R}=4.23 min; ¹H NMR (500 MHz, CDCl₃) δ ppm 0.80 (dd, J=7.63, 4.58 Hz, 1H) 1.31 (t, J=4.58 Hz, 2H) 1.38-1.48 (m, 1H) 1.52-1.69 (m, 3H) 2.41 (br. s., 1H) 2.76-2.92 (m, 3H) 2.95 (br. s., 1H) 3.25-3.33 (m, 1H) 3.33-3.42 (m, 1H) 3.51-3.81 (m, 6H) 5.14 (s, 2H) 7.28-7.40 (m, 5H).

### 30B. 1-(4-Cyclobutyl-piperazine-1-carbonyl)-6-aza-spiro[12.5]octane-6-carboxylic acid benzyl ester

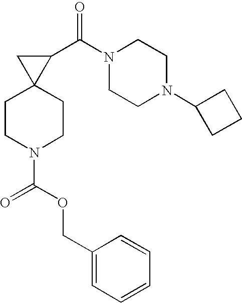

To 30A (3.30 g, 9.23 mmol) was added DCE (50 mL) and cyclobutanone (1.035 mL, 13.85 mmol). The mixture was stirred 25 min and sodium triacetoxyborohydride (2.446 g, 11.54 mmol) was added and the reaction stirred over night. The reaction was quenched with 20 mL 1N NaOH followed by 3 mL 50% NaOH and was stirred for 1 h. Et₂O (50 mL) was added and the aq. layer removed. The organic layer was washed 1×NaHCO₃, 1×NaCl and set aside. The combined aq. layers were washed 2×DCM and the combined organics dried over Na₂SO₄, solvent removed under reduced pressure, and the yellow gum put under high vacuum. The gum was dissolved into Et₂O (25 mL) by heating and the material left to sit at RT. After 4 h at RT the supernate was decanted off and solids washed with a small amount of Et₂O. The supernate was removed of solvent and put under high vacuum to give 1.2 g crude material. The isolated solids were also put under high vacuum. The crude material was chromatographed on 20 g silica eluting with EtOAc until the impurities were removed. The product was eluted by washing with 10% MeOH/EtOAc followed by 15% MeOH/EtOAc. The combined purified fractions were mixed with the solids from the Et₂O step and the solvent removed under reduced pressure. The material was put under high vacuum over night to give 3.29 g 30B as a pale yellow gum (87%). m/z (ES+) M+1=412; HPLC t_{R}=1.78 min; ¹H NMR (500 MHz, CDCl₃) δ ppm 0.78 (dd, J=7.93, 4.88 Hz, 1H) 1.28-1.37 (m, 2H) 1.37-1.47 (m, 1H) 1.52-1.67 (m, 3H) 1.67-1.78 (m, 2H) 1.82-1.93 (m, 2H) 2.00-2.09 (m, 2H) 2.13-2.25 (m, 2H) 2.32-2.39 (m, 1H) 2.39-2.47 (m, 1H) 2.68-2.78 (m, 1H) 3.26-3.34 (m, 1H) 3.35-3.42 (m, 1H) 3.50 (br. s., 1H) 3.62 (br. s., 3H) 3.68-3.85 (m, 2H) 5.14 (s, 2H) 7.28-7.39 (m, 5H).

### 30C. (6-Aza-spiro[2.5]oct-1-yl)-(4-cyclobutyl-piperazin-1-yl)-methanone

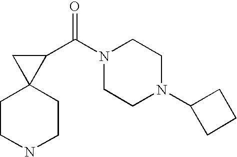

To 30B (from 2 combined batches) (5.18 g, 12.59 mmol) in MeOH (50 mL) was added Pd/C 5% (0.536 g, 0.25 mmol) and a balloon filled with hydrogen fixed atop and the reaction left to stir over night. The catalyst was filtered off, solvent removed under reduced pressure, and white solid put under high vacuum to give 3.416 g 30C as a white solid (98%). m/z (ES+) M+1=278; HPLC t_{R}=0.32 min; ¹H NMR (500 MHz, CDCl₃) δ ppm 0.72 (dd, J=7.63, 4.58 Hz, 1H) 1.25 (t, J=4.88 Hz, 1H) 1.27-1.34 (m, 1H) 1.35-1.42 (m, 1H) 1.52-1.65 (m, 3H) 1.65-1.81 (m, 3H) 1.82-1.95 (m, 2H) 1.99-2.09 (m, 2H) 2.14-2.22 (m, 1H) 2.22-2.31 (m, 1H) 2.32-2.40 (m, 1H) 2.40-2.48 (m, 1H) 2.73 (quip, J=7.93 Hz, 1H) 2.77-2.84 (m, 2H) 2.84-2.97 (m, J=12.05, 12.05, 11.90, 11.60 Hz, 2H) 3.44-3.55 (m, 1H) 3.58-3.72 (m, 2H) 3.73-3.86 (m, 1H).

### 30D. Dihydro-thiopyran-3-one

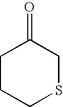

To a −78° C. cooled solution of oxalyl chloride (4.07 mL, 46.53 mmol) in DCM (100 mL) was slowly added via syringe DMSO (6.60 mL, 93.06 mmol) in 20 mL DCM. This solution stirred cold for 15 min and a solution of commercially available tetrahydro-2H-thiopyran-3-ol (5.00 g, 42.30 mmol) in 5 mL DCM was added. The reaction became cloudy and was stirred cold for 15 min. To this mixture was added TEA (29.5 mL, 211.51 mmol) and reaction allowed to warm to RT. After 1 h, the reaction was poured onto a 600 mL filter funnel containing silica gel and eluted with DCM. The semi pure product was removed of solvent under reduced pressure and the resulting material chromatographed on 120 g silica gel column, eluting with DCM. The purified fractions were combined and removed of solvent under reduced pressure. The material was placed under high vacuum 1 h to give 2.211 g 30D as a yellow oil (45%). ¹H NMR (500 MHz, CDCl₃) δ ppm 2.39-2.50 (m, 4H) 2.79 (t, 2H) 3.21 (s, 2H); ¹³C NMR (126 MHz, CDCl₃) 8 ppm 28.62, 33.46, 38.71, 41.91, 203.87.

### 30E. O-Cyclobutyl-piperazin-1-yl)-[6-(tetrahydro-thiopyran-3-yl)-6-aza-spiro[2.5]oct-1-yl]-methanone

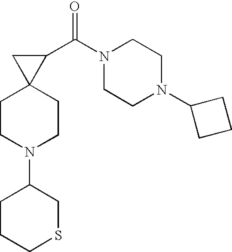

To 30C (200 mg, 0.72 mmol) was added 30D (126 mg, 1.08 mmol), DCE (1.00 mL), and lastly sodium triacetoxyborohydride (229 mg, 1.08 mmol). The reaction was left to stir at RT over night. The reaction was quenched with NaHCO₃ and the aq. was removed. The organic layer was removed of solvent and was purified by reverse phase chromatography on a 30 mm×100 mm, 5 [symbol] Gemini C18 column at pH 10 [NH₄HCO₃] with ACN/H₂O as the mobile phase. Solvent flow was 50 ml/min, and the gradient runs from 5 to 95% ACN over 10 min Fractions were collected by MS detection and were removed of solvent using a Genevac Series II rotary evaporator. The resulting solids were redissolved in ACN/H₂O, and lyophilized to 272 mg 30E as a white solid (74%). m/z (TOF ES+) M+1=378; HPLC t_{R}=0.15 min, HRMS (TOF ES+) m/z calc'd for C₂₁H₃₅N₃OS [M+H]⁺, 378.2574. found, 378.2574. ¹H NMR (500 MHz, DMSO-d₆) δ ppm 0.62 (dd, J=7.93, 3.66 Hz, 1H) 0.96 (t, J=4.27 Hz, 1H) 1.25-1.33 (m, 1H) 1.34-1.47 (m, 4H) 1.55-1.68 (m, 3H) 1.71-1.85 (m, 4H) 1.89-1.99 (m, 2H) 2.03-2.15 (m, 2H) 2.15-2.27 (m, 2H) 2.28-2.46 (m, 4H) 2.51-2.64 (m, 6H) 2.65-2.75 (m, 1H) 3.30-3.40 (m, 1H) 3.49-3.67 (m, 3H).

### Example 31

### (4-Cyclobutyl-piperazin-1-yl)-(6-cyclohexyl-6-aza-spiro[2.5]oct-1-yl)-methanone

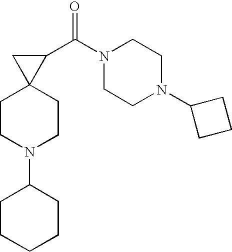

This example was prepared according to Example 30 using 30C and commercially available cyclohexanone. m/z (ES+) M+1=360; HPLC t_{R}=0.36 min; ¹H NMR (500 MHz, DMSO-d₆) δ ppm 0.62 (dd, J=7.63, 3.97 Hz, 1H) 0.96 (t, J=4.58 Hz, 1H) 1.05 (d, J=8.54 Hz, 1H) 1.11-1.25 (m, 4H) 1.26-1.35 (m, 1H) 1.35-1.47 (m, 3H) 1.50-1.85 (m, 10H) 1.89-2.00 (m, 2H) 2.01-2.10 (m, 1H) 2.14-2.28 (m, 3H) 2.29-2.46 (m, 3H) 2.51-2.56 (m, 2H) 2.65-2.75 (m, 1H) 3.29-3.38 (m, 1H) 3.51-3.64 (m, 3H).

### Example 32

### (4-Cyclobutyl-piperazin-1-yl)-[6-(tetrahydro-pyran-3-yl)-6-aza-spiro[2.5]oct-1-yl]-methanone

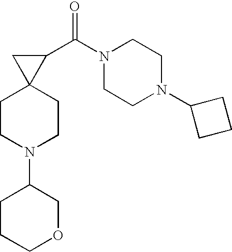

This example was prepared according to Example 30 using 30C and dihydro-pyran-3-one, which was prepared according to the procedures described by Brown, H. C., et al. J. Org. Chem. 1985; 50(10), 1577-1582 and in U.S. Pat. No. 4,410,354. m/z (TOF ES+) M+1=362; HPLC t_{R}=0.12 min, HRMS (TOF ES+) m/z calc'd for C₂₁H₃₅N₃O₂ [M+H]⁺, 362.2802. found, 362.2794. ¹H NMR (500 MHz, DMSO-d₆) δ ppm 0.62 (dd, J=7.93, 3.66 Hz, 1H) 0.96 (t, J=4.58 Hz, 1H) 1.24-1.33 (m, 1H) 1.34-1.53 (m, 5H) 1.55-1.69 (m, 3H) 1.70-1.83 (m, 3H) 1.84-1.90 (m, 1H) 1.91-2.01 (m, 2H) 2.01-2.13 (m, 1H) 2.14-2.28 (m, 2H) 2.29-2.41 (m, 3H) 2.40-2.47 (m, 1H) 2.55 (br. s., 2H) 2.65-2.78 (m, 1H) 3.09-3.21 (m, 2H) 3.29-3.40 (m, 1H) 3.47-3.64 (m, 3H) 3.71 (d, J=11.60 Hz, 1H) 3.78-3.89 (m, 1H).

### Example 33

### (4-Cyclobutyl-piperazin-1-yl)-[6-(tetrahydro-furan-3-yl)-6-aza-spiro[2.5]oct-1-yl]-methanone

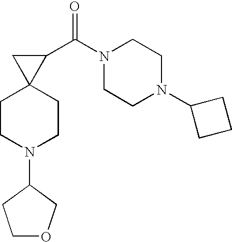

This example was prepared according to Example 30 using 30C and commercially available dihydrofuran-3(2H)-one. m/z (TOF ES+) M+1=348; HPLC t_{R}=0.11 min, HRMS (TOF ES+) m/z calc'd for C₂₀H₃₃N₃O₂ [M+H]⁺, 348.2646. found, 348.2644. ¹H NMR (500 MHz, DMSO-d₆) δ ppm 0.64 (dd, J=7.32, 3.66 Hz, 1H) 0.97 (t, J=4.58 Hz, 1H) 1.27-1.38 (m, 1H) 1.38-1.52 (m, 3H) 1.57-1.74 (m, 3H) 1.74-1.85 (m, 3H) 1.91-2.01 (m, 3H) 2.04-2.12 (m, 1H) 2.12-2.30 (m, 3H) 2.36 (br. s., 3H) 2.42-2.48 (m, 1H) 2.65-2.74 (m, 1H) 2.82-2.92 (m, 1H) 3.30-3.39 (m, 1H) 3.40-3.47 (m, 1H) 3.49-3.57 (m, 1H) 3.57-3.67 (m, 3H) 3.75 (t, J=7.93 Hz, 2H).

### Example 34

### (4-Cyclobutyl-piperazin-1-yl)-[6-(2-morpholin-4-yl-ethyl)-6-aza-spiro[2.5]oct-1-yl]-methanone

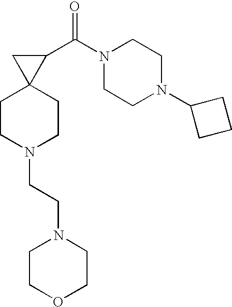

To 30C (100 mg, 0.36 mmol) was added 4-(2-chloroethyl)morpholine hydrochloride (73.8 mg, 0.40 mmol), THF (1.0 mL) and lastly TEA (0.126 mL, 0.90 mmol). The reaction left to stir at RT for 1 h then at 50° C. for 2 h after which TBAI (13.32 mg, 0.04 mmol) was added and the reaction heated at 50° C. over night. To this mixture was added DMF (1 mL) and the reaction heated to 100° C. in the microwave for 15 min. The reaction was cooled to RT and the resulting crystals were removed by filtration and the filtrate removed of solvent under reduced pressure and put under high vacuum. The filtrate was purified by reverse phase chromatography on a 30 mm×100 mm, 5 [symbol] Gemini C18 column at pH 10 [NH₄HCO₃] with ACN/H₂O as the mobile phase. Solvent flow was 50 ml/min, and the gradient runs from 5 to 95% ACN over 10 min Fractions were collected by MS detection and removed of solvent using a Genevac Series II rotary evaporator. The resulting solids were redissolved in ACN/H₂O, and lyophilized to give 9.2 mg of title compound as a white solid (6.5%). m/z (TOF ES+) M+1=391; HPLC t_{R}=0.11 min, HRMS (TOF ES+) m/z calc'd for C₂₂H₃₈N₄O₂ [M+H]⁺, 391.3068. found, 391.3066. ¹H NMR (500 MHz, DMSO-d₆) δ ppm 0.63 (dd, J=7.93, 3.66 Hz, 1H) 0.96 (t, J=4.58 Hz, 1H) 1.27-1.36 (m, 1H) 1.37-1.49 (m, 3H) 1.57-1.68 (m, 2H) 1.72-1.85 (m, 3H) 1.90-2.01 (m, 2H) 2.03-2.12 (m, 1H) 2.15-2.28 (m, 3H) 2.30-2.45 (m, 10H) 2.65-2.75 (m, 1H) 3.22-3.39 (m, 4H, under water peak) 3.49-3.57 (m, 4H) 3.58-3.64 (m, 2H).

### Example 35

### (4-Cyclohexyl-piperazin-1-yl)-(6-pyridin-4-yl-6-aza-spiro[2.5]oct-1-yl)-methanone

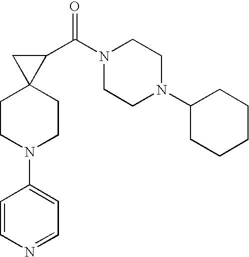

To 16F (100 mg, 0.33 mmol) was added 4-chloropyridine hydrochloride (49.1 mg, 0.33 mmol), K₂CO₃ (113 mg, 0.82 mmol) and DMSO (2 mL). The reaction was sealed and placed in a 90° C. bath over night. To the reaction was added EtOAc/NaCl and the organic layer washed 2×NaCl, aq. washed 2×EtOAc, and the combined organic layers dried over Na₂SO₄ and the solvent removed under reduced pressure. The material was purified on 12 g silica gel eluting with DCM to 10% (7N NH₃/MeOH)/DCM over 11 min, 30 mL/min. The combined purified fractions were removed of solvent under reduced pressure and put under high vacuum 50° C. over night to give 32.8 mg of title compound as a white solid (26%). m/z (ES+) M+1=383; HPLC t_{R}=0.65 min; m/z (TOF ES+) M+1=383; HPLC t_{R}=0.27 min, HRMS (TOF ES+) m/z calc'd for C₂₃H₃₄N₄O [M+H]⁺, 383.2805. found, 383.2806. ¹H NMR (500 MHz, CDCl₃) δ ppm 0.83 (dd, J=7.93, 4.27 Hz, 1H) 1.03-1.29 (m, 4H) 1.35 (t, J=4.58 Hz, 1H) 1.42-1.50 (m, 1H) 1.52-1.65 (m, 3H) 1.66-1.75 (m, 3H) 1.75-1.87 (m, 4H) 2.21-2.31 (m, 1H) 2.40-2.52 (m, 2H) 2.53-2.67 (m, 2H) 3.21-3.29 (m, 1H) 3.30-3.38 (m, 1H) 3.39-3.46 (m, 1H) 3.47-3.56 (m, 2H) 3.60 (br. s., 2H) 3.69-3.79 (m, 1H) 6.66 (d, J=6.10 Hz, 2H) 8.25 (d, J=6.10 Hz, 2H).

### Example 36

### [1-(4-Cyclobutyl-piperazine-1-carbonyl)-6-aza-spiro[2.5]oct-6-yl]-acetic acid tert-butyl ester

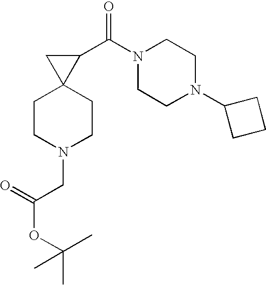

To 30C (200 mg, 0.72 mmol) in THF (2.0 mL) was added TEA (0.111 mL, 0.79 mmol) and tert-butyl 2-bromoacetate (0.112 mL, 0.76 mmol). The reaction was stirred at RT for 2 h. The solvent was removed under reduced pressure and material put under high vacuum over night. The material was chromatographed on 40 g silica 0-20% MeOH/DCM. The combined purified fractions were removed of solvent under reduced pressure to give 248 mg of title compound as a white solid (88%). m/z (ES+) M+1=392; HPLC t_{R}=2.07 min (15 min run); ¹H NMR (500 MHz, CDCl₃) δ ppm 0.71 (dd, J=7.63, 4.58 Hz, 1H) 1.24 (t, J=4.88 Hz, 1H) 1.34 (br. s., 1H) 1.37-1.49 (m, 9H) 1.53-1.61 (m, 2H) 1.63-1.82 (m, 4H) 1.82-1.95 (m, 2H) 1.97-2.09 (m, 2H) 2.10-2.19 (m, 1H) 2.20-2.30 (m, 1H) 2.32-2.47 (m, 3H) 2.47-2.56 (m, 1H) 2.57-2.68 (m, 1H) 2.67-2.79 (m, 2H) 3.12 (s, 2H) 3.40-3.50 (m, 1H) 3.55-3.64 (m, 1H) 3.64-3.72 (m, 1H) 3.76-3.87 (m, 1H).

### Example 37

### 2-[1-(4-Cyclobutyl-piperazine-1-carbonyl)-6-aza-spiro[2.5]oct-6-yl]-N-methyl-acetamide

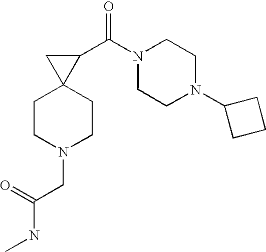

### 37A. [1-(4-Cyclobutyl-piperazine-1-carbonyl)-6-aza-spiro[2.5]oct-6-yl]-acetic acid

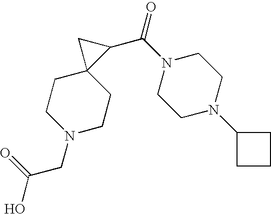

To Example 36 (231 mg, 0.59 mmol) in acetic acid (5 mL) was bubbled in HCl gas (excess) and the reaction was left to stir at RT for 1 h. The solvent was removed under reduced pressure and residue put under high vacuum 50° C. to give 247 mg 37A (125%). m/z (TOF ES+) M+1=333; HPLC t_{R}=0.12 min

### 37B. 2-[1-(4-Cyclobutyl-piperazine-1-carbonyl)-6-aza-spiro[2.5]oct-6-yl]-N-methyl-acetamide

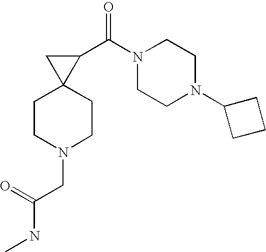

To 37A (112 mg, 0.27 mmol) was added SOCl₂ (2.00 mL, 27.40 mmol) and the reaction was vigorously stirred at RT. After 1.5 h the reaction was removed of solvent and yellow foam put under high vacuum for 1 h. To this was added methylamine 2.0M in THF (5.00 mL, 10.00 mmol) and the reaction was stirred for 10 min. The reaction was removed of solvent under reduced pressure and put under high vacuum over night. The crude was purified by reverse phase chromatography on a 30 mm×100 mm, 5μ Gemini C18 column at pH 10 [NH₄HCO₃] with ACN/H₂O as the mobile phase. Solvent flow was 50 ml/min, and the gradient runs from 5 to 95% ACN over 10 min Fractions were collected by MS detection and were removed of solvent using a Genevac Series II rotary evaporator. The resulting solids were redissolved in ACN/H₂O, and lyophilized to give 51 mg 37B as a white solid (53%). m/z (TOF ES+) M+1=349; HPLC t_{R}=0.11 min, HRMS (TOF ES+) m/z calc'd for C₁₉H₃₂N₄O₂ [M+H]⁺, 349.2598. found, 349.2596. ¹H NMR (500 MHz, DMSO-d₆) δ ppm 0.65 (dd, J=7.32, 3.66 Hz, 1H) 0.92-1.01 (m, 1H) 1.37 (br. s., 1H) 1.47 (d, J=3.66 Hz, 3H) 1.57-1.70 (m, 2H) 1.72-1.86 (m, 3H) 1.90-2.02 (m, 2H) 2.08 (br. s., 1H) 2.15-2.30 (m, 3H) 2.35 (br. s., 2H) 2.44 (br. s., 2H) 2.61 (d, J=4.88 Hz, 3H) 2.65-2.76 (m, J=7.93, 7.63, 7.48, 7.48 Hz, 1H) 2.87 (s, 2H) 3.31-3.40 (m, 1H) 3.47-3.68 (m, 3H) 7.61 (br. s., 1H).

### Example 38

### (4-Cyclobutyl-piperazin-1-yl)-[6-(4-methanesulfonyl-phenyl)-6-aza-spiro[2.5]oct-1-yl]-methanone

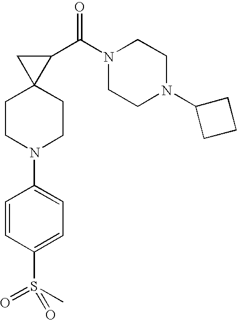

To 4-fluorothioanisole (2.00 mL, 16.41 mmol) in acetone (20 mL) at 0° C. was added H₂O₂ 30% (2.108 mL, 24.62 mmol) and reaction stirred cold until ice melted. After the reaction was stirred over night additional H₂O₂ 30% (2.108 mL, 24.62 mmol) was added to the reaction at RT and the reaction stirred for 24 h. The solvent was removed under reduced pressure using RT bath and material placed under high vacuum, (2.54 g, 98%). ¹H NMR (300 MHz, CDCl₃) δ ppm 3.06 (s, 3H) 7.14-7.34 (m, 2H, under CHCl₃) 7.88-8.10 (m, 2H). The crude material was combined with 28C according to Example 33 to give title compound as a white solid. m/z (TOF ES+) M+1=432; HPLC t_{R}=5.74 min; NMR (500 MHz, DMSO-d₆) δ ppm 0.77 (dd, J=7.63, 3.97 Hz, 1H) 1.07 (t, J=4.58 Hz, 1H) 1.39-1.48 (m, 1H) 1.48-1.67 (m, 5H) 1.70-1.84 (m, 2H) 1.87-1.98 (m, 3H) 2.08-2.24 (m, 3H) 2.25-2.35 (m, 1H) 2.66 (quip, J=7.63 Hz, 1H) 3.08 (s, 3H) 3.23-3.32 (m, 1H, under H₂O) 3.32-3.43 (m, 2H) 3.43-3.47 (m, 2H) 3.47-3.60 (m, 2H) 3.60-3.69 (m, 1H) 7.07 (d, J=9.16 Hz, 2H) 7.65 (d, J=9.16 Hz, 2H).

### Example 39

### (4-Cyclobutyl-piperazin-1-yl)-[6-(4-methoxy-phenyl)-6-aza-spiro[2.5]oct-1-yl]-methanone

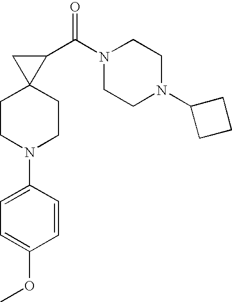

To 30C (100 mg, 0.36 mmol) was added trans-dichlorobis(tri-o-tolyl phosphine)palladium(II) (28.3 mg, 0.04 mmol), toluene (1.5 mL), 1-bromo-4-methoxybenzene (0.068 mL, 0.54 mmol), and lithium bis(trimethylsilyl)amide (1M in toluene) (0.433 mL, 0.43 mmol). The reaction was heated 100° C. for 2 h cooled to RT and stirred over night. The solvent was removed under reduced pressure and the crude material placed under high vacuum. The crude was purified by reverse phase chromatography on a 30 mm×100 mm, 5μ Gemini C18 column at pH 10 [NH₄HCO₃] with ACN/H₂O as the mobile phase. Solvent flow was 50 ml/min, and the gradient runs from 5 to 95% ACN over 10 min Fractions were collected by MS detection and were removed of solvent using a Genevac Series II rotary evaporator. The resulting solids were redissolved in ACN/H₂O, and lyophilized to give 74.6 mg title compound as a white solid (54%). m/z (TOF ES+) M+1=384; HPLC t_{R}=1.73 min; ¹H NMR (500 MHz, DMSO-d₆) δ ppm 0.72 (dd, J=7.63, 3.97 Hz, 1H) 1.05 (t, J=4.58 Hz, 1H) 1.42-1.52 (m, 1H) 1.53-1.68 (m, 5H) 1.71-1.83 (m, 2H) 1.84-1.89 (m, 1H) 1.90-1.99 (m, 2H) 2.05-2.13 (m, 1H) 2.14-2.27 (m, 2H) 2.28-2.38 (m, 1H) 2.61-2.72 (m, 1H) 2.83-2.93 (m, 1H) 2.92-3.00 (m, 1H) 3.01-3.12 (m, 2H) 3.33-3.42 (m, 1H) 3.49-3.65 (m, 3H) 3.68 (s, 3H) 6.80 (d, J=9.16 Hz, 2H) 6.89 (d, J=9.16 Hz, 2H).

### Example 40

### 4-[1-(4-Cyclobutyl-piperazine-1-carbonyl)-6-aza-spiro[2.5]oct-6-yl]-benzoic acid methyl ester

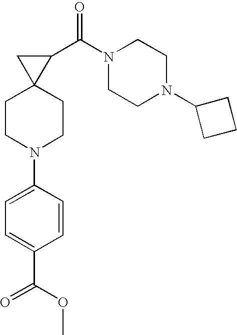

This example was prepared according to Example 39 using 30C and commercially available methyl 4-bromobenzoate. m/z (TOF ES+) M+1=412; HPLC t_{R}=7.84 min; ¹H NMR (500 MHz, DMSO-d₆) δ ppm 0.76 (dd, J=7.93, 4.27 Hz, 1H) 1.07 (t, J=4.58 Hz, 0H) 1.39-1.48 (m, 0H) 1.48-1.67 (m, 5H) 1.69-1.82 (m, 2H) 1.87-1.99 (m, 3H) 2.05-2.17 (m, 2H) 2.17-2.25 (m, 1H) 2.26-2.34 (m, 1H) 2.65 (quip, J=7.63 Hz, 1H) 3.22-3.34 (m, 3H, under H₂O) 3.34-3.47 (m, 4H) 3.48-3.67 (m, 3H) 3.77 (s, 3H) 6.97 (d, J=9.16 Hz, 2H) 7.76 (d, J=9.16 Hz, 2H).

### Example 41

### (4-Cyclobutyl-piperazin-1-yl)-[6-(6-methoxy-pyridin-2-yl)-6-aza-spiro[2.5]oct-1-yl]-methanone

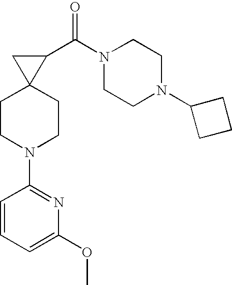

This example was prepared according to Example 35 using 30C and commercially available 2-chloro-6-methoxypyridine. m/z (TOF ES+) M+1=385; HPLC t_{R}=6.64 min; ¹H NMR (500 MHz, DMSO-d₆) δ ppm 0.75 (dd, J=7.63, 3.97 Hz, 1H) 1.07 (t, J=4.58 Hz, 1H) 1.36-1.43 (m, 1H) 1.44-1.68 (m, 5H) 1.70-1.82 (m, 2H) 1.86-1.99 (m, 3H) 2.04-2.26 (m, 3H) 2.31 (br. s., 1H) 2.66 (t, J=7.63 Hz, 1H) 3.34-3.42 (m, 2H) 3.43-3.68 (m, 7H) 3.75 (s, 3H) 5.99 (d, J=7.32 Hz, 1H) 6.31 (d, J=7.93 Hz, 1H).

### Example 42

### [6-(6-Chloro-pyridin-2-yl)-6-aza-spiro[2.5]oct-1-yl]-(4-cyclobutyl-piperazin-1-yl)-methanone

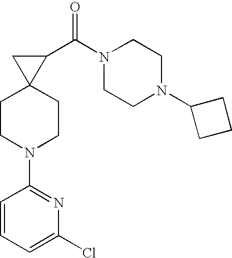

This example was isolated from the preparation of Example 41. m/z (TOF ES+) M+1=389; HPLC t_{R}=8.35 min; ¹H NMR not taken.

### Example 43

### (4-Cyclohexyl-piperazin-1-yl)-[6-(2-methoxy-ethyl)-6-aza-spiro[2.5]oct-1-yl]-methanone

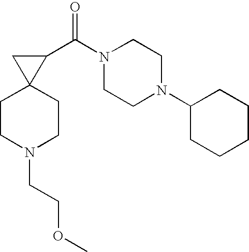

To 30F (100 mg, 0.33 mmol), was added K₂CO₃ (90 mg, 0.65 mmol), LiI (8.76 mg, 0.07 mmol), ACN (2.0 mL), and 1-bromo-2-methoxyethane (0.031 mL, 0.33 mmol). After stirring the mixture at RT over night, EtOAc was added, the salts filtered off, and filtrate was removed of solvent under reduced pressure. Material was purified on 12 g silica gel eluting with DCM to 20% (7N NH₃/MeOH)/DCM over 11 min, 30 mL/min. The semi-pure product was removed of solvent and put under high vacuum. The sample was dissolved in 1.25M HCl in MeOH then blown down and put under high vacuum 50° C. The solids were triturated with Et₂O filtered and put under high vacuum. The semi pure material further was purified by reverse phase chromatography on a 30 mm×100 mm, 5μ Gemini C18 column at pH 10 [NH₄HCO₃] with ACN/H₂O as the mobile phase. Solvent flow was 50 ml/min, and the gradient runs from 5 to 95% ACN over 10 min Fractions were collected by MS detection and removed of solvent using a Genevac Series II rotary evaporator. The resulting solids were redissolved in ACN/H₂O, and lyophilized to give 17.1 mg title compound as a white solid (14%). m/z (TOF ES+) M+1=364; HPLC t_{R}=0.20 min, HRMS (TOF ES+) m/z calc'd for C₂₁H₃₇N₃O₂ [M+H]⁺, 364.2959. found, 364.2955. ¹H NMR (500 MHz, DMSO-d₆) δ ppm 0.63 (dd, J=7.93, 3.66 Hz, 1H) 0.96 (t, J=4.27 Hz, 1H) 1.01-1.12 (m, 1H) 1.12-1.26 (m, 5H) 1.27-1.37 (m, 1H) 1.37-1.48 (m, 3H) 1.57 (d, J=11.60 Hz, 1H) 1.67-1.80 (m, 5H) 2.25 (br. s., 2H) 2.29-2.39 (m, 2H) 2.40-2.48 (m, 6H) 2.54-2.67 (m, 1H) 3.22 (s, 3H) 3.41 (t, J=5.80 Hz, 2H) 3.50-3.66 (m, 3H).

### Example 44

### (4-Cyclobutyl-piperazin-1-yl)-(6-pyridin-2-yl-6-aza-spiro[2.5]oct-1-yl)-methanone

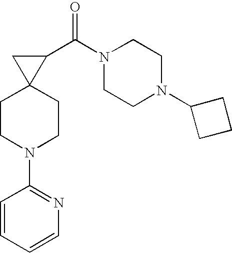

This example was prepared according to Example 35 using 30C and commercially available 2-chloropyridine. m/z (TOF ES+) M+1=355; HPLC t_{R}=0.18 min, HRMS (TOF ES+) m/z calc'd for C₂₁H₃₀N₄O [M+H]⁺, 355.2492. found, 355.2493. ¹H NMR (500 MHz, DMSO-d₆) δ ppm 0.75 (dd, J=7.78, 3.81 Hz, 1H) 1.06 (d, J=4.58 Hz, 1H) 1.38 (ddd, J=13.20, 7.10, 3.81 Hz, 1H) 1.43-1.68 (m, 5H) 1.69-1.83 (m, 2H) 1.86-1.98 (m, 3H) 2.04-2.17 (m, 2H) 2.18-2.25 (m, 1H) 2.27-2.34 (m, 1H) 2.66 (quip, J=7.55 Hz, 1H) 3.34-3.67 (m, 8H) 6.58 (dd, J=7.02, 5.19 Hz, 1H) 6.81 (d, J=8.55 Hz, 1H) 7.49 (ddd, J=8.70, 7.02, 1.98 Hz, 1H) 8.08 (dd, J=4.88, 1.22 Hz, 1H).

### Example 45

### (4-Cyclobutyl-piperazin-1-yl)-(6-p-tolyl-6-aza-spiro[2.5]oct-1-yl)-methanone

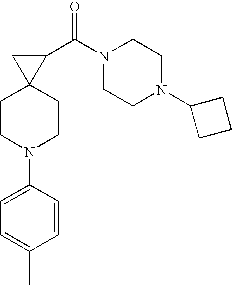

This example was prepared according to Example 39 using 30C and commercially available 4-bromotoluene. m/z (TOF ES+) M+1=368; HPLC t_{R}=3.03 min; ¹H NMR (500 MHz, DMSO-d₆) δ ppm 0.73 (dd, J=7.78, 3.81 Hz, 1H) 1.04 (t, 1H) 1.41-1.50 (m, 1H) 1.52-1.66 (m, 5H) 1.70-1.82 (m, 2H) 1.88 (dd, J=7.93, 5.19 Hz, 1H) 1.90-1.98 (m, 2H) 2.04-2.12 (m, 1H) 2.13-2.26 (m, 5H) 2.27-2.35 (m, 1H) 2.61-2.72 (m, 1H) 2.91-2.99 (m, 1H) 3.00-3.08 (m, 1H) 3.08-3.20 (m, 2H) 3.33-3.41 (m, 1H) 3.48-3.69 (m, 3H) 6.83 (d, J=8.54 Hz, 2H) 7.00 (d, 2H).

### Example 46

### (4-Cyclohexyl-piperazin-1-yl)-[6-(5-trifluoromethyl-pyridin-2-yl)-6-aza-spiro[2.5]oct-1-yl]-methanone

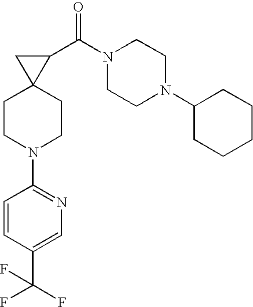

This example was prepared according to Example 35 using 16F and commercially available 2-chloro-5-(trifluoromethyl)pyridine. m/z (ES+) M+1=451; HPLC t_{R}=4.88 min; m/z (TOF ES+) M+1=451; HPLC t_{R}=1.02 min, HRMS (TOF ES+) m/z calc'd for C₂₄H₃₃F₃N₄O [M+H]⁺, 451.2679. found, 451.2681. ¹H NMR (500 MHz, CDCl₃) δ ppm 0.84 (dd, J=7.93, 4.58 Hz, 1H) 1.02-1.29 (m, 4H) 1.36 (t, J=4.73 Hz, 1H) 1.40-1.47 (m, 1H) 1.51-1.58 (m, 2H) 1.59-1.73 (m, 4H) 1.74-1.88 (m, 4H) 2.26 (t, J=10.68 Hz, 1H) 2.41-2.52 (m, 2H) 2.53-2.68 (m, 2H) 3.41-3.48 (m, 1H) 3.49-3.56 (m, 1H) 3.57-3.67 (m, 3H) 3.69-3.81 (m, 2H) 3.90-3.99 (m, 1H) 6.64 (d, J=9.16 Hz, 1H) 7.60 (dd, J=9.16, 2.44 Hz, 1H) 8.38 (s, 1H).

### Example 47

### (6-Benzo[1,3]dioxol-5-yl-6-aza-spiro[2.5]oct-1-yl)-(4-cyclohexyl-piperazin-1-yl)-methanone

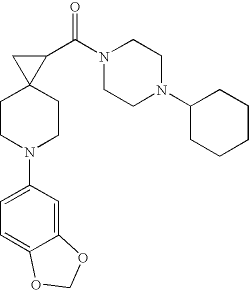

This example was prepared according to Example 39 using 16F and commercially available 5-bromobenzo[d][1,3]dioxole. m/z (ES+) M+1=426; HPLC t_{R}=1.29 min; m/z (TOF ES+) M+1=426; HPLC t_{R}=0.69 min, HRMS (TOF ES+) m/z calc'd for C₂₅H₃₅N₃O₃ [M+H]⁺, 426.2751. found, 426.2750. ¹H NMR (500 MHz, CDCl₃) δ ppm 0.78 (dd, J=7.93, 4.58 Hz, 1H) 1.03-1.29 (m, 5H) 1.31 (t, J=4.73 Hz, 1H) 1.48-1.52 (m, 1H) 1.55-1.68 (m, 3H) 1.72-1.85 (m, 6H) 2.22-2.31 (m, 1H) 2.41-2.67 (m, 4H) 2.92-2.99 (m, 1H) 3.01-3.09 (m, 2H) 3.11-3.20 (m, 1H) 3.46-3.54 (m, 1H) 3.58-3.69 (m, 2H) 3.72-3.81 (m, 1H) 5.89 (s, 2H) 6.38 (dd, J=8.39, 2.29 Hz, 1H) 6.57 (d, J=2.14 Hz, 1H) 6.70 (d, J=8.54 Hz, 1H).

### Example 48

### (4-(pyridin-3-yl)piperazin-1-yl)(6-(tetrahydro-2H-pyran-4-yl)-6-azaspiro[2.5]octan-1-yl)methanone

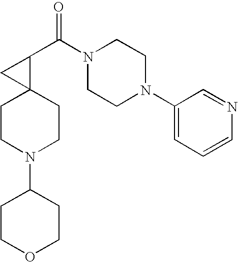

To a solution of 8C (105.3 mg, 0.44 mmol) in DMF (5 mL) was added DIPEA (0.231 mL, 1.32 mmol). The solution was stirred 5 min then 2-(1H-benzo[d][1,2,3]triazol-1-yl)-1,1,3,3-tetramethylisouronium hexafluorophosphate(V) (167 mg, 0.44 mmol), which is commercially available from common vendors, such as, for example, Aldrich was added and the mixture was stirred for another 10 min (solution became brownish orange). 1-(pyridin-3-yl)piperazine, 2HCl (104 mg, 0.44 mmol), which is commercially available from common vendors, such as, for example, Aldrich was added and the reaction mixture stirred over the weekend. The reaction mixture was concentrated under reduced pressure to give 550 mg of crude product that was subsequently purified by high pH HPLC MS (20-40% ACN in H₂O and lyophylised to obtain 88 mg (52%) of title compound (a white solid) as a free base. HRMS[M+H]⁺ calc.=385.25980. obs.=385.26017. HPLC t_{R}=1.06 min ¹H NMR (400 MHz, CDCl₃) δ ppm 0.76 (dd, J=7.81, 4.69 Hz, 1H) 1.28 (t, J=4.69 Hz, 1H) 1.36 (br. s, 1H) 1.45 (br. s, 1H) 1.54-1.70 (m, 4H) 1.77 (br. s, 3H) 2.39-2.57 (m, 3H) 2.59 (br. s, 1H) 2.72 (br. s, 1H) 3.06-3.32 (m, 4H) 3.36 (td, J=11.82, 1.76 Hz, 2H) 3.66-3.76 (m, 1H) 3.80 (br. s, 2H) 3.85-3.95 (m, 1H) 4.02 (dd, J=11.13, 4.10 Hz, 2H) 7.19 (dd, J=2.73, 1.95 Hz, 2H) 8.15 (t, J=2.93 Hz, 1H) 8.32 (t, J=1.76 Hz, 1H).

### Example 49

### (4-(2-methylpyridin-4-yl)piperazin-1-yl)(6-(tetrahydro-2H-pyran-4-yl)-6-azaspiro[2.5]octan-1-yl)methanone

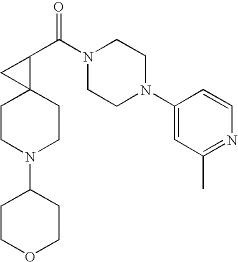

This example was prepared according to Example 48 with 8C (105.3 mg, 0.44 mmol) and 1-(2-methylpyridin-4-yl)piperazine (78 mg, 0.44 mmol), which is commercially available from common vendors, such as, for example, Aldrich. The reaction mixture was stirred overnight and then concentrated under reduced pressure to give 392 mg of crude product that was subsequently purified by high pH HPLC MS (20-40% ACN in H₂O) and lyophylised to obtain 114 mg (65.0%) title compound (white solid) as a free base. HRMS[M+H]⁺ calc.=399.27545. obs.=399.27614. HPLC t_{R}=0.39 min ¹H NMR (400 MHz, CD₃OD) δ ppm 0.83 (dd, J=7.81, 4.30 Hz, 1H) 1.18 (t, J=4.88 Hz, 1H) 1.41-1.69 (m, 6H) 1.81 (br. s, 2H) 1.90 (dd, J=7.81, 5.08 Hz, 1H) 2.41 (s, 3H) 2.47-2.59 (m, 3H) 2.64 (br. s, 1H) 2.75 (br. s, 1H) 3.34-3.43 (m, 3H) 3.43-3.53 (m, 2H) 3.54-3.63 (m, 1H) 3.65-3.74 (m, 1H) 3.75-3.85 (m, 1H) 3.85-3.93 (m, 2H) 3.96 (dd, J=11.33, 4.30 Hz, 2H) 6.72 (dd, J=6.25, 2.73 Hz, 1H) 6.76 (d, J=2.73 Hz, 1H) 8.03 (d, J=6.25 Hz, 1H).

### Example 50

### (4-(3-methylpyridin-4-yl)piperazin-1-yl)(6-(tetrahydro-2H-pyran-4-yl)-6-azaspiro[2.5]octan-1-yl)methanone

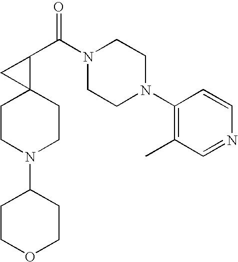

This example was prepared according to Example 48 with 8C (60 mg, 0.25 mmol) and 1-(3-methylpyridin-4-yl)piperazine (44.4 mg, 0.25 mmol), which is commercially available from common vendors, such as, for example, Aldrich. The reaction mixture was stirred overnight and then concentrated under reduced pressure to give 230 mg of crude product that was purified by high pH HPLC MS (20-40% ACN in H₂O) and lyophylised to obtain 61.9 mg (61.9%) title compound (white solid) as a free base. HRMS[M+H]⁺ calc.=399.27545. obs.=399.27601. HPLC t_{R}=0.40 min ¹H NMR (400 MHz, CDCl₃) 8 ppm 0.77 (dd, J=8.01, 4.49 Hz, 1H) 1.29 (t, J=4.69 Hz, 1H) 1.36 (br. s, 1H) 1.45 (br. s, 1H) 1.57-1.87 (m, 7H) 2.29 (s, 3H) 2.40-2.58 (m, 3H) 2.66 (br. s, 1H) 2.75 (br. s, 1H) 2.85-3.15 (m, 4H) 3.38 (td, J=11.82, 1.76 Hz, 2H) 3.68 (ddd, J=12.89, 7.62, 2.93 Hz, 1H) 3.80 (t, J=4.88 Hz, 2H) 3.92 (ddd, J=12.99, 5.96, 2.93 Hz, 1H) 4.04 (dd, J=11.33, 3.91 Hz, 2H) 6.77 (d, J=5.47 Hz, 1H) 8.30-8.37 (m, 2H).

1. A compound of formula I, enantiomers thereof, pharmaceutically acceptable salts thereof, or mixtures thereof: 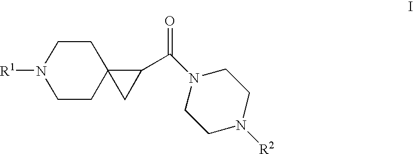
wherein:
R¹ is aryl, heteroaryl, arylalkyl, heteroarylalkyl, cycloalkyl, heterocycloalkylalkyl, alkyl, alkoxyalkyl, hydroxyalkyl, aminoalkyl, aminocarbonylalkyl, heterocycle, arylalkenyl, or heterocycloalkyl; wherein R¹ is optionally substituted with at least one substituent selected independently from alkyl, cyano, sulfinyl, haloalkyl, amide, alkoxy, halogen, arylalkoxy, alkylcarbonyl, carboxy (—C(═O)OH), hydroxy (—OH), amino, alkoxycarbonyl, and alkylsulfonyl; and
R² is aryl, heteroaryl, cycloalkyl, alkyl, heterocycloalkyl, or cycloalkylalkyl; wherein R² is optionally substituted with at least one substituent selected independently from C₁-C₆alkyl, alkoxy, and cycloalkyl;
with the provisos that:
1) when R² is a cyclohexyl, R¹ is not a substituted or unsubstituted phenyl;
2) when R² is aryl, R² is not unsubstituted phenyl; and
3) when R¹ is tetrahydropyran-4-yl and R² is a substituted phenyl, the substituted phenyl is not ortho- or meta-substituted with methyl or meta-substituted with methoxy.

2. A compound according to claim 1, enantiomers thereof, pharmaceutically acceptable salts thereof, or mixtures thereof, wherein R¹ is aryl, heteroaryl, arylalkyl, cycloalkyl, heterocycloalkylalkyl, alkyl, arylalkenyl, heterocycle, or heterocycloalkyl.

3. A compound according to claim 1, enantiomers thereof, pharmaceutically acceptable salts thereof, or mixtures thereof, wherein R¹ is C₆-C₁₄aryl, C₆-C₁₄heteroaryl, (C₆-C₁₄aryl)-(C₁-C₆alkyl), C₃-C₈cycloalkyl, (C₃-C₈heterocycloalkyl)-(C₁-C₆alkyl), C₁-C₆alkyl, (C₆-C₁₄aryl)-(C₁-C₆alkenyl), or C₃-C₈heterocycloalkyl.

4. A compound according to claim 1, enantiomers thereof, pharmaceutically acceptable salts thereof, or mixtures thereof, wherein R¹ is substituted with at least one substituent selected independently from alkyl, haloalkyl, amide, alkoxy, halogen, arylalkoxy, alkoxycarbonyl, and alkylsulfonyl.

5. A compound according to claim 1, enantiomers thereof, pharmaceutically acceptable salts thereof, or mixtures thereof, wherein R¹ is substituted with at least one substituent selected independently from C₁-C₆alkyl, haloC₁-C₆alkyl, amide, C₁-C₆alkoxy, halogen, (C₆-C₁₄aryl)-(C₁-C₆alkoxy), C₁-C₆alkoxycarbonyl, and C₁-C₆alkylsulfonyl.

6. A compound according to claim 1, enantiomers thereof, pharmaceutically acceptable salts thereof, or mixtures thereof, wherein R² is C₆-C₁₄aryl, C₆-C₁₄heteroaryl, C₃-C₈cycloalkyl, C₁-C₆alkyl, C₃-C₈heterocycloalkyl, or (C₃-C₈cycloalkyl)-(C₁-C₆alkyl).

7. A compound according to claim 1, enantiomers thereof, pharmaceutically acceptable salts thereof, or mixtures thereof, wherein R² is substituted with at least one alkoxy.

8. A compound according to claim 1, enantiomers thereof, pharmaceutically acceptable salts thereof, or mixtures thereof, wherein R¹ is piperidinyl, tetrahydropyranyl, phenylethyl, butyl, phenylallyl, cyclohexyl, tetrahydro-2H-thiopyranyl, morpholinylethyl, phenylmethyl, tetrahydrofuranyl, pyridinyl, methyl, cyclobutyl, phenyl, ethyl, or benzo[d][1,3]dioxolyl.

9. A compound according to claim 1 or 8, or enantiomers thereof, pharmaceutically acceptable salts thereof or mixtures thereof, wherein R² is cyclopropyl, cyclobutyl, cyclopentyl, cyclohexyl, cycloheptyl, cyclohexylmethyl, methyl, isopropyl, tetrahydropyranyl, pyridinyl, or phenyl.

10. A compound selected from:
(4-Cyclohexyl-piperazin-1-yl)-[6-(tetrahydro-pyran-4-yl)-6-aza-spiro[2.5]oct-1-yl]-methanone;
(4-Cyclohexylmethyl-piperazin-1-yl)-[6-(tetrahydro-pyran-4-yl)-6-aza-spiro[2.5]oct-1-yl]-methanone;
[6-(2-Ethyl-butyl)-6-aza-spiro[2.5]oct-1-yl]-(4-methyl-piperazin-1-yl)-methanone;
(4-Methyl-piperazin-1-yl)-[6-((E)-3-phenyl-allyl)-6-aza-spiro[2.5]oct-1-yl]-methanone;
(4-Methyl-piperazin-1-yl)-(6-phenethyl-6-aza-spiro[2.5]oct-1-yl)-methanone;
(4-Methyl-piperazin-1-yl)-[6-(1-methyl-piperidin-4-yl)-6-aza-spiro[2.5]oct-1-yl]-methanone; (4-Methyl-piperazin-1-yl)-[6-(tetrahydro-pyran-4-yl)-6-aza-spiro[2.5]oct-1-yl]-methanone; (4-Cyclobutylpiperazin-1-yl)(6-(tetrahydro-2H-pyran-4-yl)-6-azaspiro[2.5]octan-1-yl)methanone;
(4-Cyclopropylpiperazin-1-yl)(6-(tetrahydro-2H-pyran-4-yl)-6-azaspiro[2.5]octan-1-yl)methanone;
(4-Isopropylpiperazin-1-yl)(6-(tetrahydro-2H-pyran-4-yl)-6-azaspiro[2.5]octan-1-yl)methanone;
(4-Cycloheptylpiperazin-1-yl)(6-(tetrahydro-2H-pyran-4-yl)-6-azaspiro[2.5]octan-1-yl)methanone;
(4-cyclopentylpiperazin-1-yl)(6-(tetrahydro-2H-pyran-4-yl)-6-azaspiro[2.5]octan-1-yl)methanone;
4-Pyridin-2-yl-piperazin-1-yl)-[6-(tetrahydro-pyran-4-yl)-6-aza-spiro[2.5]oct-1-yl]-methanone;
(4-Pyridin-4-yl-piperazin-1-yl)-[6-(tetrahydro-pyran-4-yl)-6-aza-spiro[2.5]oct-1-yl]-methanone;
[4-(4-Methoxy-phenyl)-piperazin-1-yl]-[6-(tetrahydro-pyran-4-yl)-6-aza-spiro[2.5]oct-1-yl]-methanone;
(4-Cyclohexyl-piperazin-1-yl)-(6-phenethyl-6-aza-spiro[2.5]oct-1-yl)-methanone;
(6-Cyclohexyl-6-aza-spiro[2.5]oct-1-yl)-(4-cyclohexyl-piperazin-1-yl)-methanone;
(4-Cyclohexyl-piperazin-1-yl)-[6-(1-methyl-piperidin-4-yl)-6-aza-spiro[2.5]oct-1-yl]-methanone;
(4-Cyclohexyl-piperazin-1-yl)-(6-isopropyl-6-aza-spiro[2.5]oct-1-yl)-methanone;
(6-Cyclobutyl-6-aza-spiro[2.5]oct-1-yl)-[4-(tetrahydro-pyran-4-yl)-piperazin-1-yl]-methanone;
(6-Benzyl-6-aza-spiro[2.5]oct-1-yl)-(4-cyclohexyl-piperazin-1-yl)-methanone;
[6-(Tetrahydro-pyran-4-yl)-6-aza-spiro[2.5]oct-1-yl]-[4-(tetrahydro-pyran-4-yl)-piperazin-1-yl]-methanone;
[6-(2-Benzyloxy-ethyl)-6-aza-spiro[2.5]oct-1-yl]-(4-cyclohexyl-piperazin-1-yl)-methanone;
(4-Cyclobutyl-piperazin-1-yl)-[6-(tetrahydro-thiopyran-3-yl)-6-aza-spiro[2.5]oct-1-yl]-methanone;
(4-Cyclobutyl-piperazin-1-yl)-(6-cyclohexyl-6-aza-spiro[2.5]oct-1-yl)-methanone;
(4-Cyclobutyl-piperazin-1-yl)-[6-(tetrahydro-pyran-3-yl)-6-aza-spiro[2.5]oct-1-yl]-methanone;
(4-Cyclobutyl-piperazin-1-yl)-[6-(tetrahydro-furan-3-yl)-6-aza-spiro[2.5]oct-1-yl]-methanone;
(4-Cyclobutyl-piperazin-1-yl)-[6-(2-morpholin-4-yl-ethyl)-6-aza-spiro[2.5]oct-1-yl]-methanone;
(4-Cyclohexyl-piperazin-1-yl)-(6-pyridin-4-yl-6-aza-spiro[2.5]oct-1-yl)-methanone;
[1-(4-Cyclobutyl-piperazine-1-carbonyl)-6-aza-spiro[2.5]oct-6-yl]-acetic acid tert-butyl ester;
2-[1-(4-Cyclobutyl-piperazine-1-carbonyl)-6-aza-spiro[2.5]oct-6-yl]-N-methyl-acetamide;
(4-Cyclobutyl-piperazin-1-yl)-[6-(4-methanesulfonyl-phenyl)-6-aza-spiro[2.5]oct-1-yl]-methanone;
(4-Cyclobutyl-piperazin-1-yl)-[6-(4-methoxy-phenyl)-6-aza-spiro[2.5]oct-1-yl]-methanone;
4-[1-(4-Cyclobutyl-piperazine-1-carbonyl)-6-aza-spiro[2.5]oct-6-yl]-benzoic acid methyl ester;
(4-Cyclobutyl-piperazin-1-yl)[6-(6-methoxy-pyridin-2-yl)-6-aza-spiro[2.5]oct-1-yl]-methanone;
[6-(6-Chloro-pyridin-2-yl)-6-aza-spiro[2.5]oct-1-yl]-(4-cyclobutyl-piperazin-1-yl)-methanone;
(4-Cyclohexyl-piperazin-1-yl)-[6-(2-methoxy-ethyl)-6-aza-spiro[2.5]oct-1-yl]-methanone;
(4-Cyclobutyl-piperazin-1-yl)-(6-pyridin-2-yl-6-aza-spiro[2.5]oct-1-yl)-methanone;
(4-Cyclobutyl-piperazin-1-yl)-(6-p-tolyl-6-aza-spiro[2.5]oct-1-yl)-methanone;
4-Cyclohexyl-piperazin-1-yl)-[6-(5-trifluoromethyl-pyridin-2-yl)-6-aza-spiro[2.5]oct-1-yl]-methanone;
(6-Benzo[1,3]dioxol-5-yl-6-aza-spiro[2.5]oct-1-yl)-(4-cyclohexyl-piperazin-1-yl)-methanone;
(4-(pyridin-3-yl)piperazin-1-yl)(6-(tetrahydro-2H-pyran-4-yl)-6-azaspiro[2.5]octan-1-yl)methanone;
(4-(2-methylpyridin-4-yl)piperazin-1-yl)(6-(tetrahydro-2H-pyran-4-yl)-6-azaspiro[2.5]octan-1-yl)methanone; and
(4-(3-methylpyridin-4-yl)piperazin-1-yl)(6-(tetrahydro-2H-pyran-4-yl)-6-azaspiro[2.5]octan-1-yl)methanone; and
enantiomers thereof, pharmaceutically acceptable salts thereof, or mixtures thereof.

11-13. (canceled)

14. A pharmaceutical composition comprising at least one compound according to claim 1 or 10, or enantiomers thereof, pharmaceutically acceptable salts thereof or mixtures thereof, and a pharmaceutically acceptable carrier and/or diluent.

15. (canceled)

16. A method comprising treating at least one disorder selected from cognitive deficit in schizophrenia, narcolepsy, obesity, attention deficit hyperactivity disorder, and Alzheimer's disease in a warm blooded animal by administering to said animal in need of such treatment a therapeutically effective amount of at least one compound according to claim 1, or enantiomer thereof, pharmaceutically acceptable salt thereof, or mixture thereof.

17. A method according to claim 16, wherein said disorder is cognitive deficit in schizophrenia.

18. A method according to claim 16, wherein said disorder is Alzheimer's disease.

19. A method according to claim 16, wherein said disorder is obesity.

20. A method according to claim 16, wherein said disorder is attention deficit hyperactivity disorder.

21. A method according to claim 16, wherein said disorder is narcolepsy.

22. A method comprising treating at least one disorder selected from cognitive deficit in schizophrenia, narcolepsy, obesity, attention deficit hyperactivity disorder, and Alzheimer's disease in a warm blooded animal by administering to said animal in need of such treatment a pharmaceutical composition according to claim 14.
